(19) United States
(12) Patent Application Publication
(10) Pub. No.: US 2024/0175001 A1
(43) Pub. Date: May 30, 2024

(54) Clostridial Neurotoxins Comprising an Exogenous Activation Loop

(71) Applicant: IPSEN BIOPHARM LIMITED, Wrexham (GB)

(72) Inventors: Sai Man LIU, Wrexham (GB); Vineeta TRIPATHI, Wrexham (GB); Shilpa PALAN, Wrexham (GB); Kevin MOORE, Wrexham (GB); Karen DELAHAY, Wrexham (GB)

(21) Appl. No.: 18/552,599

(22) PCT Filed: Mar. 25, 2022
(86) PCT No.: PCT/GB2022/050756

§ 371 Date: Sep. 26, 2023

(30) Foreign Application Priority Data
Mar. 26, 2021 (GB) 2104294.0

(51) Int. Cl. C12N 9/64; A61K 8/66; A61K 38/00; A61Q 19/08; C12N 15/63

(52) U.S. Cl. CPC C12N 9/6489; A61K 8/66; A61Q 19/08; C12N 15/63; A61K 38/00; A61K 2800/86; C12Y 304/24069

(57) ABSTRACT

The present invention relates to a method for proteolytically processing a single-chain clostridial neurotoxin into a corresponding di-chain clostridial neurotoxin, the method comprising: providing a single-chain clostridial neurotoxin; and contacting the single-chain clostridial neurotoxin with furin; wherein the single-chain clostridial neurotoxin has an activation loop comprising or consisting of the polypeptide sequence Arg-Xaa-Xaa-Arg; and wherein furin hydrolyses a peptide bond of the activation loop thereby producing a di-chain clostridial neurotoxin. The invention also relates to engineered clostridial neurotoxins and methods for manufacturing the same, as well as related pharmaceutical compositions, nucleotide sequences, and therapeutic and cosmetic uses.

### FIELD OF THE INVENTION

The present invention relates to clostridial neurotoxins and methods for activating and using the same.

### BACKGROUND OF THE INVENTION

Bacteria in the genus Clostridia produce highly potent and specific protein toxins, which can poison neurons and other cells to which they are delivered. Examples of such clostridial neurotoxins include the neurotoxins produced by C. tetani (TeNT) and by C. botulinum (BoNT) serotypes A-G, and X (see WO 2018/009903 A2), as well as those produced by C. baratii and C. butyricum.

Among the clostridial neurotoxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

Clostridial neurotoxins are expressed as single-chain polypeptides in Clostridium. Each clostridial neurotoxin has a catalytic light chain separated from the heavy chain (encompassing the N-terminal translocation domain and the C-terminal receptor binding domain) by an exposed region called the activation loop. During protein maturation proteolytic cleavage of the activation loop separates the light and heavy chain of the clostridial neurotoxin, which are held together by a disulphide bridge, to create fully active di-chain toxin.

This activation process must be reproduced during standard production of recombinant toxin production. Exogenous proteases such as trypsin or Lys-C with well-defined cleavage motifs are used for proteolytically activating single-chain clostridial neurotoxins in conventional production methods. However, for some clostridial neurotoxins, incubation with Lys-C or trypsin results in partial or improper cleavage of the single-chain polypeptide resulting in the production of contaminating single-chain and/or inactive cleavage/degradation products (e.g. in the case of BoNT/E). For instance, for Botulinum neurotoxin serotype X (BoNT/X, see WO 2018/009903 A2), activation is problematic, with cleavage using trypsin or Lys-C completely degrading the polypeptide. Thus, at present there is no universal exogenous protease for activation of clostridial neurotoxins. This is particularly problematic upon identification of a new clostridial neurotoxin or production of a modified (e.g. chimeric or hybrid) neurotoxin, which requires screening of multiple proteases to determine correct activation. For re-targeted clostridial neurotoxins, some standard proteases used for activation can also cleave within the exogenous targeting moieties, resulting in incorrectly processed proteins with reduced targeting to the desired cell type. To avoid such off-target cleavage, either alternative targeting moieties must be identified (which may not always be possible), or the targeting moieties must be designed to remove the cleavage site for the standard protease, which may negatively impact the structure of the targeting moiety, and/or add to design and production costs.

Furthermore, in vitro activation of clostridial neurotoxins is associated with numerous disadvantages. There is a cost associated with the use of an exogenous protease (particularly GMP-grade protease), and its removal following activation of the clostridial neurotoxin. Dependence on a single or limited number of suppliers for GMP-grade protease can also create weakness in the supply/production chain. Purification of the activated clostridial neurotoxin from the activating exogenous protease can also affect production efficiency and yield. In addition, production of active di-chain clostridial neurotoxins according to conventional production methods necessitates strict safety and control procedures, also adding to production costs and time. Strict safety precautions are also required for practitioners working with active di-chain clostridial neurotoxins.

The present invention overcomes one or more of the above-mentioned problems.

### SUMMARY OF THE INVENTION

The protease furin is expressed in vivo in a wide range of tissues, including the brain, endocrine tissue, the lungs, liver, gastrointestinal tract, liver, kidneys and bladder, and by a wide range of cell types. Furin expression is mainly localised to the Golgi apparatus and the nucleoplasm of cells. The furin protease recognises and cleaves immediately C-terminal to an Arg-Xaa-Yaa-Arg (where Xaa and Yaa are any amino acid) peptide sequence (SEQ ID NO: 1), particularly Arg-Xaa-Lys-Arg (SEQ ID NO: 2), Arg-Xaa-Arg-Arg (SEQ ID NO: 3) or Arg-Lys-Lys-Arg (SEQ ID NO: 4). Notably, such furin cleavage sites are absent from all of the clostridial neurotoxin activation loops (see FIG. 1). Thus, furin has previously been ruled out as a protease for use in activating clostridial neurotoxins.

The present inventors are the first to demonstrate that insertion of a furin cleavage site into a clostridial neurotoxin allows for the in vivo activation of clostridial neurotoxins. This is a paradigm shift in terms of clostridial neurotoxin production, processing and activation, and indeed therapeutic use. In particular, to the extent that attempts have previously been made in the art to introduce exogenous cleavage sites into clostridial neurotoxins, the goal has always been to facilitate in vitro production and processing of clostridial neurotoxins which are then administered in di-chain form. The present inventors are the first to appreciate the potential of in vivo activation of clostridial neurotoxins, to demonstrate that in vivo activation of clostridial neurotoxins is possible, and that this can be achieved by the insertion of a furin cleavage site. This was particularly surprising given that the consensus in the art (e.g. WO 2020/065336, which is herein incorporated by reference), is that insertion of exogenous cleavage sites, even in the context of in vitro activation, can give rise to conformational changes which can have a negative effect on cleavage efficiency, such that complete replacement of the activation loop, rather than insertion of an exogenous activation site, is conventionally preferred.

Furthermore, not only have the present inventors shown that insertion of a furin cleavage site is possible and that this can be used to activate clostridial neurotoxins in vivo, but they have also surprisingly shown that furin activation of clostridial neurotoxins can achieve comparable potency to conventionally activated clostridial neurotoxin.

In addition, the furin-activated engineered clostridial neurotoxins of the invention offer several potential benefits compared with conventionally activated clostridial neurotoxins, such as improving the safety of operators (e.g. clinicians or others handling the furin-activated engineered neurotoxins of the invention in order to administer to patients, and workers involved in the production of the furin-activated engineered neurotoxins), and/or reducing manufacturing burden/costs. The furin-activated engineered neurotoxins of the invention also have potentially increased safety profiles for patients. In particular, the present inventors have demonstrated that partial replacement of the endogenous (native) activation loop of botulinum neurotoxin serotype A (BoNT/A) with a furin cleavage site produced an engineered BoNT/A that is activated from the single-chain to the di-chain form in vivo in mice, and that has comparable potency in mouse models with native BoNT/A (produced in Clostridium bacteria and activated by a native protease within the bacteria) and recombinant BoNT/A that had been activated in vitro prior to administration. In addition, the inventors demonstrated that an engineered BoNT/A with a furin cleavage site potentially had a faster onset of action in mice compared with the recombinant BoNT/A active di-chain (as illustrated in the Examples herein). Equivalent potency combined with other exemplified properties is suggestive of a potentially improved safety profile and therapeutic window for furin-activated engineered BoNT/A. In addition, the inventors have also shown that single-chain BoNT/A1 it capable of eliciting a therapeutic effect (as evidence by the DAS score elicited using single-chain BoNT/A1 in the Examples herein). Thus, the inventors have demonstrated for the first time that single-chain clostridial neurotoxins, such as engineered BoNT/A1 with a furin cleavage site and single-chain BoNT/A1, have therapeutic potential, without requiring activation to di-chain form prior to administration.

Accordingly, the invention provides an engineered clostridial neurotoxin, comprising a furin cleavage site, wherein cleavage at said furin cleavage site results in the production of a di-chain form of the engineered clostridial neurotoxin.

The furin cleavage site may comprise an amino acid sequence Arg-Xaa-Xaa-Arg (SEQ ID NO: 1), preferably Arg-Xaa-Lys/Arg-Arg (SEQ ID NOs: 2 and 3), even more preferably Arg-Lys-Lys-Arg (SEQ ID No: 4), and even more preferably KQKSSNSRKKR (SEQ ID NO: 5). The engineered clostridial neurotoxin may comprise an exogenous activation loop which comprises or consists of any one of SEQ ID NOs: 14 to 22), preferably SEQ ID NO: 22. An endogenous activation loop of a clostridial neurotoxin or part thereof may be replaced by a furin cleavage site. The endogenous neurotoxin activation loop may be one or more selected from SEQ ID NO: 34 to 57.

The clostridial neurotoxin may be selected from: (a) a Botulinum Neurotoxin (BoNT) serotype A, serotype B, serotype C, serotype D, serotype E, serotype F, serotype G or serotype X, or a Tetanus Neurotoxin (TeNT); or (b) a chimeric BONT or a hybrid BONT. The engineered clostridial neurotoxin may be BoNT/A, optionally BoNT/A1. The engineered clostridial neurotoxin may be a single-chain clostridial neurotoxin: (a) encoded by a nucleotide sequence having at least 70% sequence identity to SEQ ID NO: 23; and/or (b) comprising a polypeptide sequence having at least 70% sequence identity to one or more of SEQ ID NOs: 24 or SEQ ID NO: 70 to 78.

The engineered clostridial neurotoxin may be a re-targeted clostridial neurotoxin in which an endogenous HC or HCC of a clostridial neurotoxin is replaced by an exogenous targeting moiety (TM).

The invention also provides an engineered BoNT/A comprising a furin cleavage site, which comprises a polypeptide sequence having at least 70% sequence identity, preferably at least 80%, more preferably at least 90%, even more preferably at least 95% sequence identity to one or more of SEQ ID NOs: 24.

The invention further provides a method for proteolytically processing an engineered clostridial neurotoxin (e.g. an engineered BoNT/A) of the invention into a corresponding di-chain clostridial neurotoxin (e.g. engineered BoNT/A), the method comprising contacting the engineered clostridial neurotoxin (e.g. engineered BoNT/A) with furin, thereby producing a di-chain clostridial neurotoxin (e.g. BoNT/A). The invention further provides a di-chain clostridial neurotoxin or BoNT/A obtainable by said method.

The invention also provides a polynucleotide encoding an engineered clostridial neurotoxin or an engineered BoNT/A of the invention. The invention further provides an expression vector comprising a polynucleotide of the invention, which is operably linked to a promoter. Said polynucleotide or expression vector may: (a) comprise a nucleotide sequence having at least 70% sequence identity to SEQ ID NO: 23; and/or (b) encode a polypeptide sequence having at least 70% sequence identity to one or more of SEQ ID NOs: 24 or 70 to 78.

The invention further provides a method of producing an engineered clostridial neurotoxin or an engineered BoNT/A of the invention, said method comprising the step of expressing a polynucleotide or an expression vector of the invention in a cell, and recovering the expressed engineered clostridial neurotoxin or engineered BoNT/A. Said method may further comprise a step of introducing the polynucleotide or expression vector into the cell.

The invention also provides a cell expressing an engineered clostridial neurotoxin or an engineered BoNT/A of the invention. Said cell may comprise a polynucleotide or an expression vector of the invention.

The invention also provides a pharmaceutical composition comprising an engineered clostridial neurotoxin, an engineered BoNT/A, a di-chain clostridial neurotoxin or di-chain BONT/A of the invention, and a pharmaceutically acceptable carrier, excipient, diluent, adjuvant, propellant and/or salt.

The invention further provides an engineered clostridial neurotoxin, an engineered BONT/A, a di-chain clostridial neurotoxin or di-chain BoNT/A of the invention, or a pharmaceutical composition of the invention, for use in a method of preventing or treating a disease or disorder for which a therapy with a botulinum neurotoxin is indicated, wherein optionally said disease or disorder is selected from a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder.

The invention further provides the use of an engineered clostridial neurotoxin, an engineered BoNT/A, a di-chain clostridial neurotoxin or di-chain BoNT/A of the invention, or a pharmaceutical composition of the invention, in the manufacture of a medicament for preventing or treating a disease or disorder for which a therapy with a botulinum neurotoxin is indicated, wherein optionally said disease or disorder is selected from a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder.

The invention further provides a cosmetic composition comprising an engineered clostridial neurotoxin, an engineered BoNT/A, a di-chain clostridial neurotoxin or di-chain BoNT/A of the invention, and a cosmetically acceptable carrier, excipient, diluent, adjuvant, propellant and/or salt.

The invention also provides the use of a cosmetic composition of the invention, for preventing or alleviating a cosmetic indication for which the application of a botulinum neurotoxin is indicated.

The invention further provides a method for proteolytically processing a single-chain clostridial neurotoxin into a corresponding di-chain clostridial neurotoxin, the method comprising: (a) providing a single-chain clostridial neurotoxin; and (b) contacting the single-chain clostridial neurotoxin with furin; wherein the single-chain clostridial neurotoxin has an activation loop comprising or consisting of the polypeptide sequence Arg-Xaa-Xaa-Arg (SEQ ID NO: 1); and wherein furin hydrolyses a peptide bond of the activation loop thereby producing a di-chain clostridial neurotoxin. The activation loop may comprise or consist of: (a) Arg-Xaa-Lys/Arg-Arg (SEQ ID NOs: 2 or 3); (b) Arg-Lys-Lys-Arg (SEQ ID No: 4); and/or (c) KQKSSNSRKKR (SEQ ID NO: 5). The single-chain clostridial neurotoxin may: (a) be an engineered clostridial neurotoxin of the invention; (b) be encoded by a nucleotide sequence having at least 70% sequence identity to SEQ ID NO: 23; and/or (c) comprise a polypeptide sequence having at least 70% sequence identity to one or more of SEQ ID NOs: 24 or 70 to 78.

The invention also provides a clostridial neurotoxin, or a pharmaceutical composition comprising said clostridial neurotoxin, for use in a method of preventing or treating a disease or disorder for which a therapy with a botulinum neurotoxin is indicated, wherein the clostridial neurotoxin is administered to a subject in single-chain form. Said clostridial neurotoxin or pharmaceutical composition may be substantially free of a di-chain form of the clostridial neurotoxin. The clostridial neurotoxin, or a pharmaceutical composition may comprise less than 400 pg di-chain clostridial neurotoxin per 100 ng single-chain clostridial neurotoxin, or less than 300 pg di-chain clostridial neurotoxin per 100 ng single-chain clostridial neurotoxin, or less than 200 pg di-chain clostridial neurotoxin per 100 ng single-chain clostridial neurotoxin, or less than 100 pg di-chain clostridial neurotoxin per 100 ng single-chain clostridial neurotoxin, or less than 50 pg di-chain clostridial neurotoxin per 100 ng single-chain clostridial neurotoxin. Said disease or disorder may be selected from a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder.

The invention further provides the use of a cosmetic composition comprising a single-chain clostridial neurotoxin, and a cosmetically acceptable carrier, excipient, diluent, adjuvant, propellant and/or salt for preventing or alleviating a cosmetic indication for which the application of a botulinum neurotoxin is indicated, wherein the single-chain clostridial neurotoxin is administered to a subject in single-chain form.

### BRIEF DESCRIPTION OF THE DRAWINGS

FIG. 1: shows a comparison of the protein sequence of the activation loop for all BONT serotypes and a tetanus toxin with two flanking cysteines forming a disulphide bridge connecting the light and heavy chain of a toxin molecule. Factor Xa cleavage site (IDGR) in BONT/C1 and BoNT/CD underlined.

FIG. 2: Protein gel and Western blot confirming successful cleavage of BoNT/A1-furin protein SEQ ID NO: 24 (SXN104539) by furin.

FIG. 3: Percentage SNAP-25 cleavage in rat embryonic spinal cord neurons (eSCN) for native BoNT/A1 (squares), wild-type single chain rBoNT/A1 (diamonds), and single chain rBoNT/A1 with the furin loop (circles). Rat embryonic spinal cord neurons were cultured for three weeks and treated separately with each BONT molecule for 24 h, before Western blotting with SNAP-25 specific antibody. Data is mean +SEM from three independent experiments in triplicate.

FIG. 4: The potency (t50) of wild-type dichain rBoNT/A1 (triangles), wild-type single chain rBoNT/A1 (plus), and single chain rBoNT/A1 with the furin loop (circles) in the mouse phrenic nerve hemi-diaphragm assay (mPNHD). (A) Mouse phrenic nerve hemi-diaphragm tissue was incubated with the BoNT molecules as indicated. Diaphragm contractile force was recorded until the contraction was no longer detectable or after 140 minutes. Each point corresponds to independent determinations. (B) The ts0 value is the time required to inhibit the contractile force of the mouse hemi-diaphragm by 50%.

FIG. 5: The effect of wild-type dichain rBoNT/A1 (squares), wild-type single chain rBoNT/A1 (diamonds), and single chain rBoNT/A1 with the furin loop (circles) on body weight following injection into the peronei or gastrocnemius lateralis. (A) The curves correspond to mean body weights observed 1, 2, 3 or 4 days post-administration. (B) The curves correspond to mean body weights observed up to 25 days post-administration. All values are means±standard error of the mean.

FIG. 6: The efficacy of wild-type dichain rBoNT/A1 (squares), wild-type single chain rBoNT/A1 (diamonds), and single chain rBoNT/A1 with the furin loop (circles) in the mean peak digit abduction score (DAS) following injection into the peronel or gastrocnemius lateralis. The curves correspond to mean peak DAS responses observed 1, 2, 3 or 4 days post-administration. All values are means±standard error of the mean.

FIG. 7: The duration of wild-type dichain rBoNT/A1 (squares), wild-type single chain rBoNT/A1 (diamonds), and single chain rBoNT/A1 with the furin loop (circles) on body weight following injection into the peronei or gastrocnemius lateralis. The curves correspond to mean peak DAS responses observed up to 600 hours post-administration. All values are means±standard error of the mean.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition.

It should be understood that this invention is not limited to the particular methodology, protocols, and reagents, etc., described herein and as such can vary. In particular, any methods and materials similar or equivalent to those described herein can be used in the practice or testing of embodiments of this disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

A “fragment” of a polypeptide typically comprises at least 50%, at least 60%, at least 70%, at least 80%, at least 90%, at least 95%, at least 97% or more of the original polypeptide.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” encompasses a complete inhibition or reduction as compared to a reference level. “Complete inhibition” is a 100% inhibition (i.e. abrogation) as compared to a reference level.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

An individual can be one who has been previously diagnosed with or identified as suffering from or having a condition in need of treatment or one or more complications related to such a condition, and optionally, have already undergone treatment for a condition as defined herein or the one or more complications related to said condition. Alternatively, an individual can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

An “individual in need” of treatment for a particular condition can be an individual having that condition, diagnosed as having that condition, or at risk of developing that condition.

The terms “subject”, “individual” and “patient” are used interchangeably herein to refer to a mammalian individual. An “individual” may be any mammal. Generally, the individual may be human; in other words, in one embodiment, the “individual” is a human. A “individual” may be an adult, juvenile or infant. An “individual” may be male or female.

The term “pharmaceutically acceptable” as used herein means approved by a regulatory agency of the Federal or a state government, or listed in the U.S. Pharmacopeia, European Pharmacopeia or other generally recognized pharmacopeia.

The publications discussed herein are provided solely for their disclosure prior to the filing date of the present application. Nothing herein is to be construed as an admission that such publications constitute prior art to the claims appended hereto.

### Engineered Clostridial Neurotoxins

The present invention provides an engineered clostridial neurotoxin, comprising a furin cleavage site. Typically cleavage at said furin cleavage site results in the production of a di-chain form of the engineered clostridial neurotoxin. In other words, cleavage at the furin cleavage site results in activation of an engineered clostridial neurotoxin. The endogenous (native) activation loop of a clostridial neurotoxin may be replaced (or partially replaced) by a furin cleavage site. As such the term “furin cleavage site” may be used interchangeably with the terms “furin activation site”, and an exogenous activation loop as defined herein will typically comprise or consist of a furin cleavage site. The engineered clostridial neurotoxins of the invention may be activated in vivo. Thus, the engineered clostridial neurotoxins open up a new field of processing and therapeutic use for clostridial neurotoxins, enabling toxins to be produced and administered as single-chain clostridial neurotoxins, which are then cleaved to produce the active di-chain form in vivo. As described herein, this represents a paradigm shift in the field of clostridial neurotoxins.

The clostridial neurotoxin (pre-engineering) is typically characterised in that the endogenous activation loop is inefficiently proteolytically processed by furin. In contrast to the clostridial neurotoxin (pre-engineering), an engineered clostridial neurotoxin of the invention is not inefficiently proteolytically processed by furin and/or a peptide bond outside of the exogenous activation loop of the engineered clostridial neurotoxin is not hydrolysed by furin. Thus, the clostridial neurotoxin (pre-engineering) is typically resistant to proteolytic processing by furin. The terms “inefficiently proteolytically processed by furin”, “resistant to proteolytic processing by furin”, “not substantially hydrolysed by furin” “inefficiently activated by furin”, “resistant to activation by furin” and “not substantially activated by furin” are used interchangeably herein.

A clostridial neurotoxin (pre-engineering) is typically one in which a peptide bond (either within or outside of the activation loop) is not, or is not substantially, hydrolysed by furin. The term “not substantially hydrolysed” means that less than 10%, 5%, 4%, 3%, 2% or 1% of the clostridial neurotoxin present in a reaction contains a peptide bond that has been hydrolysed by furin in a method of the invention.

Accordingly, a clostridial neurotoxin (pre-engineering) typically does not contain a furin cleavage site (e.g. as defined herein) within its endogenous activation loop. Therefore, in some embodiments, the invention relates to clostridial neurotoxins (pre-engineering) that are not the BoNT/DC of UniProt Accession No. AB745660 (version 1 of the sequence, accessed 19 Jan. 2022), BoNT/C1 of UniProt Accession No. P18640 (version 3 of the sequence, accessed 23 Mar. 2022), BoNT/CD of UniProt Accession No. Q5DW55 (version 1 of the sequence, accessed 23 Mar. 2022), BoNT/D of NCBI Accession No. AB012112 (version 1 of the sequence, accessed 23 Mar. 2022) and/or BoNT/F5 of UniProt Accession No. D2KHQ9 (version 1 of the sequence, accessed 23 Mar. 2022).

The invention may comprise replacing an endogenous activation loop (or part thereof) of any clostridial neurotoxin with an (exogenous) furin cleavage site or an exogenous activation loop comprising a furin cleavage site as described herein. The clostridial neurotoxin may be a botulinum neurotoxin (BoNT) or a tetanus neurotoxin (TeNT). Preferably the clostridial neurotoxin is a botulinum neurotoxin, such as BoNT/A, BoNT/B, BONT/C1, BONT/D, BONT/E, BONT/F, BONT/G or BoNT/X, or a chimeric or hybrid thereof.

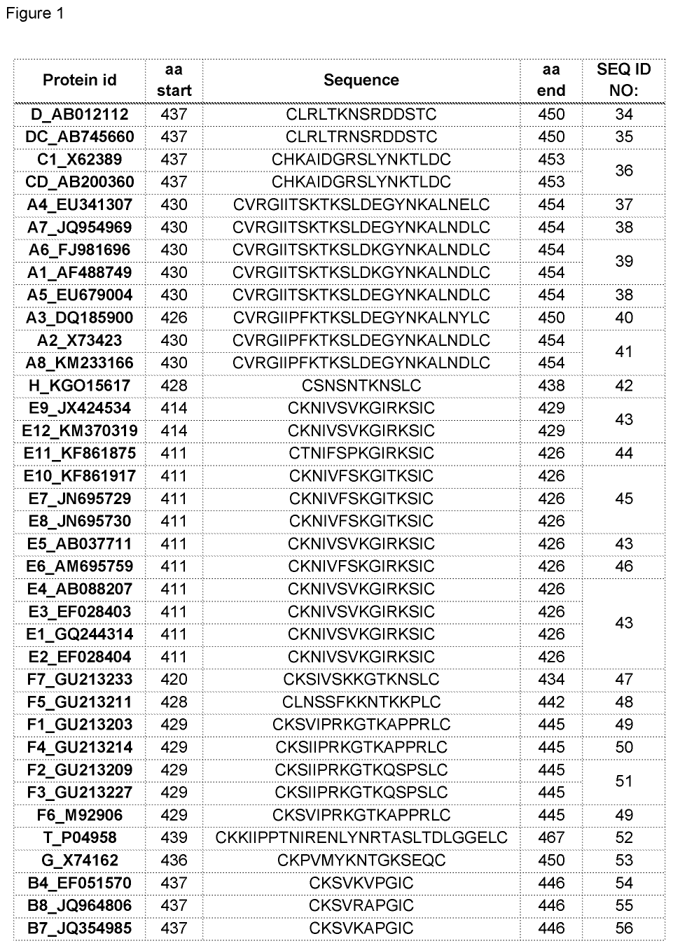

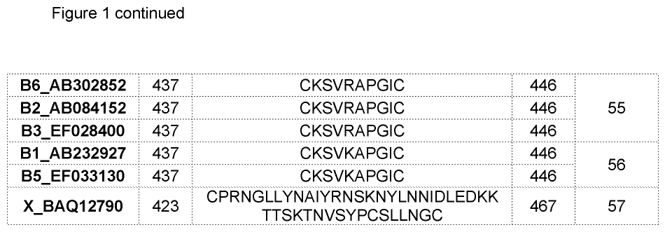

The term “endogenous activation loop” as used herein means an activation loop present in a subject clostridial neurotoxin, e.g. a subject clostridial neurotoxin of the indicated serotype. For example, BoNT/A1 includes a BoNT/A1 heavy chain and light chain, thus the endogenous activation loop of BoNT/A1 is an A1 activation loop. For clostridial neurotoxin chimeras or hybrids, the person skilled in the art can identify the “endogenous activation loop”, for example by determining the serotype(s) from which the L-chain and H_{N} domain are derived. In some embodiments, a chimera or hybrid clostridial neurotoxin may have an endogenous activation loop that is a fusion of an activation loop from two different serotypes. By way of example, a chimeric clostridial neurotoxin such as BoNT/A1C1 has a BoNT/A1 light chain and translocation domain, thus the endogenous BoNT/A1C1 activation loop is an A1 activation loop. Examples of activation loops are provided in FIG. 1. The endogenous activation loop, is typically bounded by cysteine residues that form a disulphide bridge and covalently link the light and heavy chains of a (pre-engineering) clostridial neurotoxin. Thus, an endogenous activation loop sequence may be recited including the bounding cysteine residues (as described herein), or without the bounding cysteine residues. One of ordinary skill in the art would understand that the definitions may be used interchangeably, and would readily be able to identify an endogenous activation loop, either including or excluding the bounding cysteine residues.

Typically an “endogenous activation loop” is any activation loop that is does not comprise or consist of SEQ ID NO: 1. Preferably “endogenous activation loop” is any activation loop that is not SEQ ID NO: 2, SEQ ID NO: 3, SEQ ID NO: 4 and/or SEQ ID NO: 5.

By contrast, an “exogenous activation loop” as used herein means an activation loop that is different to the endogenous activation loop present in a subject clostridial neurotoxin, e.g., a subject clostridial neurotoxin of the indicated serotype, and wherein the exogenous activation loop comprises a furin cleavage site. For example, a BoNT/C1 activation loop has a different polypeptide sequence to a wild-type BoNT/A1 activation loop, therefore the BoNT/C1 activation loop is exogenous to BoNT/A1. For clostridial neurotoxin chimeras or hybrids, the person skilled in the art can determine whether an activation loop is an “exogenous activation loop”, for example by determining the serotype(s) from which the L-chain and H_{N} domain are derived. For example, where the L-chain is a BoNT/B L-chain and the H_{N} domain is from BoNT/D, the endogenous activation loop may have a portion of a BoNT/B sequence and a portion of a BoNT/D sequence, and if an activation loop (e.g. a C1 activation loop) is different thereto, and comprises a furin cleavage site, it is considered an “exogenous activation loop”.

Determination of whether an activation loop is an “endogenous activation loop” may be made by aligning the sequence of a subject clostridial neurotoxin with the activation loop, and seeing if the activation loop is present in the subject clostridial neurotoxin sequence. If it is present, then the activation loop can be identified as an endogenous activation loop. As described herein, the endogenous activation loop of a clostridial neurotoxin is replaced by an exogenous cleavage site which is a furin cleavage site, or by an exogenous activation loop which comprises a furin cleavage site.

Typically according to the invention a furin cleavage site is inserted between the two cysteine residues that bound the endogenous activation loop of a pre-engineering clostridial neurotoxin, although the precise position of the furin cleavage site within the endogenous activation loop is not limited, provided that the conformation of the resultant engineered clostridial neurotoxin is not disrupted and/or the engineered clostridial neurotoxin rendered non-functional.

The entire endogenous activation loop replaced by a furin cleavage site or an exogenous activation loop comprising a furin cleavage site as described herein. Alternatively, a part or portion of the endogenous activation loop may be replaced (also referred to herein as partial replacement of the endogenous activation loop), such as at least 5, 10, 15, 20, 25, 30, 35 or 40 amino acid residues of the endogenous activation loop are replaced. Preferably 5 to 20, more preferably 5 to 15 amino acid residues of the endogenous activation loop are replaced. Typically partial replacement involves the replacement of consecutive amino acids within the endogenous activation loop.

Replacement of an endogenous activation loop may be achieved by any method known in the art. For example, replacement might be achieved by way of an amino acid modification. An endogenous activation loop may be replaced by deleting one or more amino acid residues of the endogenous activation loop. An endogenous activation loop may be replaced by substituting one or more amino acid residues of the endogenous activation loop with amino acid residues of an exogenous activation loop. An endogenous activation loop (or a portion thereof) may be deleted, and a furin cleavage site or an exogenous activation loop comprising a furin cleavage site inserted, preferably at the position formally occupied by the endogenous activation loop. Alternatively, the endogenous activation loop may be retained in an engineered clostridial neurotoxin of the invention, and preferably inactivated (e.g. by way of mutation). It is preferred that the endogenous activation loop (a portion thereof or the entire endogenous activation loop) is not present in the engineered clostridial neurotoxin of the invention. It is preferred that the furin cleavage site or the exogenous activation loop comprising the furin cleavage site occupies the position in the clostridial neurotoxin formally occupied by the endogenous activation loop. For the avoidance of doubt, when an endogenous activation loop is modified to comprise a furin cleavage site (e.g. by substitution of residues within the endogenous activation loop or by the addition of one or more amino acids to form a furin cleavage site within the endogenous activation loop), the modified activation loop is an exogenous activation loop according to the invention. Therefore, potentially an engineered clostridial neurotoxin can comprise both its endogenous activation/cleavage site and a furin cleavage site, and as such may be activated either by the native activating protease (or equivalents used in recombinant BONT production, e.g. trypsin or Lys-C), or by furin.

Methods for modifying proteins by substitution, insertion or deletion of amino acid residues are known in the art and may be employed in the practice of the present invention. By way of example, amino acid modifications may be introduced by modification of a DNA sequence encoding a clostridial neurotoxin. This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively a modified gene sequence can be chemically synthesised.

An endogenous activation loop replaced according to the invention may comprise or consist of a polypeptide sequence having at least 70% (e.g. at least 80% or 90%) sequence identity to SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56 or SEQ ID NO: 57. In particular, an endogenous activation loop may comprise or consist of a polypeptide sequence having at least 95% sequence identity to SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56 or SEQ ID NO: 57. Preferably, an endogenous activation loop comprises or consists of a polypeptide sequence shown as SEQ ID NO: 34, SEQ ID NO: 35, SEQ ID NO: 36, SEQ

ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56 or SEQ ID NO: 57.

An endogenous activation loop replaced according to the invention may comprise or consist of a polypeptide sequence having at least 70% (e.g. at least 80% or 90%) sequence identity to SEQ ID NO: 37, SEQ ID NO: 38, SEQ ID NO: 39, SEQ ID NO: 40 or SEQ ID NO: 41. An endogenous activation loop may comprise or consist of a polypeptide sequence having at least 95% sequence identity to SEQ ID NO: 37, SEQ ID NO: 38, SEQ ID NO: 39, SEQ ID NO: 40 or SEQ ID NO: 41. Preferably, an endogenous activation loop comprises or consists of a polypeptide sequence shown as SEQ ID NO: 37, SEQ ID NO: 38, SEQ ID NO: 39, SEQ ID NO: 40 or SEQ ID NO: 41.

Preferably an endogenous activation loop replaced according to the invention comprises or consists of a polypeptide sequence having at least 70% (e.g. at least 80% or 90%) sequence identity to SEQ ID NO: 39. An endogenous activation loop may comprise or consist of a polypeptide sequence having at least 95% sequence identity to SEQ ID NO: 39. More preferably, an endogenous activation loop comprises or consists of a polypeptide sequence shown as SEQ ID NO: 39.

The present invention encompasses methods and clostridial neurotoxins in which an endogenous activation loop has been replaced by an exogenous cleavage site, which is a furin cleavage site, or an exogenous activation loop which comprises a furin cleavage site. Typically a furin cleavage site comprises or consists of the amino acid sequence Arg-Xaa-Yaa-Arg (SEQ ID NO: 1), where Xaa and Yaa may each be independently selected from any amino acid.

It is not intended that either Xaa or Yaa be limited to only one type of amino acid. Thus, one or more residues present at Xaa and Yaa may be independently selected from the standard amino acids: aspartic acid, glutamic acid, arginine, lysine, histidine, asparagine, glutamine, serine, threonine, tyrosine, methionine, tryptophan, cysteine, alanine, glycine, valine, leucine, isoleucine, proline, and phenylalanine. One or more residues present at position Yaa may be independently selected from the standard amino acids: aspartic acid, glutamic acid, arginine, lysine, histidine, asparagine, glutamine, serine, threonine, tyrosine, methionine, tryptophan, cysteine, alanine, glycine, valine, leucine, isoleucine, proline, and phenylalanine.

Alternatively/additionally, one or more residues present at Xaa and Yaa may be independently selected from a non-standard amino acid (an amino acid that is not part of the standard set of 20 described above). By way of example, non-standard amino acids may include 4-hydroxyproline, 6-N-methyl lysine, 2-aminoisobutyric acid, isovaline, α-methyl serine, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, L-Ornithine, L-2-amino-3-guanidinopropionic acid, or D-isomers of Lysine, Arginine and/or Ornithine, and 4-fluorophenylalanine. Methods for introducing non-standard amino acids into proteins are known in the art, and include recombinant protein synthesis using E. coli auxotrophic expression hosts.

Properties of the standard amino acids are indicated in the table below:

| AMINO ACID | | | SIDE CHAIN |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

Typically Xaa is a small and/or hydrophilic amino acid and/or Yaa is a positively charged amino acid. Preferred examples of furin cleavage sites of the invention include amino acid sequences comprising or consisting of Arg-Xaa-Lys-Arg (SEQ ID NO: 2), Arg-Xaa-Arg-Arg (SEQ ID NO: 3) and Arg-Lys-Lys-Arg (SEQ ID NO: 4).

A furin cleavage site of the invention may comprise or consist of a core or minimal furin cleavage site such as any one of SEQ ID NO:1, SEQ ID NO: 2, SEQ ID NO: 3 or SEQ ID NO: 4 and one or more additional amino acids. Preferably, a furin cleavage site of the invention may comprise or consist of a core or minimal furin cleavage site such as any one of SEQ ID NO: 2, SEQ ID NO: 3 or SEQ ID NO: 4 and one or more additional amino acids. The one or more additional amino acids may be either N-terminal and/or C-terminal to the core furin cleavage site. The one or more additional amino acid may be preferably selected from small and/or hydrophilic amino acids (such as serine and/or lysine).

One or more amino acid residue (such as two, three, four, five, six, seven, eight, nine or ten amino acids) immediately N-terminal to the N-terminal Arg of the core furin cleavage site (e.g. SEQ ID NO:1, SEQ ID NO: 2, SEQ ID NO: 3 or SEQ ID NO: 4) may be polar (hydrophilic) amino acid (e.g. serine or threonine), or a positively charged amino acid (e.g. lysine or arginine).

Alternatively or in addition, one or more amino acid residue (such as two, three, four, five, six, seven, eight, nine or ten amino acids) immediately C-terminal to the C-terminal Arg of the core furin cleavage site (e.g. SEQ ID NO:1, SEQ ID NO: 2, SEQ ID NO: 3 or SEQ ID NO: 4) may be polar (hydrophilic) amino acid (e.g. serine or threonine), or a positively charged amino acid (e.g. lysine or arginine).

Preferably, one or more amino acid residue (such as two, three, four, five, six, seven, eight, nine or ten amino acids) immediately N-terminal to the N-terminal Arg of the core furin cleavage site (e.g. SEQ ID NO:1, SEQ ID NO: 2, SEQ ID NO: 3 or SEQ ID NO: 4) is polar (hydrophilic) amino acid (e.g. serine or threonine), or a positively charged amino acid (e.g. lysine or arginine) and one or more amino acid residue (such as two, three, four, five, six, seven, eight, nine or ten amino acids) immediately C-terminal to the C-terminal Arg of the core furin cleavage site (e.g. SEQ ID NO:1, SEQ ID NO: 2, SEQ ID NO: 3 or SEQ ID NO: 4) may be polar (hydrophilic) amino acid (e.g. serine or threonine), or a positively charged amino acid (e.g. lysine or arginine).

Non-limiting examples of furin cleavage sites comprising one or more additional amino acids include: KQKSSNSRKKR (SEQ ID NO: 5), SRKKRS (SEQ ID NO: 6), SRKRRS (SEQ ID NO: 7), SKRKKRS (SEQ ID NO: 8), SKRKRRS (SEQ ID NO: 9), TSSKSRRRKKRSTS (SEQ ID NO: 10), AGLITGGRRTRRSI (SEQ ID NO: 11), KVADSLSTRKQKR (SEQ ID NO: 12) and LATGLRNTSQRSRRRKKRGL (SEQ ID NO: 13).

In some embodiments a furin cleavage site of the invention has at least 70% sequence identity to SEQ ID NO: 5. A furin cleavage site may have at least 80%, 85% or 90% sequence identity to SEQ ID NO: 5. Preferably a furin cleavage site has at least 95% sequence identity to SEQ ID NO: 5. More preferably, a furin cleavage site has at least 99% sequence identity to SEQ ID NO: 5. Particularly preferred is a furin cleavage site comprising or consisting of SEQ ID NO: 5.

The engineered clostridial neurotoxins of the invention may comprise an exogenous activation loop comprising any furin cleavage site as described herein. An exogenous activation loop may be produced by replacing one or more amino acids of an endogenous activation loop of a clostridial neurotoxin, as described herein. In some preferred embodiments, the replaced amino acids of the endogenous activation loop are replaced by a furin cleavage site or exogenous activation loop having the same number of amino acids. In other words, by way of illustration, if five amino acids are replaced in an endogenous activation loop, the replacement furin cleavage site or exogenous activation loop comprising said furin cleavage site has five amino acids. If ten amino acids are replaced in an endogenous activation loop, the replacement furin cleavage site or exogenous activation loop comprising said furin cleavage site has ten amino acids.

Non-limiting examples of such exogenous activation loops include CVRGIITSKTKSLSRKKRSALNDLC (SEQ ID NO: 14), CVRGIITSKTKSLSRKRRSALNDLC (SEQ ID NO: 15), CVRGIITSKTKSSKRKKRSALNDLC (SEQ ID NO: 16) CVRGIITSKTKSSKRKRRSALNDLC (SEQ ID NO: 17), CVRGITSSKSRRRKKRSTSALNDLC (SEQ ID NO: 18), CVRGIAGLITGGRRTRRSIALNDLC (SEQ ID NO: 19), CVRGIIKVADSLSTRKQKRALNDLC (SEQ ID NO: 20), CVRGILATGLRNTSQRSRRRKKRGLALNDLC (SEQ ID NO: 21) and CVRGIKQKSSNSRKKRSTSALNDLC (SEQ ID NO: 22), all of which are derived from the BONT/A1 activation loop. SEQ ID NO: 22 is a preferred example of an exogenous activation loop.

The invention provides a method for manufacturing an engineered clostridial neurotoxin according to the invention, comprising replacing an endogenous activation loop (or part thereof) of a clostridial neurotoxin by an exogenous activation loop or an exogenous cleavage site, thereby providing an engineered clostridial neurotoxin, wherein the exogenous cleavage site is a furin cleavage site as described herein, or the exogenous activation loop comprises said furin cleavage site. Typically said furin cleavage site comprises or consists of the amino acid sequence of SEQ ID NO: 1, SEQ ID NO: 2, SEQ ID NO: 3, SEQ ID NO: 4 or SEQ ID NO: 5, or the exogenous activation loop comprises said furin cleavage site.

The invention provides an engineered clostridial neurotoxin (e.g. obtainable by a method of the invention), wherein an endogenous activation loop (or part thereof) of a clostridial neurotoxin has been replaced by an exogenous activation loop or an exogenous cleavage site, thereby providing an engineered clostridial neurotoxin, wherein the exogenous cleavage site is a furin cleavage site as described herein, or the exogenous activation loop comprises said furin cleavage site. Typically said furin cleavage site comprises or consists of the amino acid sequence of SEQ ID NO: 1, SEQ ID NO: 2, SEQ ID NO: 3, SEQ ID NO: 4 or SEQ ID NO: 5, or the exogenous activation loop comprises said furin cleavage site.

A clostridial neurotoxin of the present invention (e.g. engineered clostridial neurotoxin) may be encoded by a nucleotide sequence having at least 70% sequence identity to SEQ ID NO: 23. A clostridial neurotoxin of the present invention may be encoded by a nucleotide sequence having at least 80% or 90% sequence identity to SEQ ID NO: 23. Preferably, a clostridial neurotoxin of the present invention may be encoded by a nucleotide sequence comprising (more preferably consisting of) SEQ ID NO: 23.

A clostridial neurotoxin of the present invention (e.g. engineered clostridial neurotoxin) may comprise a polypeptide sequence having at least 70% sequence identity to one or more of SEQ ID NOs: 24 or 70 to 78. A clostridial neurotoxin of the present invention may comprise a polypeptide sequence having at least 80% or 90% sequence identity to one or more of SEQ ID NOs: 24 or 70 to 78. Preferably, a clostridial neurotoxin of the present invention may comprise (more preferably consist of) a polypeptide sequence shown as any one of SEQ ID NOs: 24 or 70 to 78.

The clostridial neurotoxin of the present invention (e.g. engineered clostridial neurotoxin) is preferably BoNT/A, even more preferably BoNT/A1, wherein the clostridial neurotoxin is encoded by a nucleotide sequence having at least 70% sequence identity to SEQ ID NO: 23. The clostridial neurotoxin may be encoded by a nucleotide sequence having at least 80% or 90% sequence identity to SEQ ID NO: 23. Preferably the clostridial neurotoxin is encoded by a nucleotide sequence comprising (or consisting of) SEQ ID NO: 23. The clostridial neurotoxin of the present invention is preferably BoNT/A, even more preferably BoNT/A1, wherein the clostridial neurotoxin comprises a polypeptide sequence having at least 70% sequence identity to SEQ ID NO: 24. The clostridial neurotoxin may comprise a polypeptide sequence having at least 80% or 90% sequence identity to SEQ ID NO: 24. Preferably the clostridial neurotoxin comprises (or consists of) a polypeptide sequence shown as SEQ ID NO: 24.

The polypeptide sequences of the invention (or the nucleotide sequences encoding the same) may include a purification tag, such as a His-tag. It is intended that the present invention also encompasses polypeptide sequences (and nucleotide sequences encoding the same) where the purification tag is removed.

### Clostridial Neurotoxins

The clostridial neurotoxin (e.g. pre-engineering) may be BoNT/A. An exemplary reference BoNT/A sequence is shown as SEQ ID NO: 25.

The clostridial neurotoxin (e.g. pre-engineering) may be BoNT/B. An exemplary reference BoNT/B sequence is shown as SEQ ID NO: 26.

The clostridial neurotoxin (e.g. pre-engineering) may be BoNT/C. An exemplary reference BoNT/C1 sequence is shown as SEQ ID NO: 27.

The clostridial neurotoxin (e.g. pre-engineering) may be BoNT/D. An exemplary reference BoNT/D sequence is shown as SEQ ID NO: 28.

The clostridial neurotoxin (e.g. pre-engineering) may be BoNT/E. An exemplary reference BoNT/E sequence is shown as SEQ ID NO: 29.

The clostridial neurotoxin (e.g. pre-engineering) may be BoNT/F. An exemplary reference BoNT/F sequence is shown as SEQ ID NO: 30.

The clostridial neurotoxin (e.g. pre-engineering) may be BoNT/G. An exemplary reference BoNT/G sequence is shown as SEQ ID NO: 31.

The clostridial neurotoxin (e.g. pre-engineering) may be BoNT/X. An exemplary reference BoNT/X sequence is shown as SEQ ID NO: 32.

The clostridial neurotoxin (e.g. pre-engineering) may be TeNT. An exemplary reference TeNT sequence is shown as SEQ ID NO: 33.

As discussed above, activated clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

Examples of light chain reference sequences include:
Botulinum type A neurotoxin: amino acid residues 1-448
Botulinum type B neurotoxin: amino acid residues 1-440
Botulinum type C1 neurotoxin: amino acid residues 1-441
Botulinum type D neurotoxin: amino acid residues 1-445
Botulinum type E neurotoxin: amino acid residues 1-422
Botulinum type F neurotoxin: amino acid residues 1-439
Botulinum type G neurotoxin: amino acid residues 1-441
Tetanus neurotoxin: amino acid residues 1-457

For recently-identified BoNT/X, the L-chain has been reported as corresponding to amino acids 1-439 thereof, with the L-chain boundary potentially varying by approximately 25 amino acids (e.g. 1-414 or 1-464).

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441
Botulinum type C1 neurotoxin: amino acid residues M1-K449
Botulinum type D neurotoxin: amino acid residues M1-R445
Botulinum type E neurotoxin: amino acid residues M1-R422
Botulinum type F neurotoxin: amino acid residues M1-K439
Botulinum type G neurotoxin: amino acid residues M1-K446
Tetanus neurotoxin: amino acid residues M1-A457

Alternatively, clostridial neurotoxin L-chains may be defined as the first amino acid (including or excluding an initial methionine residue) through to the first cysteine residue of the endogenous activation loop. In addition or alternatively, a clostridial neurotoxin L-chain may be defined as the amino acid sequence N-terminal to the cleavage site within the endogenous activation loop.

Clostridial neurotoxin L-chains may be defined as a clostridial neurotoxin domain which comprises the metal coordinating HExxH motif (SEQ ID NO: 58), which typically functions to cleave a SNARE protein substrate.

The term “light-chain” (or “L-chain”) encompasses variants and fragments thereof, provided said variants and fragments still demonstrate non-cytotoxic protease activity (which can be determined using standard assays known in the art, examples of which are described herein). By way of example, a variant may have at least 70%, preferably at least 80%, more preferably at least 90%, and most preferably at least 95% or at least 98% amino acid sequence homology with a reference L-chain. The term fragment, when used in relation to a L-chain, means a peptide having at least 200, preferably at least 250, more preferably at least 300, even more preferably at least 350, and most preferably at least 400 amino acid residues of the reference L-chain. In the case of a clostridial L-chain, the fragment preferably at least 300, more preferably at least 350, and most preferably at least 400 amino acid residues of the reference L-chain. L-chain ‘fragments’ of the present invention embrace fragments of variant L-chains based on the reference sequences.

A clostridial neurotoxin H-chains may be defined as the second cysteine of the endogenous activation loop through to the final amino acid. In addition or alternatively, a clostridial neurotoxin H-chain may be defined as starting from the amino acid sequence C-terminal to the cleavage site within the endogenous activation loop. In addition or alternatively, a clostridial neurotoxin H-chain may be defined as starting from the amino acid C-terminal to the cysteine residue (typically the second cysteine residue) that forms a disulphide bond between the L- and H-chain and so defines the C-terminal of the endogenous activation loop.

A Translocation Domain is a molecule that enables translocation of a protease into a target cell such that a functional expression of protease activity occurs within the cytosol of the target cell. Whether any molecule (e.g. a protein or peptide) possesses the requisite translocation function of the present invention may be confirmed by any one of a number of conventional assays.

For example, Shone C. (1987) describes an in vitro assay employing liposomes, which are challenged with a test molecule. Presence of the requisite translocation function is confirmed by release from the liposomes of K⁺ and/or labelled NAD, which may be readily monitored (see Shone C. (1987) Eur. J. Biochem; vol. 167(1): pp. 175-180).

A further example is provided by Blaustein R. (1987), which describes a simple in vitro assay employing planar phospholipid bilayer membranes. The membranes are challenged with a test molecule and the requisite translocation function is confirmed by an increase in conductance across said membranes (see Blaustein (1987) FEBS Letts; vol. 226, no. 1: pp. 115-120).

Additional methodology to enable assessment of membrane fusion and thus identification of Translocation Domains suitable for use in the present invention are provided by Methods in Enzymology Vol 220 and 221, Membrane Fusion Techniques, Parts A and B, Academic Press 1993.

The present invention also embraces variants and/or fragments of translocation domains, so long as the variant domains still demonstrate the requisite translocation activity. By way of example, a variant may have at least 70%, preferably at least 80%, more preferably at least 90%, and most preferably at least 95% or at least 98% amino acid sequence homology with a reference translocation domain. The term fragment, when used in relation to a translocation domain, means a peptide having at least 20, preferably at least 40, more preferably at least 80, and most preferably at least 100 amino acid residues of the reference translocation domain. In the case of a clostridial translocation domain, the fragment preferably has at least 100, preferably at least 150, more preferably at least 200, and most preferably at least 250 amino acid residues of the reference translocation domain (eg. H_{N} domain). Translocation ‘fragments’ of the present invention embrace fragments of variant translocation domains based on the reference sequences.

The Translocation Domain is preferably capable of formation of ion-permeable pores in lipid membranes under conditions of low pH. Preferably it has been found to use only those portions of the protein molecule capable of pore-formation within the endosomal membrane.

The Translocation Domain may be obtained from a microbial protein source, in particular from a bacterial or viral protein source. Hence, the Translocation Domain may be a translocating domain of an enzyme, such as a bacterial toxin or viral protein.

It is well documented that certain domains of bacterial toxin molecules are capable of forming such pores. It is also known that certain translocation domains of virally expressed membrane fusion proteins are capable of forming such pores. Such domains may be employed in the present invention.

The Translocation Domain may be of a clostridial origin, such as the H_{N} domain (or a functional component thereof). H_{N} means a portion or fragment of the H-chain of a clostridial neurotoxin approximately equivalent to the amino-terminal half of the H-chain, or the domain corresponding to that fragment in the intact H-chain. The H_{C} function of the H-chain may be removed by deletion of the H_{C} amino acid sequence (either at the DNA synthesis level, or at the post-synthesis level by nuclease or protease treatment). Alternatively, the H_{C} function may be inactivated by chemical or biological treatment. Thus, the H-chain may be incapable of binding to the Binding Site on a target cell to which native clostridial neurotoxin (i.e. holotoxin) binds.

Examples of suitable (reference) Translocation Domains include:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Botulinum type X neurotoxin—amino acid residues (461-890)
Tetanus neurotoxin—amino acid residues (458-879)

For recently-identified BoNT/X, the translocation domain has been reported as corresponding to amino acids 460-890 thereof, with the L-chain and H_{C} boundaries potentially varying by approximately 10 amino acids (e.g. 461-889 or 454-891).

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)
Botulinum type C neurotoxin—amino acid residues (T450-N866)
Botulinum type D neurotoxin—amino acid residues (D446-N862)
Botulinum type E neurotoxin—amino acid residues (K423-K845)
Botulinum type F neurotoxin—amino acid residues (A440-K864)
Botulinum type G neurotoxin—amino acid residues (S447-S863)
Tetanus neurotoxin—amino acid residues (S458-V879)

In the context of the present invention, a variety of clostridial neurotoxin H_{N} regions comprising a translocation domain can be useful in aspects of the present invention with the proviso that these active fragments can facilitate the release of a non-cytotoxic protease (e.g. a clostridial L-chain) from intracellular vesicles into the cytoplasm of the target cell and thus participate in executing the overall cellular mechanism whereby a clostridial neurotoxin proteolytically cleaves a substrate. The H_{N} regions from the heavy chains of clostridial neurotoxins are approximately 410-430 amino acids in length and comprise a translocation domain. Research has shown that the entire length of a H_{N} region from a clostridial neurotoxin heavy chain is not necessary for the translocating activity of the translocation domain. Thus, in the context of the present invention a translocation domain can include clostridial neurotoxin H_{N} regions comprising a translocation domain having a length of, for example, at least 350 amino acids, at least 375 amino acids, at least 400 amino acids and at least 425 amino acids. Also encompassed are clostridial neurotoxin H_{N} regions comprising translocation domain having a length of, for example, at most 350 amino acids, at most 375 amino acids, at most 400 amino acids and at most 425 amino acids.

For further details on the genetic basis of toxin production in Clostridium botulinum and C. tetani, we refer to Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues, so long as the modified H_{N} portions still demonstrate the above-mentioned translocation function.

Alternatively, the Translocation Domain may be of a non-clostridial origin. Examples of non-clostridial (reference) Translocation Domain origins include, but not be restricted to, the translocation domain of diphtheria toxin (O'Keefe et al., Proc. Natl. Acad. Sci. USA (1992) 89, 6202-6206; Silverman et al., J. Biol. Chem. (1993) 269, 22524-22532; and London, E. (1992) Biochem. Biophys. Acta., 1112, pp.25-51), the translocation domain of Pseudomonas exotoxin type A (Prior et al. Biochemistry (1992) 31, 3555-3559), the translocation domains of anthrax toxin (Blanke et al. Proc. Natl. Acad. Sci. USA (1996) 93, 8437-8442), a variety of fusogenic or hydrophobic peptides of translocating function (Plank et al. J. Biol. Chem. (1994) 269, 12918-12924; and Wagner et al (1992) PNAS, 89, pp.7934-7938), and amphiphilic peptides (Murata et al (1992) Biochem., 31, pp. 1986-1992). The Translocation Domain may mirror the Translocation Domain present in a naturally-occurring protein, or may include amino acid variations so long as the variations do not destroy the translocating ability of the Translocation Domain.

Particular examples of viral (reference) Translocation Domains suitable for use in the present invention include certain translocating domains of virally expressed membrane fusion proteins. For example, Wagner et al. (1992) and Murata et al. (1992) describe the translocation (i.e. membrane fusion and vesiculation) function of a number of fusogenic and amphiphilic peptides derived from the N-terminal region of influenza virus haemagglutinin. Other virally expressed membrane fusion proteins known to have the desired translocating activity are a translocating domain of a fusogenic peptide of Semliki Forest Virus (SFV), a translocating domain of vesicular stomatitis virus (VSV) glycoprotein G, a translocating domain of SER virus F protein and a translocating domain of Foamy virus envelope glycoprotein. Virally encoded Aspike proteins have particular application in the context of the present invention, for example, the E1 protein of SFV and the G protein of the G protein of VSV.

Use of the (reference) Translocation Domains listed in Table (below) includes use of sequence variants thereof. A variant may comprise one or more conservative nucleic acid substitutions and/or nucleic acid deletions or insertions, with the proviso that the variant possesses the requisite translocating function. A variant may also comprise one or more amino acid substitutions and/or amino acid deletions or insertions, so long as the variant possesses the requisite translocating function.

| Translocation | | |
|---|---|---|
| Domain source | Amino acid residues | References |
| Diphtheria toxin | 194-380 | Silverman et al., 1994, J. Biol. Chem. |
| | | 269, 22524-22532 |
| | | London E., 1992, Biochem. Biophys. |
| | | Acta., 1113, 25-51 |
| Domain II of | 405-613 | Prior et al., 1992, Biochemistry 31, 3555- |
| pseudomonas | | 3559 |
| exotoxin | | Kihara & Pastan, 1994, Bioconj Chem. 5, |
| | | 532-538 |
| Influenza virus | GLFGAIAGFIENGWE | Plank et al., 1994, J. Biol. Chem. 269, |
| haemagglutinin | GMIDGWYG (SEQ ID | 12918-12924 |
| | NO: 73), and | Wagner et al., 1992, PNAS, 89, 7934- |
| | Variants thereof | 7938 |
| | | Murata et al., 1992, Biochemistry 31, |
| | | 1986-1992 |
| Semliki Forest virus | Translocation domain | Kielian et al., 1996, J Cell Biol. 134(4), |
| fusogenic protein | | 863-872 |
| Vesicular Stomatitis | 118-139 | Yao et al., 2003, Virology 310(2), 319-332 |
| virus glycoprotein | | |
| G | | |
| SER virus F protein | Translocation domain | Seth et al., 2003, J Virol 77(11) 6520- |
| | | 6527 |
| Foamy virus | Translocation domain | Picard-Maureau et al., 2003, J Virol. |
| envelope | | 77(8), 4722-4730 |
| glycoprotein | | |

Examples of clostridial neurotoxin H_{C} domain reference sequences include:
BONT/A—N872-L1296
BONT/B—E859-E1291
BONT/C1—N867-E1291
BONT/D—S863-E1276
BONT/E—R846-K1252
BONT/F—K865-E1274
BONT/G—N864-E1297
TeNT—I880-D1315

For recently-identified BoNT/X, the H_{C} domain has been reported as corresponding to amino acids 893-1306 thereof, with the domain boundary potentially varying by approximately 25 amino acids (e.g. 868-1306 or 918-1306).

The clostridial neurotoxins described herein may further comprise a translocation facilitating domain. Said domain facilitates delivery of the non-cytotoxic protease into the cytosol of the target cell and are described, for example, in WO 08/008803 and WO 08/008805, each of which is herein incorporated by reference thereto.

By way of example, suitable translocation facilitating domains include an enveloped virus fusogenic peptide domain, for example, suitable fusogenic peptide domains include influenzavirus fusogenic peptide domain (eg. influenza A virus fusogenic peptide domain of 23 amino acids), alphavirus fusogenic peptide domain (eg. Semliki Forest virus fusogenic peptide domain of 26 amino acids), vesiculovirus fusogenic peptide domain (eg. vesicular stomatitis virus fusogenic peptide domain of 21 amino acids), respirovirus fusogenic peptide domain (eg. Sendai virus fusogenic peptide domain of 25 amino acids), morbiliivirus fusogenic peptide domain (eg. Canine distemper virus fusogenic peptide domain of 25 amino acids), avulavirus fusogenic peptide domain (eg. Newcastle disease virus fusogenic peptide domain of 25 amino acids), henipavirus fusogenic peptide domain (eg. Hendra virus fusogenic peptide domain of 25 amino acids), metapneumovirus fusogenic peptide domain (eg. Human metapneumovirus fusogenic peptide domain of 25 amino acids) or spumavirus fusogenic peptide domain such as simian foamy virus fusogenic peptide domain; or fragments or variants thereof.

By way of further example, a translocation facilitating domain may comprise a clostridial neurotoxin H_{CN} domain or a fragment or variant thereof. In more detail, a clostridial neurotoxin H_{CN} translocation facilitating domain may have a length of at least 200 amino acids, at least 225 amino acids, at least 250 amino acids, at least 275 amino acids. In this regard, a clostridial neurotoxin H_{CN} translocation facilitating domain preferably has a length of at most 200 amino acids, at most 225 amino acids, at most 250 amino acids, or at most 275 amino acids. Specific (reference) examples include:
Botulinum type A neurotoxin—amino acid residues (872-1110)
Botulinum type B neurotoxin—amino acid residues (859-1097)
Botulinum type C neurotoxin—amino acid residues (867-1111)
Botulinum type D neurotoxin—amino acid residues (863-1098)
Botulinum type E neurotoxin—amino acid residues (846-1085)
Botulinum type F neurotoxin—amino acid residues (865-1105)
Botulinum type G neurotoxin—amino acid residues (864-1105)
Botulinum type X neurotoxin—amino acid residues (890-1121)
Tetanus neurotoxin—amino acid residues (880-1127)

The above sequence positions may vary a little according to serotype/sub-type, and further examples of suitable (reference) clostridial neurotoxin H_{CN} domains include:
Botulinum type A neurotoxin—amino acid residues (874-1110)
Botulinum type B neurotoxin—amino acid residues (861-1097)
Botulinum type C neurotoxin—amino acid residues (869-1111)
Botulinum type D neurotoxin—amino acid residues (865-1098)
Botulinum type E neurotoxin—amino acid residues (848-1085)
Botulinum type F neurotoxin—amino acid residues (867-1105)
Botulinum type G neurotoxin—amino acid residues (866-1105)
Tetanus neurotoxin—amino acid residues (882-1127)

Any of the above-described facilitating domains may be combined with any of the previously described translocation domain peptides that are suitable for use in the present invention. Thus, by way of example, a non-clostridial facilitating domain may be combined with non-clostridial translocation domain peptide or with clostridial translocation domain peptide. Alternatively, a clostridial neurotoxin H_{CN} translocation facilitating domain may be combined with a non-clostridial translocation domain peptide. Alternatively, a clostridial neurotoxin HCN facilitating domain may be combined or with a clostridial translocation domain peptide, examples of which include:
Botulinum type A neurotoxin—amino acid residues (449-1110)
Botulinum type B neurotoxin—amino acid residues (442-1097)
Botulinum type C neurotoxin—amino acid residues (450-1111)
Botulinum type D neurotoxin—amino acid residues (446-1098)
Botulinum type E neurotoxin—amino acid residues (423-1085)
Botulinum type F neurotoxin—amino acid residues (440-1105)
Botulinum type G neurotoxin—amino acid residues (447-1105)
Tetanus neurotoxin—amino acid residues (458-1127)

In some embodiments the clostridial neurotoxins of the present invention may lack a functional H_{C} domain of a clostridial neurotoxin. Accordingly, said clostridial neurotoxins are not able to bind rat synaptosomal membranes (via a clostridial H_{C} component) in binding assays as described in Shone et al. (1985) Eur. J. Biochem. 151, 75-82. The clostridial neurotoxins may preferably lack the last 50 C-terminal amino acids of a clostridial neurotoxin holotoxin. The clostridial neurotoxins may preferably lack the last 100, preferably the last 150, more preferably the last 200, particularly preferably the last 250, and most preferably the last 300 C-terminal amino acid residues of a clostridial neurotoxin holotoxin. Alternatively, the H_{C} binding activity may be negated/reduced by mutagenesis—by way of example, referring to BoNT/A for convenience, modification of one or two amino acid residue mutations (W1266 to L and Y1267 to F) in the ganglioside binding pocket causes the H_{C} region to lose its receptor binding function. Analogous mutations may be made to non-serotype A clostridial peptide components, e.g. a construct based on botulinum B with mutations (W1262 to L and Y1263 to F) or botulinum E (W1224 to L and Y1225 to F). Other mutations to the active site achieve the same ablation of H_{C} receptor binding activity, e.g. Y1267S in botulinum type A toxin and the corresponding highly conserved residue in the other clostridial neurotoxins. Details of this and other mutations are described in Rummel et al (2004) (Molecular Microbiol. 51:631-634), which is hereby incorporated by reference thereto.

The H_{C} peptide of a native clostridial neurotoxin comprises approximately 400-440 amino acid residues, and consists of two functionally distinct domains of approximately 25 kDa each, namely the N-terminal region (commonly referred to as the H_{CN} peptide or domain) and the C-terminal region (commonly referred to as the Hcc peptide or domain). This fact is confirmed by the following publications, each of which is herein incorporated in its entirety by reference thereto: Umland TC (1997) Nat. Struct. Biol. 4:788-792; Herreros J (2000) Biochem. J. 347:199-204; Halpern J (1993) J. Biol. Chem. 268:15, pp. 11188-11192; Rummel A (2007) PNAS 104:359-364; Lacey DB (1998) Nat. Struct. Biol. 5:898-902; Knapp (1998) Am. Cryst. Assoc. Abstract Papers 25:90; Swaminathan and Eswaramoorthy (2000) Nat. Struct. Biol. 7:1751-1759; and Rummel A (2004) Mol. Microbiol. 51(3), 631-643. Moreover, it has been well documented that the C-terminal region (Hcc), which constitutes the C-terminal 160-200 amino acid residues, is responsible for binding of a clostridial neurotoxin to its natural cell receptors, namely to nerve terminals at the neuromuscular junction—this fact is also confirmed by the above publications. Thus, reference throughout this specification to a clostridial heavy-chain lacking a functional heavy chain H_{C} peptide (or domain) such that the heavy-chain is incapable of binding to cell surface receptors to which a native clostridial neurotoxin binds means that the clostridial heavy-chain simply lacks a functional H_{CC} peptide. In other words, the H_{CC} peptide region may be either partially or wholly deleted, or otherwise modified (e.g. through conventional chemical or proteolytic treatment) to inactivate its native binding ability for nerve terminals at the neuromuscular junction. Thus, a clostridial neurotoxin H_{N} peptide of the present invention may be C-terminally extended, i.e. it may be associated with all or part of a clostridial neurotoxin H_{C} domain, e.g. the H_{CN}, H_{CC} or H_{C} domain. References herein to a clostridial neurotoxin H_{N} peptide of the present invention encompass such C-terminally extended H_{N} peptides, which comprise one or more amino acid residues from a clostridial neurotoxin H_{C} domain. Alternatively, a clostridial neurotoxin H_{N} peptide of the present invention may not be associated with (or lack) all or part of a clostridial neurotoxin H_{C} domain, e.g. the H_{CN}, H_{CC} or H_{C} domain.

Typically if a clostridial neurotoxin of the invention or a clostridial neurotoxin H_{N} peptide of the present invention lacks all or part of a C-terminal peptide portion (H_{CC}) of a clostridial neurotoxin it thus lacks the H_{C} binding function of native clostridial neurotoxin. By way of example, a C-terminally extended clostridial H_{N} peptide may lack the C-terminal 40 amino acid residues, or the C-terminal 60 amino acid residues, or the C-terminal 80 amino acid residues, or the C-terminal 100 amino acid residues, or the C-terminal 120 amino acid residues, or the C-terminal 140 amino acid residues, or the C-terminal 150 amino acid residues, or the C-terminal 160 amino acid residues of a clostridial neurotoxin heavy-chain. Alternatively, the clostridial H_{N} peptide of the present invention may lack the entire C-terminal peptide portion (H_{CC}) of a clostridial neurotoxin and thus lacks the H_{C} binding function of native clostridial neurotoxin. By way of example, the clostridial H_{N} peptide may lack the C-terminal 165 amino acid residues, or the C-terminal 170 amino acid residues, or the C-terminal 175 amino acid residues, or the C-terminal 180 amino acid residues, or the C-terminal 185 amino acid residues, or the C-terminal 190 amino acid residues, or the C-terminal 195 amino acid residues of a clostridial neurotoxin heavy-chain. By way of further example, the clostridial H_{N} peptide of the present invention lacks a clostridial H_{CC} reference sequence selected from the group consisting of:
Botulinum type A neurotoxin—amino acid residues (Y1111-L1296)
Botulinum type B neurotoxin—amino acid residues (Y1098-E1291)
Botulinum type C neurotoxin—amino acid residues (Y1112-E1291)
Botulinum type D neurotoxin—amino acid residues (Y1099-E1276)
Botulinum type E neurotoxin—amino acid residues (Y1086-K1252)
Botulinum type F neurotoxin—amino acid residues (Y1106-E1274)
Botulinum type G neurotoxin—amino acid residues (Y1106-E1297)
Botulinum type X neurotoxin—amino acid residues (Y1122-D1306) Tetanus neurotoxin—amino acid residues (Y1128-D1315).

The above-identified reference sequences should be considered a guide as slight variations may occur according to sub-serotypes.

The present invention is suitable for application to many different varieties of clostridial neurotoxin. Thus, in the context of the present invention, the term “clostridial neurotoxin” embraces toxins produced by C. botulinum (botulinum neurotoxin serotypes A, B, C1, D, E, F, G, H, and X), C. tetani (tetanus neurotoxin), C. butyricum (botulinum neurotoxin serotype E), and C. baratii (botulinum neurotoxin serotype F), as well as modified clostridial neurotoxins or derivatives derived from any of the foregoing. The term “clostridial neurotoxin” also embraces botulinum neurotoxin serotype H. Preferably the clostridial neurotoxin is BoNT/A, more preferably BoNT/A₁.

Botulinum neurotoxin (BoNT) is produced by C. botulinum in the form of a large protein complex, consisting of BoNT itself complexed to a number of accessory proteins. There are at present nine different classes of botulinum neurotoxin, namely: botulinum neurotoxin serotypes A, B, C1, D, E, F, G, H, and X all of which share similar structures and modes of action. Different BONT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1.

Tetanus toxin is produced in a single serotype by C. tetani. C. butyricum produces BONT/E, while C. baratii produces BoNT/F.

The term “clostridial neurotoxin” is also intended to embrace modified clostridial neurotoxins and derivatives thereof, including but not limited to those described below. A modified clostridial neurotoxin or derivative may contain one or more amino acids that has been modified as compared to the native (unmodified) form of the clostridial neurotoxin, or may contain one or more inserted amino acids that are not present in the native (unmodified) form of the clostridial neurotoxin. By way of example, a modified clostridial neurotoxin may have modified amino acid sequences in one or more domains relative to the native (unmodified) clostridial neurotoxin sequence. Such modifications may modify functional aspects of the toxin, for example biological activity or persistence. Thus, a clostridial neurotoxin of the invention may be a modified clostridial neurotoxin, or a modified clostridial neurotoxin derivative, or a clostridial neurotoxin derivative. In particular, an engineered clostridial neurotoxin of the invention may be an engineered modified clostridial neurotoxin, or an engineered modified clostridial neurotoxin derivative, or an engineered clostridial neurotoxin derivative.

A modified clostridial neurotoxin may have one or more modifications in the amino acid sequence of the heavy chain (such as a modified H_{C} domain), wherein said modified heavy chain binds to target nerve cells with a higher or lower affinity than the native (unmodified) clostridial neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

A modified clostridial neurotoxin may have one or more modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more modifications that increases or decreases the biological activity and/or the biological persistence of the modified clostridial neurotoxin. For example, a modified clostridial neurotoxin may comprise a leucine- or tyrosine-based motif, wherein said motif increases or decreases the biological activity and/or the biological persistence of the modified clostridial neurotoxin. Suitable leucine-based motifs include xDxxxLL (SEQ ID NO: 60), xExxxLL (SEQ ID NO: 61), xExxxIL (SEQ ID NO: 62), and xExxxLM (SEQ ID NO: 63) (wherein x is any amino acid). Suitable tyrosine-based motifs include Y-x-x-Hy (SEQ ID NO: 64) (wherein Hy is a hydrophobic amino acid). Examples of modified clostridial neurotoxins comprising leucine- and tyrosine-based motifs are described in WO 2002/008268, which is hereby incorporated by reference in its entirety.

The term “clostridial neurotoxin” is intended to embrace hybrid and chimeric clostridial neurotoxins. A hybrid clostridial neurotoxin comprises at least a portion of a light chain from one clostridial neurotoxin or subtype thereof, and at least a portion of a heavy chain from another clostridial neurotoxin or clostridial neurotoxin subtype. A hybrid clostridial neurotoxin may contain the entire light chain from one clostridial neurotoxin subtype and the heavy chain from another clostridial neurotoxin subtype. A chimeric clostridial neurotoxin may contain a portion (e.g. the binding domain) of the heavy chain of one clostridial neurotoxin subtype, with another portion of the heavy chain being from another clostridial neurotoxin subtype. A chimeric clostridial neurotoxin, particularly a chimeric BoNT, may be defined in terms of the serotype or sub-serotype of the four main domains of the neurotoxin: L-chain, H_{N}, H_{CN} and H_{CC} (as defined herein). For example, the (pre-engineering) LH_{N}/A1-H_{C}B1 chimera of SEQ ID NO: 69 may be described as an AABB chimera. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to patients who are immunologically resistant to a given clostridial neurotoxin subtype, to patients who may have a lower than average concentration of receptors to a given clostridial neurotoxin heavy chain binding domain, or to patients who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety. Thus, a clostridial neurotoxin of the invention may be a hybrid clostridial neurotoxin, or a chimeric clostridial neurotoxin. In particular, an engineered clostridial neurotoxin of the invention may be an engineered hybrid clostridial neurotoxin, or an engineered chimeric clostridial neurotoxin.

In some preferred embodiments, a clostridial neurotoxin is BoNT/A comprising at least one domain from a non-BoNT/A clostridial neurotoxin (e.g. a BoNT/A hybrid or chimera). For example, a clostridial neurotoxin of the invention (comprising a furin cleavage site) may comprise:
i. A BONT/A L-chain and a non-BoNT/A H_{N} and H_{C} domain;
ii. A BONT/A H_{N} domain and a non-BoNT/A L-chain and H_{C} domain
iii. A BONT/A H_{C} domain and a non-BoNT/A L-chain and H_{N} domain;
iv. A BONT/A L-chain and H_{N} domain and a non-BoNT/A H_{C} domain
v. A BONT/A L-chain and H_{C} domain and a non-BoNT/A H_{N} domain; or
vi. A BONT/A H_{N} domain and H_{C} domain and a non-BoNT/A L-chain.

By way of non-limiting example, a clostridial neurotoxin of the invention (e.g. an engineered clostridial neurotoxin) comprises a BoNT/A L-chain and H_{N} domain and a BoNT/B H_{C} domain (such as LH_{N}/A1-H_{C}/B1). An exemplary non-engineered LH_{N}/A1-H_{C}B1 chimera that may be modified to comprises a furin cleavage site according to the invention is given in SEQ ID NO: 69. An exemplary engineered form of the LH_{N}/A1-H_{C}B1 chimera of SEQ ID NO: 69 is given in SEQ ID NO: 70. A clostridial neurotoxin of the invention (e.g. an engineered clostridial neurotoxin) may comprise a BoNT/A L-chain and H_{N} domain and a BoNT/C1 H_{C} domain. A clostridial neurotoxin of the invention (e.g. an engineered clostridial neurotoxin) may comprise a BoNT/A L-chain and H_{N} domain and a BoNT/D H_{C} domain. A clostridial neurotoxin of the invention (e.g. an engineered clostridial neurotoxin) may comprise a BoNT/A L-chain and H_{N} domain and a BoNT/E H_{C} domain. A clostridial neurotoxin of the invention (e.g. an engineered clostridial neurotoxin) may comprise a BoNT/A L-chain and H_{N} domain and a BoNT/F H_{C} domain. A clostridial neurotoxin of the invention (e.g. an engineered clostridial neurotoxin) may comprise a BoNT/A L-chain and H_{N} domain and a BoNT/G H_{C} domain. A clostridial neurotoxin of the invention (e.g. an engineered clostridial neurotoxin) may comprise a BoNT/A L-chain and H_{N} domain and a BoNT/X H_{C} domain. A clostridial neurotoxin of the invention (e.g. an engineered clostridial neurotoxin) may comprise a BoNT/A L-chain and H_{N} domain and a TeNT H_{C} domain.

For example, a clostridial neurotoxin of the invention (e.g. an engineered clostridial
i. A BONT/B L-chain and a non-BoNT/B H_{N} and H_{C} domain;
ii. A BONT/B H_{N} domain and a non-BoNT/B L-chain and H_{C} domain
iii. A BONT/B H_{C} domain and a non-BoNT/B L-chain and H_{N} domain;
iv. A BONT/B L-chain and H_{N} domain and a non-BoNT/B H_{C} domain
v. A BONT/B L-chain and H_{C} domain and a non-BoNT/B H_{N} domain; or
vi. A BONT/B H_{N} domain and H_{C} domain and a non-BoNT/B L-chain. For example, a clostridial neurotoxin of the invention (e.g. an engineered clostridial neurotoxin comprising a furin cleavage site) may comprise:
i. A BoNT/C1 L-chain and a non-BoNT/C1 H_{N} and H_{C} domain;
ii. A BONT/C1 H_{N} domain and a non-BoNT/C1 L-chain and H_{C} domain
iii. A BONT/C1 H_{C} domain and a non-BoNT/C1 L-chain and H_{N} domain;
iv. A BONT/C1 L-chain and H_{N} domain and a non-BoNT/C1 H_{C} domain
v. A BONT/C1 L-chain and H_{C} domain and a non-BoNT/C1 H_{N} domain; or
vi. A BONT/C1 H_{N} domain and H_{C} domain and a non-BoNT/C1 L-chain.

Non-limiting examples include BoNT/C1 chimeras where the non-BoNT/C1 element is from a BoNT/D (i.e. BoNT/CD chimeras).

For example, a clostridial neurotoxin of the invention (e.g. an engineered clostridial neurotoxin comprising a furin cleavage site) may comprise:
i. A BONT/D L-chain and a non-BoNT/D H_{N} and H_{C} domain;
ii. A BONT/D H_{N} domain and a non-BoNT/D L-chain and H_{C} domain
iii. A BONT/D H_{C} domain and a non-BoNT/D L-chain and H_{N} domain;
iv. A BONT/D L-chain and H_{N} domain and a non-BoNT/D H_{C} domain
v. A BONT/D L-chain and H_{C} domain and a non-BoNT/D H_{N} domain; or
vi. A BONT/D H_{N} domain and H_{C} domain and a non-BoNT/D L-chain.

Non-limiting examples include BoNT/D chimeras where the non-BoNT/D element is from BoNT/C1 (i.e. BoNT/DC1 chimeras).

For example, a clostridial neurotoxin of the invention (e.g. an engineered clostridial neurotoxin comprising a furin cleavage site) may comprise:
i. A BONT/E L-chain and a non-BoNT/E H_{N} and H_{C} domain;
ii. A BONT/E H_{N} domain and a non-BoNT/E L-chain and H_{C} domain
iii. A BONT/E H_{C} domain and a non-BoNT/E L-chain and H_{N} domain;
iv. A BONT/E L-chain and H_{N} domain and a non-BoNT/E H_{C} domain
v. A BONT/E L-chain and H_{C} domain and a non-BoNT/E H_{N} domain; or
vi. A BONT/E H_{N} domain and H_{C} domain and a non-BoNT/E L-chain.

For example, a clostridial neurotoxin of the invention (e.g. an engineered clostridial
i. A BONT/F L-chain and a non-BoNT/F H_{N} and H_{C} domain;
ii. A BONT/F H_{N} domain and a non-BoNT/F L-chain and H_{C} domain
iii. A BoNT/F H_{C} domain and a non-BoNT/F L-chain and H_{N} domain;
iv. A BONT/F L-chain and H_{N} domain and a non-BoNT/F H_{C} domain
v. A BONT/F L-chain and H_{C} domain and a non-BoNT/F H_{N} domain; or
vi. A BONT/F H_{N} domain and H_{C} domain and a non-BoNT/F L-chain.

For example, a clostridial neurotoxin of the invention (e.g. an engineered clostridial neurotoxin comprising a furin cleavage site) may comprise:
i. A BONT/G L-chain and a non-BoNT/G H_{N} and H_{C} domain;
ii. A BONT/G H_{N} domain and a non-BoNT/G L-chain and H_{C} domain
iii. A BoNT/G H_{C} domain and a non-BoNT/G L-chain and H_{N} domain;
iv. A BONT/G L-chain and H_{N} domain and a non-BoNT/G H_{C} domain
v. A BoNT/G L-chain and H_{C} domain and a non-BoNT/G H_{N} domain; or
vi. A BONT/G H_{N} domain and H_{C} domain and a non-BoNT/G L-chain.

For example, a clostridial neurotoxin of the invention (e.g. an engineered clostridial neurotoxin comprising a furin cleavage site) may comprise:
i. A BONT/X L-chain and a non-BoNT/X H_{N} and H_{C} domain;
ii. A BONT/X H_{N} domain and a non-BoNT/X L-chain and H_{C} domain
iii. A BONT/X H_{C} domain and a non-BoNT/X L-chain and H_{N} domain;
iv. A BONT/X L-chain and H_{N} domain and a non-BoNT/X H_{C} domain
v. A BONT/X L-chain and H_{C} domain and a non-BoNT/X H_{N} domain; or
vi. A BONT/X H_{N} domain and H_{C} domain and a non-BoNT/X L-chain.

For example, a clostridial neurotoxin of the invention (e.g. an engineered clostridial neurotoxin comprising a furin cleavage site) may comprise:
i. A TeNT L-chain and a non-TeNT H_{N} and H_{C} domain;
ii. A TeNT H_{N} domain and a non-TeNT L-chain and H_{C} domain
iii. A TeNT H_{C} domain and a non-TeNT L-chain and H_{N} domain;
iv. A TeNT L-chain and H_{N} domain and a non-TeNT H_{C} domain
v. A TeNT L-chain and H_{C} domain and a non-TeNT H_{N} domain; or
vi. A TeNT H_{N} domain and H_{C} domain and a non-TeNT L-chain.

The term “clostridial neurotoxin” may also embrace newly discovered botulinum neurotoxin and botulinum neurotoxin-like protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OTO22244.1), which cleaves VAMP2 and SNAP25, the Chryseobacterium pipero encoded toxin (NCBI Ref. Seq: WP_034687872.1) and the mosquito BoNT-like protein PMP1 (NCBI Ref. Seq: QEZ70852.1).

The term “clostridial neurotoxin” is intended to embrace re-targeted clostridial neurotoxins. In a re-targeted clostridial neurotoxin, the clostridial neurotoxin is modified to include an exogenous ligand (i.e. not derived from a clostridial neurotoxin) known as a Targeting Moiety (TM). The TM is selected to provide binding specificity for a desired target cell, and as part of the re-targeting process the native binding portion of the clostridial neurotoxin (e.g. the H_{C} domain, or the H_{CC} domain) may be removed. Re-targeting technology is described, for example, in: EP-B-0689459; WO 1994/021300; EP-B-0939818; U.S. Pat. Nos. 6,461,617; 7,192,596; WO 1998/007864; EP-B-0826051; U.S. Pat. Nos. 5,989,545; 6,395,513; 6,962,703; WO 1996/033273; EP-B-0996468; U.S. Pat. No. 7,052,702; WO 1999/017806; EP-B-1107794; U.S. Pat. No. 6,632,440; WO 2000/010598; WO 2001/21213; WO 2006/059093; WO 2000/62814; WO 2000/04926; WO 1993/15766; WO 2000/61192; and WO 1999/58571; all of which are hereby incorporated by reference in their entirety. Thus, a clostridial neurotoxin of the invention may be a re-targeted clostridial neurotoxin. In particular, an engineered clostridial neurotoxin of the invention may be an engineered re-targeted clostridial neurotoxin. The engineered re-targeted clostridial neurotoxins of the invention may comprise TM that are presented at the N- or C-terminus of the single-chain neurotoxin, or the TM may be presented centrally within the single-chain neurotoxin. In some preferred embodiments, the engineered re-targeted clostridial neurotoxins of the invention may comprise TM that are presented at the N- or C-terminus of the single-chain neurotoxin.

Engineering re-targeted clostridial neurotoxins may allow for the use of TM that are susceptible to cleavage by proteases conventionally used to activate recombinantly produced re-targeted clostridial neurotoxins, such as trypsin, Lys-C and/or BoNT hydrolase. Thus, engineering re-targeted clostridial neurotoxins to include a furin activation site according to the invention may allow for improvements in stability compared to a corresponding re-targeted clostridial neurotoxins which is activated by a conventional activating protease such as Lys-C, trypsin and/or BoNT hydrolase.

The clostridial neurotoxin of the present invention (e.g. an engineered clostridial neurotoxin) may lack a functional H_{C} domain of a clostridial neurotoxin and also lack any functionally equivalent TM. Accordingly, said polypeptides lack the natural binding function of a clostridial neurotoxin and are not able to bind rat synaptosomal membranes (via a clostridial H_{C} component, or via any functionally equivalent TM) in binding assays as described in Shone et al. (1985) Eur. J. Biochem. 151, 75-82. Preferably the TM is not a Wheat Germ Agglutinin (WGA) peptide. Thus, in some preferred embodiments the clostridial neurotoxin is a re-targeted clostridial neurotoxin in which an endogenous H_{C} or H_{CC} of a clostridial neurotoxin is replaced by an exogenous TM. Particularly preferred are embodiments in which the engineered clostridial neurotoxin is a re-targeted clostridial neurotoxin in which an endogenous H_{C} or H_{CC} of a clostridial neurotoxin is replaced by an exogenous TM.

A clostridial neurotoxin of the invention (e.g. an engineered clostridial neurotoxin) may comprise an LH_{N} polypeptide (e.g. an engineered LH_{N} polypeptide), i.e. a polypeptide comprising or consisting of a clostridial L-chain and a clostridial H_{N} domain, as defined herein.

A clostridial neurotoxin (e.g. an engineered clostridial neurotoxin) may comprise an LH_{N} polypeptide (e.g. an engineered LH_{N} polypeptide) and a targeting moiety (TM).

The present invention also embraces clostridial neurotoxins that have an additional non-native protease cleavage site. Such a site will require an exogenous protease for cleavage, which allows for improved control over the timing and location of cleavage events. Non-native protease cleavage sites that may be employed in clostridial neurotoxins include:

TEV(Tobacco Etch virus)
(SEQ ID NO: 65)
(ENLYFQ↓G)
Thrombin
(SEQ ID NO: 66)
(LVPR↓GS)
PreScission
(SEQ ID NO: 67)
(LEVLFQ↓GP).

Additional protease cleavage sites include recognition sequences that are cleaved by a non-cytotoxic protease, for example by the light chain of a clostridial neurotoxin. These include the SNARE (e.g. SNAP-25, syntaxin, VAMP) protein recognition sequences that are cleaved by non-cytotoxic proteases such as the light chain of a clostridial neurotoxin. Clostridial neurotoxins comprising non-native protease cleavage sites are described in U.S. Pat. No. 7,132,259, EP 1206554-B2 and US 2007/0166332, all of which are hereby incorporated by reference in their entirety. Also embraced by the term protease cleavage site is an intein, which is a self-cleaving sequence. The self-splicing reaction is controllable, for example by varying the concentration of reducing agent present.

The present invention also embraces clostridial neurotoxins comprising a “destructive cleavage site”. In said clostridial neurotoxins, a non-native protease cleavage site is incorporated into the clostridial neurotoxin, at a location chosen such that cleavage at said site will decrease the activity of, or inactivate, the clostridial neurotoxin. The destructive protease cleavage site can be susceptible to cleavage by a local protease, in the event that the clostridial neurotoxin, following administration, migrates to a non-target location. Suitable non-native protease cleavage sites include those described above. Clostridial neurotoxins comprising a destructive cleavage site are described in WO 2010/094905 and WO 2002/044199, both of which are hereby incorporated by reference in their entirety.

The clostridial neurotoxins (e.g. engineered clostridial neurotoxins) of the present invention, especially the light chain component thereof, may be PEGylated—this may help to increase stability, for example duration of action of the light chain component. PEGylation is particularly preferred when the light chain comprises a BoNT/A, B or C1 protease. PEGylation preferably includes the addition of PEG to the N-terminus of the light chain component. By way of example, the N-terminus of a light chain may be extended with one or more amino acid (e.g. cysteine) residues, which may be the same or different. One or more of said amino acid residues may have its own PEG molecule attached (e.g. covalently attached) thereto. An example of this technology is described in WO2007/104567, which is hereby incorporated by reference in its entirety.

The clostridial neurotoxins (e.g. engineered clostridial neurotoxins) of the present invention may be free from the complexing proteins that are present in a naturally occurring clostridial neurotoxin complex.

The clostridial neurotoxins (e.g. engineered clostridial neurotoxins) of the present invention can be produced using recombinant nucleic acid technologies. Thus, in an engineered clostridial neurotoxin (as described above) may be a recombinant engineered clostridial neurotoxin. A single-chain clostridial neurotoxin (as described herein) may be a recombinant single-chain neurotoxin.

Tolerance to an engineered clostridial neurotoxin of the invention may be increased compared with the tolerance to the corresponding (pre-engineering) clostridial neurotoxin. In particular, tolerance to an engineered clostridial neurotoxin of the invention may be increased compared with the tolerance to the corresponding (pre-engineering) clostridial neurotoxin when the pre-engineering clostridial neurotoxin is administered (e.g. in di-chain form).

An engineered clostridial neurotoxin of the invention may have equivalent or increased potency compared with the potency of the corresponding (pre-engineering) clostridial neurotoxin. In particular, potency of an engineered clostridial neurotoxin of the invention may be equivalent to or increased compared with the potency of the corresponding (pre-engineering) clostridial neurotoxin when the pre-engineering clostridial neurotoxin is administered in di-chain form. The term “equivalent potency” as used herein means that an engineered clostridial neurotoxin has a potency of at least about 80%, at least about 85%, at least about 90%, at least about 95%, at least about 99%, up to about 100% of the potency of the corresponding (pre-engineering) clostridial neurotoxin. Preferably “equivalent potency” as used herein means that an engineered clostridial neurotoxin has a potency of at least about 95%, at least about 99%, at least about 100%, at least about 101%, up to about 105% of the potency of the corresponding (pre-engineering) clostridial neurotoxin. The term “increased potency” as used herein means that an engineered clostridial neurotoxin has a potency of at least about 10%, at least about 15%, at least about 20%, at least about 25% greater potency compared with the potency of the corresponding (pre-engineering) clostridial neurotoxin. Potency may be measured using any appropriate assay, conventional examples of which are described herein.

An engineered clostridial neurotoxin of the invention typically has an improved safety profile and/or therapeutic window compared with the safety profile and/or therapeutic window of the corresponding (pre-engineering) clostridial neurotoxin. Without being bound by theory, this may be by virtue of its improved tolerance and/or equivalent or increased potency. In particular, an engineered clostridial neurotoxin of the invention may have an improved safety profile and/or therapeutic window compared with the safety profile and/or therapeutic window of the corresponding (pre-engineering) clostridial neurotoxin when the pre-engineering clostridial neurotoxin is administered (e.g. in di-chain form).

One way in which these advantageous properties (which represent an increase in the therapeutic index) may be defined is in terms of the Safety Ratio (for clinical applications) or Tolerance Index (TI, in animal models) of the engineered clostridial toxin. In this regard, undesired effects of a clostridial neurotoxin (such as those caused by diffusion of the neurotoxin away from the site of administration) can be assessed experimentally by measuring percentage bodyweight loss in a relevant animal model (e.g. a mouse, where loss of bodyweight is detected within seven days of administration). Desired on-target effects of a clostridial toxin can be assessed experimentally by any appropriate technique, depending on the target cell of interest. Suitable assays are known in the art and it would be routine for one of ordinary skill to select an appropriate assay for a given target cell type. For clostridial neurotoxins of the invention which target motor neurons, a Digital Abduction Score (DAS) assay, a measurement of muscle paralysis, may be used. The DAS assay may be performed by injection of 20 μl of (engineered) clostridial toxin, formulated in Gelatin Phosphate Buffer, into the mouse gastrocnemius/soleus complex, followed by assessment of Digital Abduction Score using the method of Aoki (Aoki KR, Toxicon 39:1815-1820; 2001). In the DAS assay, mice are suspended briefly by the tail in order to elicit a characteristic startle response in which the mouse extends its hind limbs and abducts its hind digits. Following clostridial toxin injection, the varying degrees of digit abduction are scored on a five-point scale (0=normal to 4=maximal reduction in digit abduction and leg extension). For clostridial neurotoxins of the invention which target other subtypes of neurones, any appropriate assay known in the art may be used. SNARE cleavage assays may also be used to assess the activity of engineered clostridial neurotoxins of the invention, examples of which are well-described in the art (e.g. Western blot). Assays to detect and/or quantify the effect of an engineered clostridial neurotoxin on the release of a maker signalling molecule may also be used. The specific marker signally molecule may be selected depending on the cell type(s) targeted by the engineered clostridial neurotoxins. For example, the signalling molecule may be a hormone, substance P, CGRP, glutamate, glycine, depending on whether cells involved with hormone secretion or pain-sensing neurons are targeted. For the treatment of pain, animal studies may be used to assess if there is a greater tolerance to a noxious stimulus. Typical in vivo assays will measure different types of pain (e.g., mechanical, cold, heat) and the readout could be behavioural (e.g., licking/biting the treated site or withdrawal from the noxious stimulus) or may involve the use of the Von Frey test. Any appropriate nociception test may be used, and examples of such tests are well-known in the art.

The Safety Ratio or TI of a clostridial neurotoxin may then be expressed as the ratio between the amount of toxin required for a 10% drop in a bodyweight (measured at peak effect within the first seven days after dosing in a mouse) and the amount of toxin required for a DAS score of 2. High Safety Ratio or TI scores are therefore desired, and indicate a toxin that is able to effectively paralyse a target muscle with little undesired off-target effects. An engineered toxin of the present invention may have a Safety Ratio and/or TI that is higher than the Safety Ratio and/or TI of an equivalent unmodified (pre-engineering) single-chain clostridial neurotoxin. The calculation for TI may vary depending on the experimental model used.

For example, in a DAS mouse model, an engineered clostridial toxin of the present invention has a TI of at least 8 (for example, at least 8, 9, 10, 15, 20, 25, 30, 35, 40, 45 or 50), wherein TI is calculated as: dose of toxin required for −10% bodyweight change (pg/mouse) divided by DAS ED₅₀ (pg/mouse) [ED₅₀=dose required to produce a DAS score of 2].

For clinical use a Safety Ratio may be calculated.

The invention provides a nucleic acid (for example, a DNA or RNA) comprising a nucleic acid sequence encoding a clostridial neurotoxin (e.g. an engineered clostridial neurotoxin) as described herein. The nucleic acid sequence may be prepared as part of an expression vector in which the nucleic acid is operably linked to a promoter. Preferably, the nucleic acid may be prepared as part of a DNA expression vector comprising a promoter and a terminator.

Preferably the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

Alternatively, a promoter may preferably be selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

The nucleic acid molecules of the invention may be made using any suitable process known in the art. Thus, the nucleic acid molecules may be made using chemical synthesis techniques. Alternatively, the nucleic acid molecules of the invention may be made using molecular biology techniques.

The nucleic acid molecules and expression vectors of the present invention may be preferably designed in silico, and then synthesised by conventional synthesis techniques, including conventional DNA synthesis techniques.

The above-mentioned nucleic acid sequence information is optionally modified for codon-biasing according to the ultimate host cell (e.g. E. coli) expression system that is to be employed.

The present invention provides a nucleotide sequence encoding an engineered clostridial neurotoxin of the present invention. The nucleotide sequence of the invention encodes a polypeptide comprising a furin cleavage site of SEQ ID NO: 1.

The nucleotide sequence may comprise a sequence having at least 70% sequence identity to SEQ ID NO: 23. The nucleotide sequence may comprise a sequence having at least 80% or 90% sequence identity to SEQ ID NO: 23. Preferably the nucleotide sequence comprises (more preferably consists of) SEQ ID NO: 23. The nucleotide sequence may encode an engineered clostridial neurotoxin having at least 70% sequence identity to one or more of SEQ ID NOs: 24 or 70 to 78. The nucleotide sequence may encode an engineered clostridial neurotoxin having at least 80% or 90% sequence identity to one or more of SEQ ID NOs: 24 or 70 to 78. Preferably the nucleotide sequence encode an engineered clostridial neurotoxin comprising (more preferably consisting of) any one of SEQ ID NOs: 24 or 70 to 78.

The terms “nucleotide sequence” and “nucleic acid” and “polynucleotide” are used synonymously herein. Preferably the nucleotide sequence is a DNA sequence.

The invention provides a method of producing a single-chain (engineered) clostridial neurotoxin protein having a light chain and a heavy chain, the method comprising expressing a polynucleotide or expression vector described herein in a suitable host cell, and recovering the expressed engineered clostridial neurotoxin. Recovering the expressed engineered clostridial neurotoxin may comprise lysing the host cell to provide a host cell homogenate containing the single-chain (engineered) clostridial neurotoxin protein, and/or isolating the single-chain (engineered) clostridial neurotoxin protein. Said method may further comprise a step of introducing the polynucleotide or expression vector described herein into the host cell. Suitable host cells include bacterial cell lines used for the recombinant production of clostridial neurotoxins, particularly Escherichia coli cells.

The present invention provides a method for proteolytically processing an (engineered) clostridial neurotoxin of the present invention into a corresponding di-chain clostridial neurotoxin, the method comprising contacting the (engineered) clostridial neurotoxin with furin thereby producing a di-chain clostridial neurotoxin (e.g. wherein the light chain and heavy chain are joined together by a disulphide bond).

The present invention therefore provides a di-chain clostridial neurotoxin obtainable by a method of the invention.

The term “obtainable” as used herein also encompasses the term “obtained”. Preferably the term “obtainable” means obtained.

### Activation of an Engineered Clostridial Neurotoxin

The invention provides a method for proteolytically processing an engineered clostridial neurotoxin of the invention into a corresponding di-chain clostridial neurotoxin, the method comprising contacting the engineered clostridial neurotoxin with furin, thereby producing a di-chain clostridial neurotoxin. Said contacting may be in vitro, ex vivo, or in vivo, preferably in vivo. As such, the therapeutic methods and uses of the invention may comprise the in vivo activation of an engineered clostridial neurotoxin of the invention by cleavage at the furin activation site by furin expression on or by target cells.

Thus, a method of the invention may further comprise contacting an engineered clostridial neurotoxin with furin thereby producing a corresponding di-chain engineered clostridial neurotoxin. Preferably said contacting occurs in vivo.

The invention also provides a method for proteolytically processing a single-chain clostridial neurotoxin into a corresponding di-chain clostridial neurotoxin, the method comprising: (a) providing a single-chain clostridial neurotoxin; and (b) contacting the single-chain clostridial neurotoxin with furin; wherein the single-chain clostridial neurotoxin has an activation loop comprising or consisting of a furin cleavage site as described herein (e.g. SEQ ID NO: 1, SEQ ID NO: 2, SEQ ID NO: 3, SEQ ID NO: 4 or SEQ ID NO: 5); and wherein furin hydrolyses a peptide bond of the activation loop thereby producing a di-chain clostridial neurotoxin. Preferably said contacting occurs in vivo.

The present invention encompasses contacting a single-chain clostridial neurotoxin (e.g. an engineered clostridial neurotoxin of the invention) with furin, wherein furin is capable of hydrolysing a peptide bond in an activation loop of the single-chain clostridial neurotoxin thereby producing a di-chain clostridial neurotoxin. Preferably said contacting occurs in vivo.

Furin is a proprotein convertase with specificity for a range of motifs, cleaving near paired arginine residues that are separated by two amino acids. The term “furin” encompasses furin described herein, as well as any protease having structural and/or functional similarity (preferably structural and functional similarity) that is capable of hydrolysing a peptide bond of SEQ ID NO: 1. A suitable furin is human furin, which has UniProt Accession No. P09958 (version 2 of the sequence, deposited 1 Apr. 1990). Human furin commercially available from Merck (#F2677 and #SRP6274). For in vitro and ex vivo uses, it is within the routine practice of one of ordinary skill in the art to determine the appropriate concentration/unit amount of furin to activate an engineered clostridial neurotoxin of the invention under standard/desired conditions.

In the context of the invention, the term “furin” encompasses a polypeptide sequence having at least 70% sequence identity to SEQ ID NO: 59. Thus, “furin” may comprise a polypeptide sequence having at least 80% or 90% sequence identity to SEQ ID NO: 59. Preferably a furin comprises (more preferably consists of) SEQ ID NO: 59.

The contacting can occur under any suitable conditions that result in the production of greater than 30%, 40%, 50% or 60% (preferably greater than 70%) of single-chain clostridial neurotoxin being proteolytically processed into the corresponding di-chain clostridial neurotoxin without, or without substantial, hydrolysis of a peptide bond outside of the activation loop of said clostridial neurotoxin. “Without substantial hydrolysis” may mean less than 5%, 4%, 3%, 2% or 1% of the clostridial neurotoxins contacted contain a peptide bond outside of the activation loop that has been hydrolysed by furin in a method of the invention.

The skilled person can select appropriate reaction times, temperatures, buffers, and molar ratios of protease to single-chain clostridial neurotoxin to achieve the above. Optimisation of such conditions can be determined empirically using routine techniques, such as SDS-PAGE (e.g. stained with Coomassie or a dye of similar sensitivity) visual analysis of the reaction products following said contacting or spectrometric techniques (e.g. mass spectrometry).

When assessed by SDS-PAGE (e.g. stained with Coomassie or a dye of similar sensitivity), a method of the invention preferably results in the production of a clostridial neurotoxin L-chain and H-chain only.

The proteolytic processing by furin in a method of the invention typically results in the production of less than 5 degradation products of a clostridial neurotoxin L-chain or H-chain, more preferably less than 4, 3, 2 or 1 degradation products. Preferably, the L-chain and H-chain produced by a method of the invention are full-length L-chain and H-chain.

Preferably, processing by furin in a method of the invention hydrolyses only the peptide bond immediately C-terminal to the C-terminal Arg of SEQ ID NO: 1.

For in vitro furin-mediated activation of engineered clostridial neurotoxins of the invention, any appropriate conditions for activation may be used. It is within the routine practice of one of ordinary skill in the art to determine suitable conditions. By way of non-limiting example, 15 Units of furin may be used per 1 mg of engineered clostridial neurotoxin, with activation carried out overnight at a temperature of about 25° C.

Many cells endogenously express furin, in particular in the Golgi apparatus and nucleoplasm. Furin expressed by cells is typically also present on the cell surface. Therefore, the step of contacting a clostridial neurotoxin with furin according to the invention may occur at or in the vicinity of the surface of a cell treated with the clostridial neurotoxin. In other words, contacting a clostridial neurotoxin with furin according to the invention may involve furin endogenously present at the cell surface. Accordingly, contacting a clostridial neurotoxin with furin according to the invention may occur in vivo following administration of the clostridial neurotoxin to an individual. When the contacting step occurs in vivo, it typically involves furin endogenously present at the surface of one or more cells present in a tissue or organ to be treated according to the invention.

The invention also provides a di-chain clostridial neurotoxin that is obtainable by a method of the invention. As activation to the di-chain form occurs by cleavage at a furin cleavage site as described herein, the resulting C- and N-terminal cleaved ends of the di-chain clostridial neurotoxin will differ in sequence compared with the corresponding (pre-engineering) clostridial neurotoxin. By way of non-limiting example, proteolytic processing of the engineered single-chain BoNT/A1 of SEQ ID NO: 24 by furin will result in a di-chain having a LC with a C-terminus ending with the sequence KKR, and a HC with an N-terminus beginning STS. In contrast, conventional trypsin cleavage of the (pre-engineering) BoNT/A will result in a di-chain having a LC with a C-terminus ending with the sequence TSK, and a HC with an N-terminus beginning ALNDLC. These di-chain clostridial neurotoxins may be used in therapy as described herein. All disclosure herein in relation to therapeutic indications and formulations in the context of engineered or single-chain clostridial neurotoxins of the invention applies equally and without reservation to di-chain clostridial neurotoxin that is obtainable by a method of the invention unless otherwise stated.

### Single-Chain Clostridial Neurotoxins

The consensus in the field of clostridial neurotoxin therapeutics is that clostridial neurotoxins must be activated to their di-chain form prior to administration to patients. Activation may be via a conventional activating protease with the necessary cleavage specificity, such as Lys-C, trypsin and/or BoNT hydrolase. In line with this established prejudice in the art, to-date, single-chain clostridial neurotoxins have not used in therapy.

The inventors have also shown that, although single-chain BoNT/A1 has lower activity in vivo than native di-chain BoNT/A1, it is still capable of eliciting a therapeutic effect (as evidence by the DAS score elicited using single-chain BoNT/A1 in the Examples herein). This is surprising, as conventional wisdom in the field is that clostridial neurotoxins need to be administered in the active di-chain form to be clinically efficacious. Based on these data, the inventors are the first to appreciate that single-chain clostridial neurotoxins, such as single-chain BoNT/A1, have therapeutic potential, without requiring activation to di-chain form prior to administration.

Accordingly, the present invention provides a clostridial neurotoxin for use in a method of preventing or treating a disease or disorder for which a therapy with a botulinum neurotoxin is indicated, wherein the clostridial neurotoxin is administered to a subject in single-chain form.

The invention also provides a pharmaceutical composition comprising a clostridial neurotoxin for use in a method of preventing or treating a disease or disorder for which a therapy with a botulinum neurotoxin is indicated, wherein the clostridial neurotoxin within the pharmaceutical composition administered to a subject is in single-chain form.

Typically the single-chain clostridial neurotoxin for use in such methods, or the pharmaceutical composition comprising said single-chain clostridial neurotoxin for use in such methods, is substantially free of a di-chain form of the clostridial neurotoxin. As used herein, the term “substantially free” may be defined as the clostridial neurotoxin or pharmaceutical composition comprising less than 400 pg di-chain clostridial neurotoxin per 100 ng single-chain clostridial neurotoxin, or less than 300 pg di-chain clostridial neurotoxin per 100 ng single-chain clostridial neurotoxin, or less than 200 pg di-chain clostridial neurotoxin per 100 ng single-chain clostridial neurotoxin, or less than 100 pg di-chain clostridial neurotoxin per 100 ng single-chain clostridial neurotoxin, or less than 50 pg di-chain clostridial neurotoxin per 100 ng single-chain clostridial neurotoxin. A pharmaceutical composition of the invention that is substantially free of di-chain clostridial neurotoxin may comprises less than 0.5%, less than 0.4%, less than 0.3%, less than 0.2%, less than 0.1% or less than 0.05% di-chain clostridial neurotoxin, preferably less than 0.1% di-chain clostridial neurotoxin.

As demonstrated in the Examples herein, single-chain clostridial neurotoxins are typically less potent than the di-chain form of the corresponding clostridial neurotoxin (as the di-chain is the active form). By way of non-limiting example, a clostridial neurotoxin in single-chain form may have potency of at least 30%, at least 40%, at least 50%, at least 60%, at least 70%, at least 80% or more compared with the corresponding di-chain clostridial neurotoxin (particularly when single-chain and di-chain forms are administered at the same dose). By way of further non-limiting example, a clostridial neurotoxin in single-chain form may be five-fold, ten-fold or 20-fold less potent compared with the corresponding di-chain clostridial neurotoxin (particularly when single-chain and di-chain forms are administered at the same dose). By way of further non-limiting example, a clostridial neurotoxin in single-chain form may have a time to reach half-maximal paralysis that is at least 25%, at least 50%, at least 60%, at least 70%, at least 80%, at least 85%, at least 90%, at least 95%, at least 96%, at least 97%, at least 98%, at least 99% longer or more compared with the corresponding di-chain clostridial neurotoxin (particularly when single-chain and di-chain forms are administered at the same dose). Potency may be measured using any appropriate assay, conventional examples of which are described herein.

Regardless of any difference in potency between single-chain and di-chain forms of the same clostridial neurotoxin, the same therapeutic effect may potentially be achieved using a single-chain clostridial neurotoxin at an increased dose, relative to the therapeutic dose of the di-chain form of the same clostridial neurotoxin. The single-chain clostridial neurotoxin may be used at a dose of at least 2 times (2×), at least 3×, at least 4×, at least 5×, at least 10×, at least 15×, at least 20× the dose of the corresponding di-chain clostridial neurotoxin.

Use of a single-chain form of a clostridial neurotoxin offers several potential advantages over the use of the corresponding di-chain clostridial neurotoxin. Administration of a clostridial neurotoxin in single-chain form give rise to fewer and/or less severe side-effects compared with administration of the corresponding di-chain clostridial neurotoxin, particularly if administered at the same dose.

Another potential advantage of using single-chain clostridial neurotoxins is that GMP production of single-chain clostridial neurotoxins to produce clinically acceptable pharmaceutical/cosmetic compositions would be easier and cheaper. This is because the protease conventionally used to cleave single-chain clostridial neurotoxins into active di-chain form during the production process and prior to administration is a significant reagent cost, and removing the need for said protease would therefore reduce manufacturing costs. In addition, conventional manufacturing protocols necessarily involve one or more additional purification steps to purify di-chain clostridial neurotoxin from the activating protease. If there is no need for activation prior to administration, and hence no need to use a protease in the manufacturing process, these additional purification steps may also be omitted, further reducing cost, complexity of the manufacturing process and production time.

The invention also provides the use of a cosmetic composition comprising a single-chain clostridial neurotoxin, and a cosmetically acceptable carrier, excipient, diluent, adjuvant, propellant and/or salt, for preventing or alleviating a cosmetic indication for which the application of a botulinum neurotoxin is indicated, wherein the single-chain clostridial neurotoxin is administered to a subject in single-chain form.

The single-chain clostridial neurotoxin for use according to the invention are typically cleaved to produce active di-chain clostridial neurotoxin by endogenous protease(s) present within the subject. Typically the target cells of the subject express endogenous protease(s) which activates the single-chain clostridial neurotoxin into di-chain form. Accordingly, activation of a single-chain clostridial neurotoxin administered to a subject according to the invention typically occurs in vivo following administration of the single-chain clostridial neurotoxin to the subject. The protease(s) is typically endogenously present in one or more cells present in a tissue or organ to be treated according to the invention.

Any and all disclosure herein in relation to clostridial neurotoxins applies equally and without reservation to aspects of the invention relating to treatment of subjects with single-chain clostridial neurotoxin, unless expressly stated to the contrary. By way of non-limiting example, the invention relates to the treatment of subjects with a single-chain clostridial neurotoxin which is a single-chain form of any BoNT serotype, particularly BoNT/A, preferably BONT/A1. Exemplary BONT sequences, particularly BoNT/A and even more particularly BONT/A1 are described herein. By way of further non-limiting example, the invention relates to the treatment of subjects with a single-chain clostridial neurotoxin which is a single-chain form of a re-targeted clostridial neurotoxins, a chimeric clostridial neurotoxin, a hybrid clostridial neurotoxin or any fragment or variant thereof as described herein.

### Therapy and Formulations

A clostridial neurotoxin of the present invention suitably finds utility in medicine and/or in cosmetics. In use, as the engineered clostridial neurotoxin of the invention may be cleaved in vivo by furin as described herein, the clostridial neurotoxin is preferably in a single-chain form for administration. Alternatively, the engineered clostridial neurotoxin of the invention may be for administration in di-chain form (e.g. having been obtained by a method of the invention).

The (engineered) clostridial neurotoxins of the invention may be used to prevent or treat certain medical or cosmetic diseases and conditions. Thus, in a further aspect, the present invention provides an (engineered) clostridial neurotoxin as described above, for use in medicine. In addition, as described herein, the invention relates to single-chain clostridial neurotoxins for use to prevent or treat certain medical or cosmetic diseases and conditions, wherein the single-chain clostridial neurotoxin is administered to a subject. In addition, as described herein, the invention relates to a di-chain clostridial neurotoxin that is obtainable by a method of the invention for use to prevent or treat certain medical or cosmetic diseases and conditions, wherein the di-chain clostridial neurotoxin that is obtainable by a method of the invention is administered to a subject. Thus, in a further aspect, the present invention provides an (engineered) clostridial neurotoxin as described above, for use in medicine.

Accordingly, the present invention provides a clostridial neurotoxin (e.g. an engineered clostridial neurotoxin) as described above, for use in the prevention or treatment of a disease or condition selected from: a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder. In some instances, the condition may be selected from phantom pain (e.g. phantom limb pain) and bladder pain syndrome. Similarly, the invention also relates to single-chain clostridial neurotoxins and di-chain clostridial neurotoxins that are obtainable by a method of the invention for use in the treatment or prevention of the above-mentioned diseases or conditions.

Where a clostridial neurotoxin of the invention (e.g. an engineered clostridial neurotoxin) comprises a BoNT/X sequence (or portion thereof) said clostridial neurotoxin may be able to target other types of secretory cells other than neurons, due to its ability to cleave VAMP4, VAMP5 and/or Ykt6. In some embodiments, the secretory cell targeted is a secretory immune cell. A “secretory immune cell” as used herein, refers to immune cells that secrets cytokines, chemokines, or antibodies. Such secretory immune cells may be innate immune cells including, without limitation, natural killer cells, mast cells, eosinophils, basophils, macrophages, neutrophils, and dendritic cells. Secretory immune cells that secret antibodies (e.g. white blood cells) may also be targeted by the clostridial neurotoxins of the present disclosure. Non-limiting examples of antibody secreting cells include, without limitation, plasma B cells, plasmocytes, plasmacytes, and effector B cells. In some embodiments, the clostridial neurotoxin may modulate an immune response. Thus, further contemplated herein are therapeutic use of a clostridial neurotoxin of the invention to treat a condition associated with unwanted secretion, preferably unwanted immune secretion. Conditions associated with unwanted immune secretion include, without limitation: inflammation, psoriasis, allergy, haemophagocytic lymphohistiocytosis, and alcoholic pancreatic disease.

The invention also provides the use of a clostridial neurotoxin (e.g. an engineered clostridial neurotoxin, a single-chain clostridial neurotoxin, or a di-chain clostridial neurotoxin that is obtainable by a method of the invention) as described above, in the manufacture of a medicament for use in a method for preventing or treating a disease or disorder as described herein.

The invention also provides a method of treating a disease or disorder as described herein, said method comprising administering a therapeutically effective amount of an clostridial neurotoxin (e.g. an engineered clostridial neurotoxin, a single-chain clostridial neurotoxin, or a di-chain clostridial neurotoxin that is obtainable by a method of the invention) as described above to a subject in need thereof.

The invention provides a pharmaceutical composition comprising an (engineered) clostridial neurotoxin or a di-chain clostridial neurotoxin of the invention and a pharmaceutically acceptable carrier, excipient, adjuvant, propellant and/or salt. Preferably the (engineered) clostridial neurotoxin is in single-chain form (e.g. engineered to comprise a furin cleavage site).

The invention also provides a cosmetic composition comprising an (engineered) clostridial neurotoxin of the invention or a di-chain clostridial neurotoxins of the invention and a cosmetically acceptable carrier, excipient, diluent, adjuvant, propellant and/or salt. The invention also provides the use of a cosmetic composition comprising a clostridial neurotoxin (e.g. an engineered clostridial neurotoxin, a single-chain clostridial neurotoxin, or a di-chain clostridial neurotoxin that is obtainable by a method of the invention) for preventing or alleviating a cosmetic indication for which the application of a botulinum neurotoxin is indicated. The invention also provides the use of a cosmetic composition comprising a clostridial neurotoxin (e.g. an engineered clostridial neurotoxin, a single-chain clostridial neurotoxin, or a di-chain clostridial neurotoxin that is obtainable by a method of the invention) for preventing or alleviating a cosmetic indication for which the application of a botulinum neurotoxin is indicated. Preferably the (engineered) clostridial neurotoxin is in single-chain form (e.g. engineered to comprise a furin cleavage site).

The clostridial neurotoxins of the present invention (e.g. an engineered clostridial neurotoxin) may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In the case of a clostridial neurotoxin (e.g. an engineered clostridial neurotoxin) that is to be delivered locally, the clostridial neurotoxin (e.g. an engineered clostridial neurotoxin) may be formulated as a cream (e.g. for topical application), or for sub-dermal injection.

Local delivery means may include an aerosol, or other spray (e.g. a nebuliser). In this regard, an aerosol formulation of a clostridial neurotoxin (e.g. an engineered clostridial neurotoxin) enables delivery to the lungs and/or other nasal and/or bronchial or airway passages.

Clostridial neurotoxins of the invention (e.g. an engineered clostridial neurotoxin) may be administered to a patient by intrathecal or epidural injection in the spinal column at the level of the spinal segment involved in the innervation of an affected organ.

A preferred route of administration is via laproscopic and/or localised, particularly intramuscular, injection.

The dosage ranges for administration of the (engineered) clostridial neurotoxins of the present invention are those to produce the desired therapeutic effect. It will be appreciated that the dosage range required depends on the precise nature of the (engineered) clostridial neurotoxin or composition, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

Suitable daily dosages (per kg weight of patient) are in the range 0.0001-1 ng/kg, preferably 0.0001-0.5 ng/kg, more preferably 0.002-0.5 ng/kg, and particularly preferably 0.004-0.5 ng/kg. The unit dosage can vary from less than 1 picogram to 30 ng, but typically will be in the region of 0.01 to 1 ng per dose, which may be administered daily or preferably less frequently, such as weekly, monthly or every six months.

A particularly preferred dosing regimen is based on 0.05 ng of (engineered) clostridial neurotoxin as the 1X dose. In this regard, preferred dosages are in the range 1X-100X (i.e. 0.05-5 ng).

Fluid dosage forms are typically prepared utilising the clostridial neurotoxin (e.g. an engineered clostridial neurotoxin) and a pyrogen-free sterile vehicle. The clostridial neurotoxin (e.g. an engineered clostridial neurotoxin), depending on the vehicle and concentration used, can be either dissolved or suspended in the vehicle. In preparing solutions the clostridial neurotoxin (e.g. an engineered clostridial neurotoxin) can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, viral delivery systems or high-pressure aerosol impingement.

Disclosure related to the various methods of the invention are intended to be applied equally to other methods, the clostridial neurotoxins, e.g. engineered clostridial neurotoxins (whether in single-chain or di-chain forms), uses or pharmaceutical compositions, as well as medical uses of single-chain clostridial neurotoxins and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Patent No. 5,223,409; Huse, WIPO Publication WO 92/06204) and region-directed mutagenesis (Derbyshire et al., Gene 46:145, 1986; Ner et al., DNA 7:127, 1988).

### Sequence Information

Where an initial Met amino acid residue or a corresponding initial codon is indicated in any of the following SEQ ID NOs, said residue/codon is optional.

SEQ ID NO: 1 (furin cleavage site consensus sequence)
Arg-Xaa-Yaa-Arg
SEQ ID NO: 2 (furin cleavage site consensus sequence)
Arg-Xaa-Lys-Arg
SEQ ID NO: 3 (furin cleavage site consensus sequence)
Arg-Xaa-Arg-Arg
SEQ ID NO: 4 (furin cleavage site)
Arg-Lys-Lys-Arg
SEQ ID NO: 5 (furin cleavage site)
KQKSSNSRKKR
SEQ ID NO: 6 (furin cleavage site)
SRKKRS
SEQ ID NO: 7 (furin cleavage site)
SRKRRS
SEQ ID NO: 8 (furin cleavage site)
SKRKKRS
SEQ ID NO: 9 (furin cleavage site)
SKRKRRS
SEQ ID NO: 10 (furin cleavage site)
TSSKSRRRKKRSTS
SEQ ID NO: 11 (furin cleavage site)
AGLITGGRRTRRSI
SEQ ID NO: 12 (furin cleavage site)
KVADSLSTRKQKR
SEQ ID NO: 13 (furin cleavage site)
LATGLRNTSQRSRRRKKRGL
SEQ ID NO: 14 (exogenous activation loop based on BoNT/A1 activation loop with furin
cleavage site)
CVRGIITSKTKSLSRKKRSALNDLC
SEQ ID NO: 15 (exogenous activation loop based on BoNT/A1 activation loop with furin
cleavage site)
CVRGIITSKTKSLSRKRRSALNDLC
SEQ ID NO: 16 (exogenous activation loop based on BoNT/A1 activation loop with furin
cleavage site)
CVRGIITSKTKSSKRKKRSALNDLC
SEQ ID NO: 17 (exogenous activation loop based on BoNT/A1 activation loop with furin
cleavage site)
CVRGIITSKTKSSKRKRRSALNDLC
SEQ ID NO: 18 (exogenous activation loop based on BoNT/A1 activation loop with furin
cleavage site)
CVRGITSSKSRRRKKRSTSALNDLC
SEQ ID NO: 19 (exogenous activation loop based on BoNT/A1 activation loop with furin
cleavage site)
CVRGIAGLITGGRRTRRSIALNDLC
SEQ ID NO: 20 (exogenous activation loop based on BoNT/A1 activation loop with furin
cleavage site)
CVRGIIKVADSLSTRKQKRALNDLC
SEQ ID NO: 21 (exogenous activation loop based on BoNT/A1 activation loop with furin
cleavage site)
CVRGILATGLRNTSQRSRRRKKRGLALNDLC
SEQ ID NO: 22 (exogenous activation loop based on BoNT/A1 activation loop with furin
cleavage site)
CVRGIKQKSSNSRKKRSTSALNDLC
SEQ ID NO: 23 (nucleic acid sequence of BoNT/A1 with a furin cleavage site)
ATGCCGTTTGTGAACAAACAGTTCAACTATAAAGATCCGGTGAACGGTGTTGATATCGC
CTATATCAAAATTCCGAATGCAGGTCAGATGCAGCCGGTTAAAGCCTTTAAAATCCATAA
CAAAATTTGGGTGATTCCGGAACGTGATACCTTTACCAATCCGGAAGAAGGTGATCTGA
ATCCGCCTCCGGAAGCAAAACAGGTTCCGGTTAGCTATTATGATAGCACCTATCTGAGC
ACCGATAACGAGAAAGATAACTATCTGAAAGGTGTGACCAAACTGTTTGAACGCATTTA
TAGTACCGATCTGGGTCGTATGCTGCTGACCAGCATTGTTCGTGGTATTCCGTTTTGGG
GTGGTAGCACCATTGATACCGAACTGAAAGTTATTGACACCAACTGCATTAATGTGATT
CAGCCGGATGGTAGCTATCGTAGCGAAGAACTGAATCTGGTTATTATTGGTCCGAGCG
CAGATATCATTCAGTTTGAATGTAAATCCTTTGGCCACGAAGTTCTGAATCTGACCCGTA
ATGGTTATGGTAGTACCCAGTATATTCGTTTCAGTCCGGATTTTACCTTTGGCTTTGAAG
AAAGCCTGGAAGTTGATACAAATCCGCTGTTAGGTGCAGGTAAATTTGCAACCGATCCG
GCAGTTACCCTGGCACATGAACTGATTCATGCCGGTCATCGTCTGTATGGTATTGCAAT
TAATCCGAACCGTGTGTTCAAAGTGAATACCAACGCATATTATGAAATGAGCGGTCTGG
AAGTGTCATTTGAAGAACTGCGTACCTTTGGTGGTCATGATGCCAAATTTATCGATAGC
CTGCAAGAAAATGAATTTCGCCTGTACTACTATAACAAATTCAAGGATATTGCGAGCACC
CTGAATAAAGCCAAAAGCATTGTTGGCACCACCGCAAGCCTGCAGTATATGAAAAATGT
GTTTAAAGAAAAATATCTGCTGAGCGAAGATACCAGCGGTAAATTTAGCGTTGACAAAC
TGAAATTCGATAAACTGTACAAGATGCTGACCGAGATTTATACCGAAGATAACTTCGTGA
AGTTTTTCAAAGTGCTGAACCGCAAAACCTACCTGAACTTTGATAAAGCCGTGTTCAAAA
TCAACATCGTGCCGAAAGTGAACTATACCATCTATGATGGTTTTAACCTGCGCAATACC
AATCTGGCAGCAAACTTTAATGGTCAGAACACCGAAATCAACAACATGAACTTTACCAAA
CTGAAGAACTTCACCGGTCTGTTCGAATTTTACAAACTGCTGTGTGTTCGTGGCATTAAA
CAAAAATCTTCGAACTCAAGAAAAAAGCGAAGTACAAGTGCCCTGAATGACCTGTGCAT
TAAAGTGAATAATTGGGACCTGTTTTTTAGCCCGAGCGAAGATAACTTTACCAACGATCT
GAATAAAGGCGAAGAAATTACCAGCGATACCAATATTGAAGCAGCCGAAGAAAACATTA
GCCTGGATCTGATTCAGCAGTATTATCTGACCTTCAACTTTGATAACGAGCCGGAAAAT
ATCAGCATTGAAAATCTGAGCAGCGATATTATTGGTCAGCTGGAACTGATGCCGAATAT
TGAACGTTTTCCGAACGGCAAAAAATACGAGCTGGATAAATACACCATGTTCCATTATCT
GCGTGCCCAAGAATTTGAACATGGTAAAAGCCGTATTGCCCTGACCAATTCAGTTAATG
AAGCACTGCTGAACCCGAGCCGTGTTTATACCTTTTTTAGCAGCGATTACGTGAAAAAG
GTGAACAAAGCAACCGAAGCAGCAATGTTTTTAGGTTGGGTTGAACAGCTGGTGTATGA
TTTCACCGATGAAACCAGCGAAGTTAGCACCACCGATAAAATTGCAGATATCACCATTA
TCATCCCGTATATTGGTCCGGCACTGAATATTGGCAATATGCTGTATAAAGATGATTTCG
TGGGTGCCCTGATTTTTAGCGGTGCAGTTATTCTGCTGGAATTTATTCCGGAAATTGCC
ATTCCGGTTCTGGGCACCTTTGCACTGGTTAGCTATATTGCAAATAAAGTTCTGACCGT
GCAGACCATTGATAATGCACTGAGCAAACGTAACGAGAAATGGGATGAAGTGTACAAAT
ATATCGTGACCAATTGGCTGGCCAAAGTTAATACCCAGATTGATCTGATCCGCAAAAAA
ATGAAAGAAGCCCTGGAAAATCAGGCAGAAGCAACCAAAGCCATTATCAACTATCAGTA
TAACCAGTACACCGAAGAAGAGAAAAACAACATCAACTTCAACATCGATGACCTGAGCA
GCAAACTGAATGAAAGCATCAATAAGGCCATGATTAACATCAACAAATTTCTGAATCAGT
GCAGCGTGAGCTATCTGATGAATAGCATGATTCCGTATGGTGTGAAACGCCTGGAAGA
TTTTGATGCAAGCCTGAAAGATGCGCTGCTGAAATATATCTATGATAATCGTGGCACCC
TGATTGGCCAGGTTGATCGTCTGAAAGATAAAGTTAACAATACCCTGAGTACCGACATT
CCGTTTCAGCTGAGCAAATATGTTGATAATCAGCGTCTGCTGAGCACCTTTACCGAATA
TATCAAGAACATCATCAACACCAGCATTCTGAATCTGCGCTATGAAAGCAATCATCTGAT
CGATCTGAGCCGTTATGCAAGCAAAATCAACATTGGTAGCAAAGTGAACTTCGACCCGA
TTGATAAAAACCAGATTCAGCTGTTTAATCTGGAAAGCAGCAAAATCGAAGTGATCCTG
AAAAACGCCATTGTGTATAACAGCATGTATGAGAATTTCTCGACCAGCTTTTGGATTCGC
ATTCCGAAATACTTTAATAGCATCAGCCTGAACAACGAGTACACCATTATTAACTGCATG
GAAAACAATAGCGGTTGGAAAGTGAGCCTGAATTATGGTGAAATTATCTGGACCCTGCA
GGATACCCAAGAAATCAAACAGCGTGTTGTGTTCAAATACAGCCAGATGATTAATATCA
GCGACTATATCAACCGCTGGATCTTTGTTACCATTACCAATAATCGCCTGAATAACAGCA
AGATCTATATTAACGGTCGCCTGATTGATCAGAAACCGATTAGCAATCTGGGCAATATT
CATGCGAGCAACAACATTATGTTTAAACTGGATGGTTGCCGTGATACCCATCGTTATATT
TGGATCAAATACTTCAACCTGTTTGATAAAGAACTGAACGAAAAAGAAATTAAAGACCTG
TACGACAACCAGAGCAATTCCGGTATTCTGAAAGACTTTTGGGGAGATTATCTGCAGTA
TGACAAACCGTATTATATGCTGAACCTGTATGACCCGAACAAATATGTGGATGTGAACA
ATGTTGGTATCCGTGGCTATATGTATCTGAAAGGTCCGCGTGGTAGCGTTATGACCACC
AACATTTATCTGAATAGCAGCCTGTATCGCGGTACGAAATTTATCATTAAAAAGTATGCC
AGCGGCAACAAGGATAATATTGTGCGTAATAATGATCGCGTGTACATTAACGTTGTGGT
GAAGAATAAAGAATATCGCCTGGCAACCAATGCAAGCCAGGCAGGCGTTGAAAAAATT
CTGAGCGCACTGGAAATTCCGGATGTTGGTAATCTGAGCCAGGTTGTTGTTATGAAAAG
CAAAAATGATCAGGGCATCACCAACAAGTGCAAAATGAATCTGCAGGACAATAACGGCA
ACGATATTGGTTTTATTGGCTTCCACCAGTTCAACAATATTGCGAAACTGGTTGCAAGCA
ATTGGTATAATCGTCAGATTGAACGTAGCAGTCGTACCCTGGGTTGTAGCTGGGAATTT
ATCCCTGTGGATGATGGTTGGGGTGAACGTCCGCTG
SEQ ID NO: 24 (amino acid sequence of BoNT/A1 with a furin cleavage site)
MPFVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGSTIDT
ELKVIDTNCINVIQPDGSYRSEELNLVIIGPSADIIQFECKSFGHEVLNLTRNGYGSTQYIRFSP
DFTFGFEESLEVDTNPLLGAGKFATDPAVTLAHELIHAGHRLYGIAINPNRVFKVNTNAYYE
MSGLEVSFEELRTFGGHDAKFIDSLQENEFRLYYYNKFKDIASTLNKAKSIVGTTASLQYMK
NVFKEKYLLSEDTSGKFSVDKLKFDKLYKMLTEIYTEDNFVKFFKVLNRKTYLNFDKAVFKINI
VPKVNYTIYDGFNLRNTNLAANFNGQNTEINNMNFTKLKNFTGLFEFYKLLCVRGIKQKSSN
SRKKRSTSALNDLCIKVNNWDLFFSPSEDNFTNDLNKGEEITSDTNIEAAEENISLDLIQQYY
LTFNFDNEPENISIENLSSDIIGQLELMPNIERFPNGKKYELDKYTMFHYLRAQEFEHGKSRIA
LTNSVNEALLNPSRVYTFFSSDYVKKVNKATEAAMFLGWVEQLVYDFTDETSEVSTTDKIAD
ITIIIPYIGPALNIGNMLYKDDFVGALIFSGAVILLEFIPEIAIPVLGTFALVSYIANKVLTVQTIDNA
LSKRNEKWDEVYKYIVTNWLAKVNTQIDLIRKKMKEALENQAEATKAIINYQYNQYTEEEKN
NINFNIDDLSSKLNESINKAMININKFLNQCSVSYLMNSMIPYGVKRLEDFDASLKDALLKYIY
DNRGTLIGQVDRLKDKVNNTLSTDIPFQLSKYVDNQRLLSTFTEYIKNIINTSILNLRYESNHLI
DLSRYASKINIGSKVNFDPIDKNQIQLFNLESSKIEVILKNAIVYNSMYENFSTSFWIRIPKYFN
SISLNNEYTIINCMENNSGWKVSLNYGEIIWTLQDTQEIKQRVVFKYSQMINISDYINRWIFVTI
TNNRLNNSKIYINGRLIDQKPISNLGNIHASNNIMFKLDGCRDTHRYIWIKYFNLFDKELNEKEI
KDLYDNQSNSGILKDFWGDYLQYDKPYYMLNLYDPNKYVDVNNVGIRGYMYLKGPRGSVM
TTNIYLNSSLYRGTKFIIKKYASGNKDNIVRNNDRVYINVVVKNKEYRLATNASQAGVEKILSA
LEIPDVGNLSQVVVMKSKNDQGITNKCKMNLQDNNGNDIGFIGFHQFNNIAKLVASNWYNR
QIERSSRTLGCSWEFIPVDDGWGERPL
The exogenous furin cleavage site (underlined) replaces part of the endogenous BoNT/A1
activation loop (bold).
SEQ ID NO: 25 (BONT/A-UniProt P10845)
MPFVNKQFNYKDPVNGVDIAYIKIPNVGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGENLRNTNLAANENGQNTEINNMNFTKLKNFT
GLFEFYKLLCVRGIITSKTKSLDKGYNKALNDLCIKVNNWDLFFSPSEDNETNDLNKGEE
ITSDTNIEAAEENISLDLIQQYYLTENEDNEPENISIENLSSDIIGQLELMPNIEREPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINENIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIINTSILNLRYESNHLIDLSRYASKINI
GSKVNFDPIDKNQIQLENLESSKIEVILKNAIVYNSMYENFSTSFWIRIPKYENSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYENLEDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
In some embodiments, valine27 may be substituted with alanine, as shown in SEQ ID NO: 68
SEQ ID NO: 26 (BoNT/B-UniProt P10844)
MPVTINNENYNDPIDNNNIIMMEPPFARGTGRYYKAFKITDRIWIIPERYTFGYKPEDEN
KSSGIFNRDVCEYYDPDYLNTNDKKNIFLQTMIKLENRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVENNVQENKGASIFNRRGYFSDPALILMHELIHVLHGLY
GIKVDDLPIVPNEKKFFMQSTDAIQAEELYTFGGQDPSIITPSTDKSIYDKVLQNERGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGFNISDKDMEKEYRGQNKAINKQA
YEEISKEHLAVYKIQMCKSVKAPGICIDVDNEDLFFIADKNSESDDLSKNERIEYNTQSN
YIENDFPINELILDTDLISKIELPSENTESLTDENVDVPVYEKQPAIKKIFTDENTIFQY
LYSQTFPLDIRDISLTSSEDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDENDINSKLNEGINQAIDNINNFINGCSV
SYLMKKMIPLAVEKLLDFDNTLKKNLLNYIDENKLYLIGSAEYEKSKVNKYLKTIMPEDL
SIYTNDTILIEMENKYNSEILNNIILNLRYKDNNLIDLSGYGAKVEVYDGVELNDKNQFK
LTSSANSKIRVTQNQNIIFNSVELDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFENLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
SEQ ID NO: 27 (BoNT/C-UniProt P18640)
MPITINNFNYSDPVDNKNILYLDTHLNTLANEPEKAFRITGNIWVIPDRFSRNSNPNLNK
PPRVTSPKSGYYDPNYLSTDSDKDPFLKEIIKLFKRINSREIGEELIYRLSTDIPFPGNN
NTPINTFDFDVDENSVDVKTRQGNNWVKTGSINPSVIITGPRENIIDPETSTEKLINNTE
AAQEGFGALSIISISPREMLTYSNATNDVGEGRFSKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSENKYIGEYKQKLIRKYRFVVESSGEVTVNRNKFVELYNELTQIFTE
FNYAKIYNVQNRKIYLSNVYTPVTANILDDNVYDIQNGENIPKSNLNVLFMGQNLSRNPA
LRKVNPENMLYLFTKFCHKAIDGRSLYNKTLDCRELLVKNTDLPFIGDISDVKTDIFLRK
DINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEFDRNTKAKLINLIDSHNIILVGEVDKLKAKVNNSF
QNTIPFNIFSYTNNSLLKDIINEYENNINDSKILSLQNRKNTLVDTSGYNAEVSEEGDVQ
LNPIFPFDEKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKEL
DGKDINILFNSLQYTNVVKDYWGNDLRYNKEYYMVNIDYLNRYMYANSRQIVENTRRNNN
DFNEGYKIIIKRIRGNTNDTRVRGGDILYFDMTINNKAYNLFMKNETMYADNHSTEDIYA
IGLREQTKDINDNIIFQIQPMNNTYYYASQIFKSNENGENISGICSIGTYRFRLGGDWYR
HNYLVPTVKQGNYASLLESTSTHWGFVPVSE
SEQ ID NO: 28 (BoNT/D-UniProt P19321)
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPFMGDS
STPEDTFDFTRHTTNIAVEKFENGSWKVTNIITPSVLIFGPLPNILDYTASLTLQGQQSN
PSFEGFGTLSILKVAPEFLLTFSDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNEDKDNTGNFVVNIDKENSLYSDLTNVMSE
VVYSSQYNVKNRTHYFSRHYLPVFANILDDNIYTIRDGENLINKGENIENSGQNIERNPA
LQKLSSESVVDLFTKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLFLNWANE
VVEDFTTNIMKKDTLDKISDVSVIIPYIGPALNIGNSALRGNENQAFATAGVAFLLEGFP
EFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIR
ECSVTYLFKNMLPKVIDELNKEDLRTKTELINLIDSHNIILVGEVDRLKAKVNESFENTM
PFNIFSYTNNSLLKDIINEYENSINDSKILSLQNKKNALVDTSGYNAEVRVGDNVQLNTI
YTNDFKLSSSGDKIIVNLNNNILYSAIYENSSVSFWIKISKDLINSHNEYTIINSIEQNS
GWKLCIRNGNIEWILQDVNRKYKSLIFDYSESLSHTGYTNKWFFVTITNNIMGYMKLYIN
GELKQSQKIEDLDEVKLDKTIVEGIDENIDENQMLWIRDENIFSKELSNEDINIVYEGQI
LRNVIKDYWGNPLKFDTEYYIINDNYIDRYIAPESNVLVLVQYPDRSKLYTGNPITIKSV
SDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRFSFKNAYTPVAVTNYETKLLS
TSSFWKFISRDPGWVE
SEQ ID NO: 29 (BONT/E-UniProt Q00496)
MPKINSFNYNDPVNDRTILYIKPGGCQEFYKSENIMKNIWIIPERNVIGTTPQDFHPPTS
LKNGDSSYYDPNYLQSDEEKDRFLKIVTKIFNRINNNLSGGILLEELSKANPYLGNDNTP
DNQFHIGDASAVEIKFSNGSQDILLPNVIIMGAEPDLFETNSSNISLRNNYMPSNHRFGS
IAIVTESPEYSFRENDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLRTKFQVKCRQTYIGQYKYFKL
SNLLNDSIYNISEGYNINNLKVNERGQNANLNPRIITPITGRGLVKKIIRFCKNIVSVKG
IRKSICIEINNGELFFVASENSYNDDNINTPKEIDDTVTSNNNYENDLDQVILNENSESA
PGLSDEKLNLTIQNDAYIPKYDSNGTSDIEQHDVNELNVEFYLDAQKVPEGENNVNLTSS
IDTALLEQPKIYTFFSSEFINNVNKPVQAALFVSWIQQVLVDETTEANQKSTVDKIADIS
IVVPYIGLALNIGNEAQKGNEKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRELTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SQNDYIIYDNKYKNESISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYFNIFDKELDETEIQTLYSNEPNTNILKDFWGNYL
LYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRENQVVVMNSVGNCTMNF
KNNNGNNIGLLGFKADTVVASTWYYTHMRDHTNSNGCFWNFISEEHGWQEK
SEQ ID NO: 30 (BONT/F-UniProt A7GBG3)
MPVVINSFNYNDPVNDDTILYMQIPYEEKSKKYYKAFEIMRNVWIIPERNTIGTDPSDED
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTENDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKF
KVKCRNTYFIKYGFLKVPNLLDDDIYTVSEGFNIGNLAVNNRGQNIKLNPKIIDSIPDKG
LVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDC
IRNNNSGWKISLNYNKIIWTLQDTAGNNQKLVENYTQMISISDYINKWIFVTITNNRLGN
SRIYINGNLIDEKSISNLGDIHVSDNILFKIVGCNDTRYVGIRYFKVEDTELGKTEIETL
YSDEPDPSILKDFWGNYLLYNKRYYLLNLLRTDKSITQNSNFLNINQQRGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
SEQ ID NO: 31 (BoNT/G-UniProt Q60393)
MPVNIKXFNYNDPINNDDIIMMEPENDPGPGTYYKAFRIIDRIWIVPERFTYGFQPDQEN
ASTGVFSKDVYEYYDPTYLKTDAEKDKFLKTMIKLFNRINSKPSGQRLLDMIVDAIPYLG
NASTPPDKFAANVANVSINKKIIQPGAEDQIKGLMTNLIIFGPGPVLSDNFTDSMIMNGH
SPISEGFGARMMIRFCPSCLNVENNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKEDKLYKALMFGFTETNL
AGEYGIKTRYSYFSEYLPPIKTEKLLDNTIYTQNEGENIASKNLKTEENGQNKAVNKEAY
EEISLEHLVIYRIAMCKPVMYKNTGKSEQCIIVNNEDLFFIANKDSFSKDLAKAETIAYN
TQNNTIENNFSIDQLILDNDLSSGIDLPNENTEPFTNEDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENEKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDENDIDFKLNQSINLAINNIDDFI
NQCSISYLMNRMIPLAVKKLKDFDDNLKRDLLEYIDTNELYLLDEVNILKSKVNRHLKDS
IPFDLSLYTKDTILIQVENNYISNISSNAILSLSYRGGRLIDSSGYGATMNVGSDVIEND
IGNGQFKLNNSENSNITAHQSKFVVYDSMFDNESINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDEKLINCTDTTKFVWIKDENIFGRELNATEVS
SLYWIQSSTNTLKDFWGNPLRYDTQYYLENQGMQNIYIKYFSKASMGETAPRTNENNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
SEQ ID NO: 32 (Polypeptide Sequence of BoNT/X)
MKLEINKFNYNDPIDGINVITMRPPRHSDKINKGKGPFKAFQVIKNIWIVPERYNFTNNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNEDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGENISSQGSNDFQGQLLESSYFEKIESNALRAFI
KICPRNGLLYNAIYRNSKNYLNNIDLEDKKTTSKTNVSYPCSLLNGCIEVENKDLFLISN
KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDESDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKEMIKLSNSYLTKEMIPKVQDNLKNEDLETKKTLDK
FIKEKEDILGTNLSSSLRRKVSIRLNKNIAFDINDIPESEFDDLINQYKNEIEDYEVLNL
GAEDGKIKDLSGTTSDINIGSDIELADGRENKAIKIKGSENSTIKIAMNKYLRFSATDNF
SISFWIKHPKPTNLLNNGIEYTLVENENQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYFNSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKTNWYFIPKDEGWDED
SEQ ID NO: 33 (TeNT-UniProt P04958)
MPITINNFRYSDPVNNDTIIMMEPPYCKGLDIYYKAFKITDRIWIVPERYEFGTKPEDEN
PPSSLIEGASEYYDPNYLRTDSDKDRFLQTMVKLFNRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGENIESKDLKSEYKGQNMRVNT
NAFRNVDGSGLVSKLIGLCKKIIPPTNIRENLYNRTASLTDLGGELCIKIKNEDLTFIAE
KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLQWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKQLLEFDTQSKNILMQYIKANSKFIG
ITELKKLESKINKVESTPIPFSYSKNLDCWVDNEEDIDVILKKSTILNLDINNDIISDIS
GENSSVITYPDAQLVPGINGKAIHLVNNESSEVIVHKAMDIEYNDMENNFTVSFWLRVPK
VSASHLEQYGTNEYSIISSMKKHSLSIGSGWSVSLKGNNLIWTLKDSAGEVRQITFRDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWTND
SEQ ID NO: 34 (BONT/D Activation Loop)
CLRLTKNSRDDSTC
SEQ ID NO: 35 (BoNT/DC Activation Loop)
CLRLTRNSRDDSTC
SEQ ID NO: 36 (BoNT/C1 and CD Activation Loop)
CHKAIDGRSLYNKTLDC
SEQ ID NO: 37 (BoNT/A4 Activation Loop)
CVRGIITSKTKSLDEGYNKALNELC
SEQ ID NO: 38 (BoNT/A5 and A7 Activation Loop)
CVRGIITSKTKSLDEGYNKALNDLC
SEQ ID NO: 39 (BoNT/A1 and A6 Activation Loop)
CVRGIITSKTKSLDKGYNKALNDLC
SEQ ID NO: 40 (BoNT/A3 Activation Loop)
CVRGIIPFKTKSLDEGYNKALNYLC
SEQ ID NO: 41 (BoNT/A2 and A8 Activation Loop)
CVRGIIPFKTKSLDEGYNKALNDLC
SEQ ID NO: 42 (BoNT/H Activation Loop)
CSNSNTKNSLC
SEQ ID NO: 43 (BoNT/E1 to E5, E9 and E12 Activation Loop)
CKNIVSVKGIRKSIC
SEQ ID NO: 44 (BoNT/E11 Activation Loop)
CTNIFSPKGIRKSIC
SEQ ID NO: 45 (BoNT/E7, E8 and E10 Activation Loop)
CKNIVFSKGITKSIC
SEQ ID NO: 46 (BoNT/E6 Activation Loop)
CKNIVFSKGIRKSIC
SEQ ID NO: 47 (BoNT/F7 Activation Loop)
CKSIVSKKGTKNSLC
SEQ ID NO: 48 (BONT/F5 Activation Loop)
CLNSSFKKNTKKPLC
SEQ ID NO: 49 (BoNT/F1 and F6 Activation Loop)
CKSVIPRKGTKAPPRLC
SEQ ID NO: 50 (BONT/F4 Activation Loop)
CKSIIPRKGTKAPPRLC
SEQ ID NO: 51 (BoNT/F2 and F3 Activation Loop)
CKSIIPRKGTKQSPSLC
SEQ ID NO: 52 (TeNT Activation Loop)
CKKIIPPTNIRENLYNRTASLTDLGGELC
SEQ ID NO: 53 (BoNT/G Activation Loop)
CKPVMYKNTGKSEQC
SEQ ID NO: 54 (BoNT/B4 Activation Loop)
CKSVKVPGIC
SEQ ID NO: 55 (BoNT/B2, B3, B6 and B8 Activation Loop)
CKSVRAPGIC
SEQ ID NO: 56 (BoNT/B1, B5 and B7 Activation Loop)
CKSVKAPGIC
SEQ ID NO: 57 (BoNT/X Activation Loop)
CPRNGLLYNAIYRNSKNYLNNIDLEDKKTTSKTNVSYPCSLLNGC
SEQ ID NO: 58 metal coordinating SNARE cleavage motif
HEXXH
SEQ ID NO: 59 (amino acid sequence of human furin)
MELRPWLLWVVAATGTLVLLAADAQGQKVFTNTWAVRIPGGPAVANSVARKHGFLNLGQI
FGDYYHFWHRGVTKRSLSPHRPRHSRLQREPQVQWLEQQVAKRRTKRDVYQEPTDPKFP
QQWYLSGVTQRDLNVKAAWAQGYTGHGIVVSILDDGIEKNHPDLAGNYDPGASFDVNDQD
PDPQPRYTQMNDNRHGTRCAGEVAAVANNGVCGVGVAYNARIGGVRMLDGEVTDAVEA
RSLGLNPNHIHIYSASWGPEDDGKTVDGPARLAEEAFFRGVSQGRGGLGSIFVWASGNGG
REHDSCNCDGYTNSIYTLSISSATQFGNVPWYSEACSSTLATTYSSGNQNEKQIVTTDLRQ
KCTESHTGTSASAPLAAGIIALTLEANKNLTWRDMQHLVVQTSKPAHLNANDWATNGVGRK
VSHSYGYGLLDAGAMVALAQNWTTVAPQRKCIIDILTEPKDIGKRLEVRKTVTACLGEPNHIT
RLEHAQARLTLSYNRRGDLAIHLVSPMGTRSTLLAARPHDYSADGFNDWAFMTTHSWDED
PSGEWVLEIENTSEANNYGTLTKFTLVLYGTAPEGLPVPPESSGCKTLTSSQACVVCEEGF
SLHQKSCVQHCPPGFAPQVLDTHYSTENDVETIRASVCAPCHASCATCQGPALTDCLSCP
SHASLDPVEQTCSRQSQSSRESPPQQQPPRLPPEVEAGQRLRAGLLPSHLPEVVAGLSCA
FIVLVFVTVFLVLQLRSGFSFRGVKVYTMDRGLISYKGLPPEAWQEECPSDSEEDEGRGER
TAFIKDQSAL
SEQ ID NO: 60 (additional protease cleavage site)
xDxxxLL
x is any amino acid
SEQ ID NO: 61 (additional protease cleavage site)
xExxxLL
x is any amino acid
SEQ ID NO: 62 (additional protease cleavage site)
xExxxIL
x is any amino acid
SEQ ID NO: 63 (additional protease cleavage site)
xExxxLM
x is any amino acid
SEQ ID NO: 64 (additional protease cleavage site)
Y-x-x-Hy
x is any amino acid; Hy is a hydrophobic amino acid
SEQ ID NO: 65 (TEV cleavage site)
ENLYFQG
SEQ ID NO: 66 (Thrombin cleavage site)
LVPRGS
SEQ ID NO: 67 (PreScission cleavage site)
LEVLFQGP
SEQ ID NO: 68 (BoNT/A)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRESPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGENLRNTNLAANENGQNTEINNMNFTKLKNET
GLFEFYKLLCVRGIITSKTKSLDKGYNKALNDLCIKVNNWDLFFSPSEDNETNDLNKGEE
ITSDTNIEAAEENISLDLIQQYYLTENEDNEPENISIENLSSDIIGQLELMPNIEREPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDEVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINENIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIINTSILNLRYESNHLIDLSRYASKINI
GSKVNFDPIDKNQIQLENLESSKIEVILKNAIVYNSMYENESTSFWIRIPKYENSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYENLEDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
SEQ ID NO: 69: non-engineered BoNT/AB chimera
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
SytII-binding mutations E1191M and S1199Y are bold and underlined.
The endogenous activation loop is dash-underlined.
SEQ ID NO: 70: engineered BoNT/AB chimera
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIKQKSSNSRKKRSTSAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVELDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
SytII-binding mutations E1191M and S1199Y are bold and underlined.
The exogenous furin cleavage site (underlined) replaces part of the endogenous BoNT/A1
activation loop (bold).
SEQ ID NO: 71 engineered BoNT/B derived from SEQ ID NO: 26
MPVTINNENYNDPIDNNNIIMMEPPFARGTGRYYKAFKITDRIWIIPERYTFGYKPEDENKSSGIFNR
DVCEYYDPDYLNTNDKKNIFLQTMIKLENRIKSKPLGEKLLEMIINGIPYLGDRRVPLEEFNTNIASV
TVNKLISNPGEVERKKGIFANLIIFGPGPVLNENETIDIGIQNHFASREGEGGIMQMKFCPEYVSVEN
NVQENKGASIFNRRGYFSDPALILMHELIHVLHGLYGIKVDDLPIVPNEKKFFMQSTDAIQAEELYTE
GGQDPSIITPSTDKSIYDKVLQNERGIVDRLNKVLVCISDPNININIYKNKFKDKYKFVEDSEGKYSI
DVESFDKLYKSLMFGFTETNIAENYKIKTRASYFSDSLPPVKIKNLLDNEIYTIEEGENISDKDMEKE
YRGQNKAINKQAYEEISKEHLAVYKIQMCKSVKQKSSNSRKKRSTSGICIDVDNEDLFFIADKNSES
DDLSKNERIEYNTQSNYIENDEPINELILDTDLISKIELPSENTESLTDFNVDVPVYEKQPAIKKIFT
DENTIFQYLYSQTFPLDIRDISLTSSEDDALLESNKVYSFFSMDYIKTANKVVEAGLFAGWVKQIVND
FVIEANKSNTMDKIADISLIVPYIGLALNVGNETAKGNFENAFEIAGASILLEFIPELLIPVVGAFLL
ESYIDNKNKIIKTIDNALTKRNEKWSDMYGLIVAQWLSTVNTQFYTIKEGMYKALNYQAQALEEIIKY
RYNIYSEKEKSNINIDENDINSKLNEGINQAIDNINNFINGCSVSYLMKKMIPLAVEKLLDEDNTLKK
NLLNYIDENKLYLIGSAEYEKSKVNKYLKTIMPFDLSIYINDTILIEMENKYNSEILNNIILNLRYKD
NNLIDLSGYGAKVEVYDGVELNDKNQFKLTSSANSKIRVTQNQNIIENSVELDESVSFWIRIPKYKND
GIQNYIHNEYTIINCMKNNSGWKISIRGNRIIWTLIDINGKTKSVFFEYNIREDISEYINRWFFVTIT
NNLNNAKIYINGKLESNTDIKDIREVIANGEIIFKLDGDIDRTQFIWMKYFSIFNTELSQSNIEERYK
IQSYSEYLKDFWGNPLMYNKEYYMFNAGNKNSYIKLKKDSPVGEILTRSKYNQNSKYINYRDLYIGEK
FIIRRKSNSQSINDDIVRKEDYIYLDFFNLNQEWRVYTYKYFKKEEEKLFLAPISDSDEFYNTIQIKE
YDEQPTYSCQLLFKKDEESTDEIGLIGIHRFYESGIVFEEYKDYFCISKWYLKEVKRKPYNLKLGCNW
QFIPKDEGWTE
The exogenous furin cleavage site (underlined) replaces part of the endogenous BoNT/B
activation loop (bold).
SEQ ID NO: 72 engineered BoNT/C derived from SEQ ID NO: 27
MPITINNFNYSDPVDNKNILYLDTHLNTLANEPEKAFRITGNIWVIPDRFSRNSNPNLNKPPRVTSPK
SGYYDPNYLSTDSDKDPFLKEIIKLFKRINSREIGEELIYRLSTDIPFPGNNNTPINTEDEDVDENSV
DVKTRQGNNWVKTGSINPSVIITGPRENIIDPETSTFKLTNNTFAAQEGFGALSIISISPREMLTYSN
ATNDVGEGRFSKSEFCMDPILILMHELNHAMHNLYGIAIPNDQTISSVTSNIFYSQYNVKLEYAEIYA
FGGPTIDLIPKSARKYFEEKALDYYRSIAKRLNSITTANPSSENKYIGEYKQKLIRKYRFVVESSGEV
TVNRNKEVELYNELTQIFTEFNYAKIYNVQNRKIYLSNVYTPVTANILDDNVYDIQNGFNIPKSNLNV
LFMGQNLSRNPALRKVNPENMLYLFTKFCHKAIKQKSSNSRKKRSTSTLDCRELLVKNTDLPFIGDI
SDVKTDIFLRKDINEETEVIYYPDNVSVDQVILSKNTSEHGQLDLLYPSIDSESEILPGENQVFYDNR
TQNVDYLNSYYYLESQKLSDNVEDFTFTRSIEEALDNSAKVYTYFPTLANKVNAGVQGGLELMWANDV
VEDFTTNILRKDTLDKISDVSAIIPYIGPALNISNSVRRGNFTEAFAVTGVTILLEAFPEFTIPALGA
FVIYSKVQERNEIIKTIDNCLEQRIKRWKDSYEWMMGTWLSRIITQFNNISYQMYDSLNYQAGAIKAK
IDLEYKKYSGSDKENIKSQVENLKNSLDVKISEAMNNINKFIRECSVTYLFKNMLPKVIDELNEEDRN
TKAKLINLIDSHNIILVGEVDKLKAKVNNSFQNTIPFNIFSYTNNSLLKDIINEYENNINDSKILSLQ
NRKNTLVDTSGYNAEVSEEGDVQLNPIFPEDEKLGSSGEDRGKVIVTQNENIVYNSMYESFSISEWIR
INKWVSNLPGYTIIDSVKNNSGWSIGIISNELVETLKQNEDSEQSINFSYDISNNAPGYNKWFFVTVT
NNMMGNMKIYINGKLIDTIKVKELTGINFSKTITFEINKIPDTGLITSDSDNINMWIRDFYIFAKELD
GKDINILENSLQYTNVVKDYWGNDLRYNKEYYMVNIDYLNRYMYANSRQIVENTRRNNNDENEGYKII
IKRIRGNTNDTRVRGGDILYFDMTINNKAYNLFMKNETMYADNHSTEDIYAIGLREQTKDINDNIIFQ
IQPMNNTYYYASQIFKSNENGENISGICSIGTYRFRLGGDWYRHNYLVPTVKQGNYASLLESTSTHWG
FVPVSE
The exogenous furin cleavage site (underlined) replaces part of the endogenous BoNT/C
activation loop (bold).
SEQ ID NO: 73 engineered BoNT/D derived from SEQ ID NO: 28
MTWPVKDENYSDPVNDNDILYLRIPQNKLITTPVKAFMITQNIWVIPERFSSDTNPSLSKPPRPTSKY
QSYYDPSYLSTDEQKDTFLKGIIKLFKRINERDIGKKLINYLVVGSPFMGDSSTPEDTFDFTRHTTNI
AVEKFENGSWKVTNIITPSVLIFGPLPNILDYTASLTLQGQQSNPSFEGFGTLSILKVAPEFLLTESD
VTSNQSSAVLGKSIFCMDPVIALMHELTHSLHQLYGINIPSDKRIRPQVSEGFFSQDGPNVQFEELYT
FGGLDVEIIPQIERSQLREKALGHYKDIAKRLNNINKTIPSSWISNIDKYKKIFSEKYNFDKDNTGNF
VVNIDKFNSLYSDLTNVMSEVVYSSQYNVKNRTHYFSRHYLPVFANILDDNIYTIRDGENLINKGENI
ENSGQNIERNPALQKLSSESVVDLFTKVCLRLKQKSSNSRKKRSTSDSTCIKVKNNRLPYVADKDSI
SQEIFENKIITDETNVQNYSDKFSLDESILDGQVPINPEIVDPLLPNVNMEPLNLPGEEIVFYDDITK
YVDYLNSYYYLESQKLSNNVENITLTTSVEEALGYSNKIYTELPSLAEKVNKGVQAGLELNWANEVVE
DFTTNIMKKDTLDKISDVSVIIPYIGPALNIGNSALRGNENQAFATAGVAFLLEGFPEFTIPALGVET
FYSSIQEREKIIKTIENCLEQRVKRWKDSYQWMVSNWLSRITTQFNHINYQMYDSLSYQADAIKAKID
LEYKKYSGSDKENIKSQVENLKNSLDVKISEAMNNINKFIRECSVTYLFKNMLPKVIDELNKFDLRTK
TELINLIDSHNIILVGEVDRLKAKVNESFENTMPFNIFSYTNNSLLKDIINEYENSINDSKILSLQNK
KNALVDTSGYNAEVRVGDNVQLNTIYTNDEKLSSSGDKIIVNLNNNILYSAIYENSSVSFWIKISKDL
TNSHNEYTIINSIEQNSGWKLCIRNGNIEWILQDVNRKYKSLIFDYSESLSHTGYTNKWFFVTITNNI
MGYMKLYINGELKQSQKIEDLDEVKLDKTIVEGIDENIDENQMLWIRDENIFSKELSNEDINIVYEGQ
ILRNVIKDYWGNPLKEDTEYYIINDNYIDRYIAPESNVLVLVQYPDRSKLYTGNPITIKSVSDKNPYS
RILNGDNIILHMLYNSRKYMIIRDTDTIYATQGGECSQNCVYALKLQSNLGNYGIGIFSIKNIVSKNK
YCSQIFSSFRENTMLLADIYKPWRFSFKNAYTPVAVTNYETKLLSTSSFWKFISRDPGWVE
The exogenous furin cleavage site (underlined) replaces part of the endogenous BoNT/D
activation loop (bold).
SEQ ID NO: 74 engineered BoNT/E derived from SEQ ID NO: 29
MPKINSFNYNDPVNDRTILYIKPGGCQEFYKSENIMKNIWIIPERNVIGTTPQDFHPPTSLKNGDSSY
YDPNYLQSDEEKDRELKIVTKIFNRINNNLSGGILLEELSKANPYLGNDNTPDNQFHIGDASAVEIKE
SNGSQDILLPNVIIMGAEPDLFETNSSNISLRNNYMPSNHRFGSIAIVTFSPEYSFRENDNCMNEFIQ
DPALTLMHELIHSLHGLYGAKGITTKYTITQKQNPLITNIRGTNIEEFLTFGGTDLNIITSAQSNDIY
TNLLADYKKIASKLSKVQVSNPLLNPYKDVFEAKYGLDKDASGIYSVNINKENDIFKKLYSFTEFDLR
TKFQVKCRQTYIGQYKYFKLSNLLNDSIYNISEGYNINNLKVNFRGQNANLNPRIITPITGRGLVKKI
IRFCKNIKQKSSNSRKKRSTSSICIEINNGELFFVASENSYNDDNINTPKEIDDTVTSNNNYENDLD
QVILNENSESAPGLSDEKLNLTIQNDAYIPKYDSNGTSDIEQHDVNELNVFFYLDAQKVPEGENNVNL
TSSIDTALLEQPKIYTFFSSEFINNVNKPVQAALFVSWIQQVLVDETTEANQKSTVDKIADISIVVPY
IGLALNIGNEAQKGNEKDALELLGAGILLEFEPELLIPTILVFTIKSFLGSSDNKNKVIKAINNALKE
RDEKWKEVYSFIVSNWMTKINTQFNKRKEQMYQALQNQVNAIKTIIESKYNSYTLEEKNELTNKYDIK
QIENELNQKVSIAMNNIDRELTESSISYLMKIINEVKINKLREYDENVKTYLLNYIIQHGSILGESQQ
ELNSMVTDTLNNSIPFKLSSYTDDKILISYENKFFKRIKSSSVLNMRYKNDKYVDTSGYDSNININGD
VYKYPTNKNQFGIYNDKLSEVNISQNDYIIYDNKYKNFSISFWVRIPNYDNKIVNVNNEYTIINCMRD
NNSGWKVSLNHNEIIWTFEDNRGINQKLAFNYGNANGISDYINKWIFVTITNDRLGDSKLYINGNLID
QKSILNLGNIHVSDNILFKIVNCSYTRYIGIRYFNIFDKELDETEIQTLYSNEPNTNILKDEWGNYL
LYDKEYYLLNVLKPNNFIDRRKDSTLSINNIRSTILLANRLYSGIKVKIQRVNNSSTNDNLVRKNDQV
YINFVASKTHLFPLYADTATTNKEKTIKISSSGNRFNQVVVMNSVGNCTMNFKNNNGNNIGLLGEKAD
TVVASTWYYTHMRDHTNSNGCFWNFISEEHGWQEK
The exogenous furin cleavage site (underlined) replaces part of the endogenous BoNT/E
activation loop (bold).
SEQ ID NO: 75 engineered BoNT/F derived from SEQ ID NO: 30
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHELIHALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGENIGNLAVNNRGQNIKLNPKIIDSIPDKGLVEKIVKFCKSVIKQKSSNSRKKRSTSR
LCIRVNNRELFFVASESSYNENDINTPKEIDDTTNLNNNYRNNLDEVILDYNSETIPQISNQTLNTLVQDDSYVP
RYDSNGTSEIEEHNVVDLNVFFYLHAQKVPEGETNISLTSSIDTALSEESQVYTFFSSEFINTINKPVHAALFIS
WINQVIRDFTTEATQKSTEDKIADISLVVPYVGLALNIGNEVQKENFKEAFELLGAGILLEFVPELLIPTILVFT
IKSFIGSSENKNKIIKAINNSLMERETKWKEIYSWIVSNWLTRINTQFNKRKEQMYQALQNQVDAIKTVIEYKYN
NYTSDERNRLESEYNINNIREELNKKVSLAMENIERFITESSIFYLMKLINEAKVSKLREYDEGVKEYLLDYISE
HRSILGNSVQELNDLVTSTLNNSIPFELSSYTNDKILILYFNKLYKKIKDNSILDMRYENNKFIDISGYGSNISI
NGDVYIYSTNRNQFGIYSSKPSEVNIAQNNDIIYNGRYQNFSISFWVRIPKYFNKVNLNNEYTIIDCIRNNNSGW
KISLNYNKIIWTLQDTAGNNQKLVENYTQMISISDYINKWIFVTITNNRLGNSRIYINGNLIDEKSISNLGDIHV
SDNILFKIVGCNDTRYVGIRYFKVEDTELGKTEIETLYSDEPDPSILKDFWGNYLLYNKRYYLLNLLRTDKSITQ
NSNFLNINQQRGVYQKPNIFSNTRLYTGVEVIIRKNGSTDISNTDNFVRKNDLAYINVVDRDVEYRLYADISIAK
PEKIIKLIRTSNSNNSLGQIIVMDSIGNNCTMNFQNNNGGNIGLLGFHSNNLVASSWYYNNIRKNTSSNGCFWSF
ISKEHGWQEN
The exogenous furin cleavage site (underlined) replaces part of the endogenous BoNT/F
activation loop (bold).
SEQ ID NO: 76 engineered BoNT/G derived from SEQ ID NO: 31
MPVNIKXFNYNDPINNDDIIMMEPENDPGPGTYYKAFRIIDRIWIVPERFTYGFQPDQFNASTGVESK
DVYEYYDPTYLKTDAEKDKFLKTMIKLENRINSKPSGQRLLDMIVDAIPYLGNASTPPDKFAANVANV
SINKKIIQPGAEDQIKGLMTNLIIFGPGPVLSDNFTDSMIMNGHSPISEGFGARMMIRFCPSCLNVEN
NVQENKDTSIFSRRAYFADPALTLMHELIHVLHGLYGIKISNLPITPNTKEFFMQHSDPVQAEELYTE
GGHDPSVISPSTDMNIYNKALQNFQDIANRLNIVSSAQGSGIDISLYKQIYKNKYDFVEDPNGKYSVD
KDKFDKLYKALMFGFTETNLAGEYGIKTRYSYFSEYLPPIKTEKLLDNTIYTQNEGENIASKNLKTEF
NGQNKAVNKEAYEEISLEHLVIYRIAMCKPVKQKSSNSRKKRSTSSEQCIIVNNEDLFFIANKDSES
KDLAKAETIAYNTQNNTIENNESIDQLILDNDLSSGIDLPNENTEPFTNEDDIDIPVYIKQSALKKIF
VDGDSLFEYLHAQTFPSNIENLQLTNSLNDALRNNNKVYTFFSTNLVEKANTVVGASLFVNWVKGVID
DFTSESTQKSTIDKVSDVSIIIPYIGPALNVGNETAKENEKNAFEIGGAAILMEFIPELIVPIVGFFT
LESYVGNKGHIIMTISNALKKRDQKWTDMYGLIVSQWLSTVNTQFYTIKERMYNALNNQSQAIEKIIE
DQYNRYSEEDKMNINIDENDIDFKLNQSINLAINNIDDFINQCSISYLMNRMIPLAVKKLKDEDDNLK
RDLLEYIDTNELYLLDEVNILKSKVNRHLKDSIPFDLSLYTKDTILIQVENNYISNISSNAILSLSYR
GGRLIDSSGYGATMNVGSDVIENDIGNGQFKLNNSENSNITAHQSKFVVYDSMEDNESINFWVRTPKY
NNNDIQTYLQNEYTIISCIKNDSGWKVSIKGNRIIWTLIDVNAKSKSIFFEYSIKDNISDYINKWFSI
TITNDRLGNANIYINGSLKKSEKILNLDRINSSNDIDEKLINCTDTTKFVWIKDENIFGRELNATEVS
SLYWIQSSTNTLKDFWGNPLRYDTQYYLENQGMQNIYIKYFSKASMGETAPRTNENNAAINYQNLYLG
LRFIIKKASNSRNINNDNIVREGDYIYLNIDNISDESYRVYVLVNSKEIQTQLFLAPINDDPTFYDVL
QIKKYYEKTTYNCQILCEKDTKTFGLFGIGKFVKDYGYVWDTYDNYFCISQWYLRRISENINKLRLGC
NWQFIPVDEGWTE
The exogenous furin cleavage site (underlined) replaces part of the endogenous BoNT/G
activation loop (bold).
SEQ ID NO: 77 engineered BoNT/X derived from SEQ ID NO: 32
MKLEINKFNYNDPIDGINVITMRPPRHSDKINKGKGPFKAFQVIKNIWIVPERYNFTNNTNDLNIPSE
PIMEADAIYNPNYLNTPSEKDEFLQGVIKVLERIKSKPEGEKLLELISSSIPLPLVSNGALTLSDNET
IAYQENNNIVSNLQANLVIYGPGPDIANNATYGLYSTPISNGEGTLSEVSFSPFYLKPFDESYGNYRS
LVNIVNKFVKREFAPDPASTLMHELVHVTHNLYGISNRNFYYNEDTGKIETSRQQNSLIFEELLTEGG
IDSKAISSLIIKKIIETAKNNYTTLISERLNTVTVENDLLKYIKNKIPVQGRLGNEKLDTAEFEKKLN
TILFVLNESNLAQRFSILVRKHYLKERPIDPIYVNILDDNSYSTLEGENISSQGSNDFQGQLLESSYF
EKIESNALRAFIKICPRNGLKQKSSNSRKKRSTSCSLLNGCIEVENKDLFLISNKDSLNDINLSEE
KIKPETTVFFKDKLPPQDITLSNYDFTEANSIPSISQQNILERNEELYEPIRNSLFEIKTIYVDKLTT
FHFLEAQNIDESIDSSKIRVELTDSVDEALSNPNKVYSPEKNMSNTINSIETGITSTYIFYQWLRSIV
KDFSDETGKIDVIDKSSDTLAIVPYIGPLLNIGNDIRHGDFVGAIELAGITALLEYVPEFTIPILVGL
EVIGGELAREQVEAIVNNALDKRDQKWAEVYNITKAQWWGTIHLQINTRLAHTYKALSRQANAIKMNM
EFQLANYKGNIDDKAKIKNAISETEILLNKSVEQAMKNTEKFMIKLSNSYLTKEMIPKVQDNLKNEDL
ETKKTLDKFIKEKEDILGTNLSSSLRRKVSIRLNKNIAFDINDIPFSEFDDLINQYKNEIEDYEVLNL
GAEDGKIKDLSGTTSDINIGSDIELADGRENKAIKIKGSENSTIKIAMNKYLRFSATDNESISFWIKH
PKPTNLLNNGIEYTLVENENQRGWKISIQDSKLIWYLRDHNNSIKIVTPDYIAFNGWNLITITNNRSK
GSIVYVNGSKIEEKDISSIWNTEVDDPIIFRLKNNRDTQAFTLLDQFSIYRKELNQNEVVKLYNYYEN
SNYIRDIWGNPLQYNKKYYLQTQDKPGKGLIREYWSSFGYDYVILSDSKTITFPNNIRYGALYNGSKV
LIKNSKKLDGLVRNKDFIQLEIDGYNMGISADRENEDTNYIGTTYGTTHDLTTDFEIIQRQEKYRNYC
QLKTPYNIFHKSGLMSTETSKPTFHDYRDWVYSSAWYFQNYENLNLRKHTKINWYFIPKDEGWDED
The exogenous furin cleavage site (underlined) replaces part of the endogenous BoNT/X
activation loop (bold).
SEQ ID NO: 78 engineered BoNT/X derived from SEQ ID NO:32
MKLEINKFNYNDPIDGINVITMRPPRHSDKINKGKGPFKAFQVIKNIWIVPERYNFTNNTNDLNIPSE
PIMEADAIYNPNYLNTPSEKDEFLQGVIKVLERIKSKPEGEKLLELISSSIPLPLVSNGALTLSDNET
IAYQENNNIVSNLQANLVIYGPGPDIANNATYGLYSTPISNGEGTLSEVSFSPFYLKPFDESYGNYRS
LVNIVNKFVKREFAPDPASTLMHELVHVTHNLYGISNRNFYYNEDTGKIETSRQQNSLIFEELLTEGG
IDSKAISSLIIKKIIETAKNNYTTLISERLNTVTVENDLLKYIKNKIPVQGRLGNEKLDTAEFEKKLN
TILFVLNESNLAQRFSILVRKHYLKERPIDPIYVNILDDNSYSTLEGFNISSQGSNDFQGQLLESSYF
EKIESNALRAFIKICPRNGLLYNAIYRNSKQKSSNSRKKRSTSTNVSYPCSLLNGCIEVENKDLEL
ISNKDSLNDINLSEEKIKPETTVFFKDKLPPQDITLSNYDFTEANSIPSISQQNILERNEELYEPIRN
SLFEIKTIYVDKLTTFHFLEAQNIDESIDSSKIRVELTDSVDEALSNPNKVYSPEKNMSNTINSIETG
ITSTYIFYQWLRSIVKDESDETGKIDVIDKSSDTLAIVPYIGPLLNIGNDIRHGDFVGAIELAGITAL
LEYVPEFTIPILVGLEVIGGELAREQVEAIVNNALDKRDQKWAEVYNITKAQWWGTIHLQINTRLAHT
YKALSRQANAIKMNMEFQLANYKGNIDDKAKIKNAISETEILLNKSVEQAMKNTEKEMIKLSNSYLTK
EMIPKVQDNLKNEDLETKKTLDKFIKEKEDILGTNLSSSLRRKVSIRLNKNIAFDINDIPFSEEDDLI
NQYKNEIEDYEVLNLGAEDGKIKDLSGTTSDINIGSDIELADGRENKAIKIKGSENSTIKIAMNKYLR
FSATDNESISFWIKHPKPTNLLNNGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLLDQFSIYRK
ELNQNEVVKLYNYYFNSNYIRDIWGNPLQYNKKYYLQTQDKPGKGLIREYWSSFGYDYVILSDSKTIT
FPNNIRYGALYNGSKVLIKNSKKLDGLVRNKDFIQLEIDGYNMGISADRENEDTNYIGTTYGTTHDLT
TDFEIIQRQEKYRNYCQLKTPYNIFHKSGLMSTETSKPTFHDYRDWVYSSAWYFQNYENLNLRKHTKT
NWYFIPKDEGWDED
The exogenous furin cleavage site (underlined) replaces part of the endogenous BoNT/X
activation loop (bold).
EXAMPLES

The invention will be further clarified by the following examples, which are intended to be purely exemplary of the invention and are in no way limiting.

### Example 1—Design and Production of BoNT/A1 with the Endogenous Activation Loop Replaced by a Furin Cleavage Site

The BoNT/A1 of SEQ ID NO: 68 was modified to replace a portion of the activation loop (amino acid residues 435-448 of SEQ ID NO: 25) by a furin cleavage site (SEQ ID NO: 5) creating engineered BoNT/A_{1-furin} protein SEQ ID NO: 24 (SXN104539). The DNA construct encoding SEQ ID NO: 24 was created by using pair of oligonucleotides to replace codons in the activation loop in the plasmid encoding SEQ ID NO: 25, with the desired furin sequence by regular substitution mutagenesis. The target protein (SXN104539) was expressed in E. coli BL21 DE3 cells and purified using classical chromatography techniques after lysis. This involved an initial capture step by hydrophobic interaction chromatography followed by anionic-exchange chromatography and buffer exchange into PBS pH 7.2.

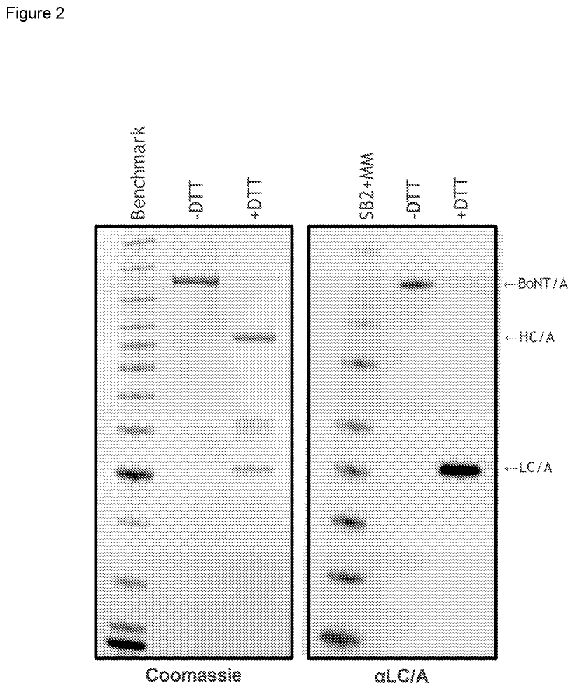

1.2 μg SXN104539 was incubated with 1 μg furin in 24 μL for 48 h at 25° C. and samples in the presence or absence of DTT (reduced or non-reduced, respectively) were resolved by SDS-PAGE (FIG. 2). This gel confirms the presence of a ˜50 kDa and ˜100 kDa chain that was linked by a disulphide bond, with reduced human furin appearing as a band at ˜60 kDa. The ˜50 kDa band was confirmed to be LC/A by Western blot (FIG. 2).

### Example 2—Comparison of the Potency of BoNT/A_{1-furin} with Native BoNT/A₁ and Recombinant BoNT/A₁

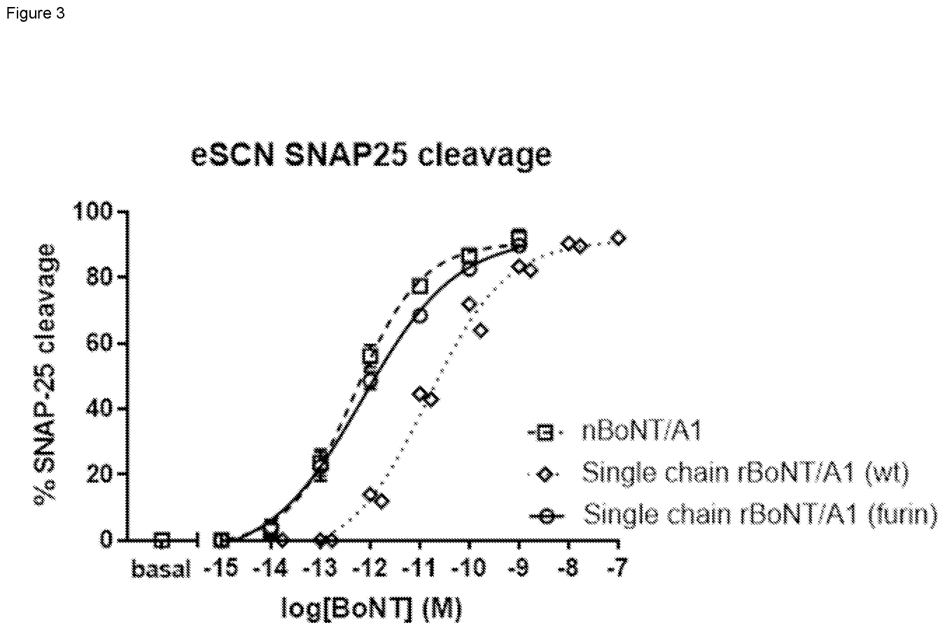

The ability of the engineered BoNT/A_{1-furin} (SXN104539) to enter neurons and cleave SNAP-25 (the target of BoNT/A₁) was assessed using rat embryonic spinal cord neurons (eSCN). FIG. 3 shows that SXN 104539 showed the same pEC₅₀ compared with the native BONT/A₁ (LIST, which is predominantly in the di-chain form), but that the single-chain recombinant BoNT/A₁ (scSXN104445) was 1.3 log units (20×) less potent.

| | | rBoNT/A | rBoNT/A(furin) |
|---|---|---|---|
| | | single-chain | single-chain |
| | nBoNT/A LIST | SXN104445 | SXN104539 |
| pEC₅₀ | 12.3 ± 0.1 | 10.8 ± 0.2 | 12.1 ± 0.1 |
| | n = 8 | n = 2 | n = 3 |

Thus, these results show that SXN104539 retained the same ability to enter the neuron and cleave SNAP-25 as native BoNT/A₁ and had improved potency compared with single-chain BoNT/A₁ (scSXN104445).

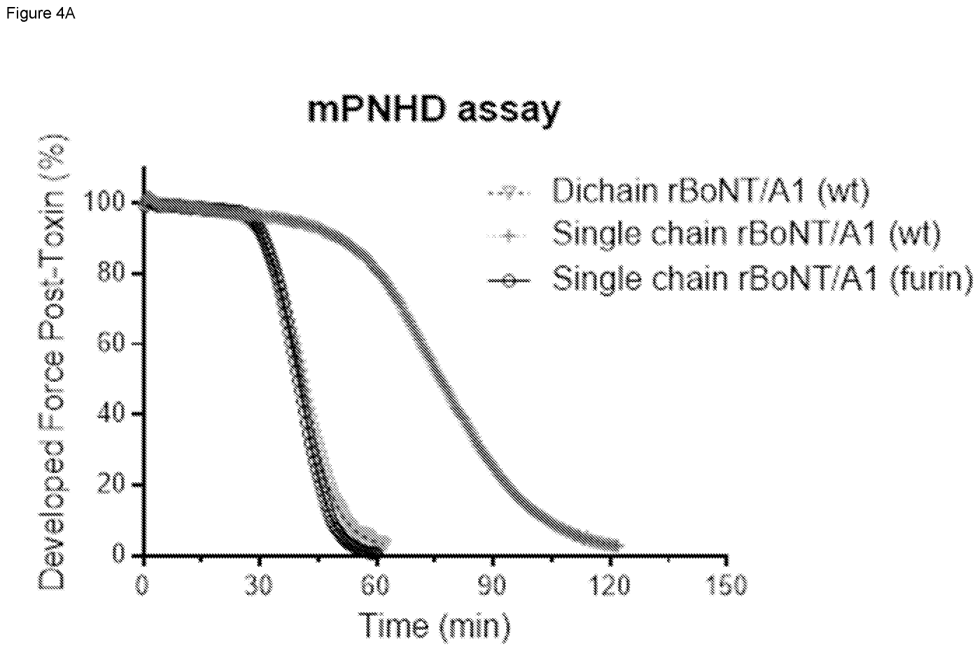

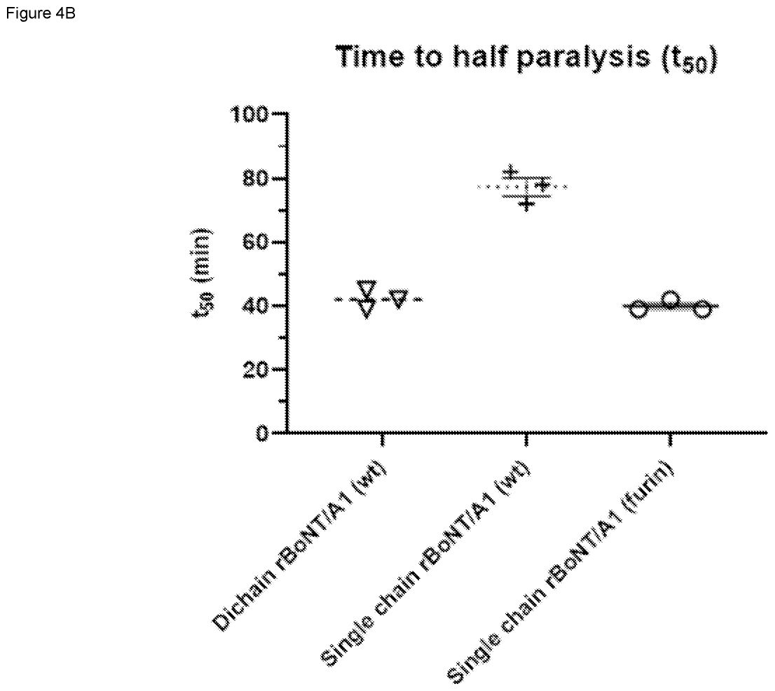

Potency of SXN104539 was further assessed using the mouse phrenic nerve hemi-diaphragm assay (mPNHD). FIG. 4 shows that single-chain rBoNT/A with furin loop (SXN104539) was equipotent to recombinant BoNT/A di-chain (SXN102342) (p>0.05 1w ANOVA).

| | t₅₀ (mean ± sem), n = 3 |
|---|---|
| rBoNT/A scSXN104445 | 77 ± 3 min |
| rBoNT/A(furin) sc SXN104539 | 40 ± 1 min |
| rBoNT/A dcSXN102342 | 42 ± 2 min |

These potency result demonstrate that replacement of a clostridial neurotoxin endogenous activation loop with a furin cleavage site is a successful strategy for in vivo activation of clostridial neurotoxin.

### Example 3—Comparison of Tolerance to BoNT/A_{1-furin}, Native BoNT/A₁, Recombinant Di-Chain BoNT/A₁ and Recombinant Single-Chain BoNT/A₁

The in vivo mouse Digital Abduction Score (DAS) assay was used to assess potency as well as safety relative to native BoNT/A₁ and rBoNT/A₁.

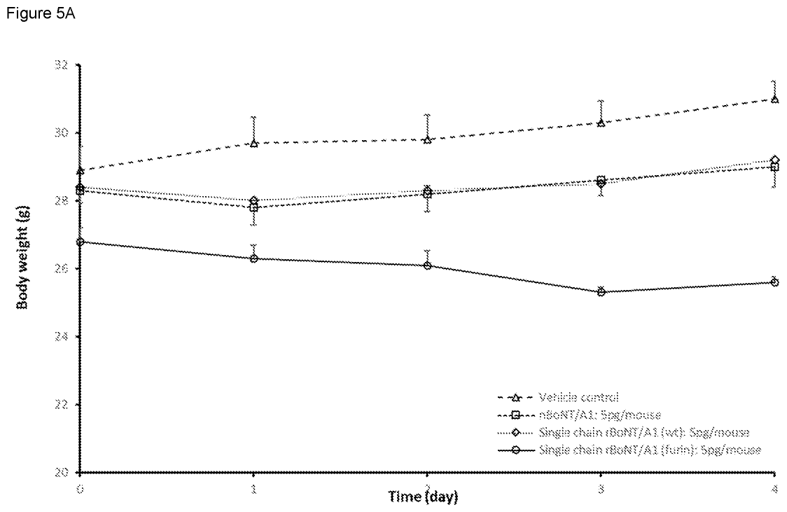

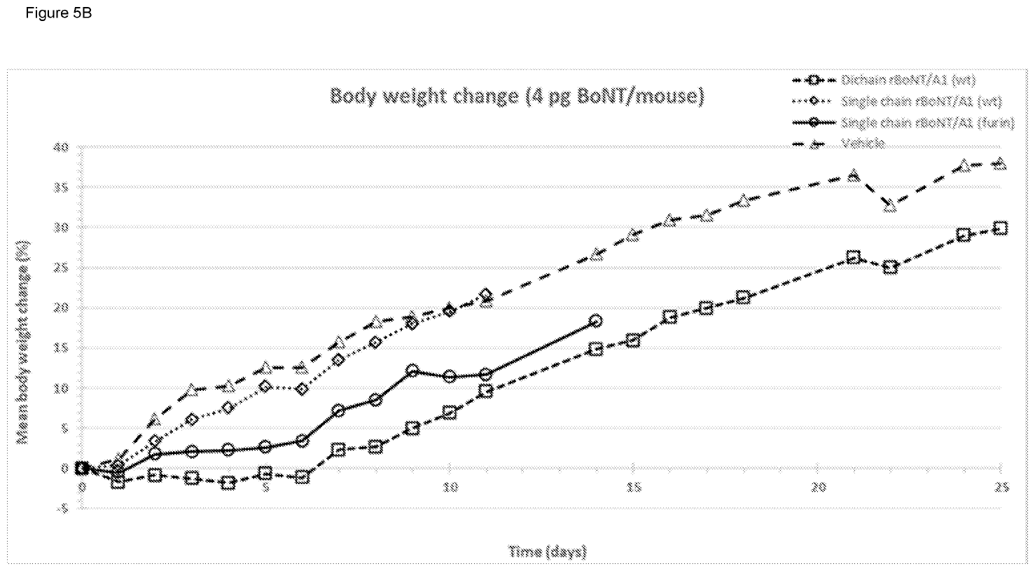

There was an observable decrease in body weight (BW) from 5 pg/mouse i.m. (FIG. 5A). BW loss at 5 pg/mouse i.m. for all molecules tested with maximum BW decrease D1 and recovery on D3 for nBoNT/A1 and scSXN104445 (MTD reached). For SXN104539 maximum BW decrease was observed on D3, but was well tolerated (less than 10% decrease in body weight).

In a second experiment over a longer period, single-chain rBoNT/A with furin loop (SXN104539) elicited a smaller effect on BW compared with recombinant BoNT/A1 di-chain (FIG. 5B), suggesting that SXN104539 is potentially better tolerated than recombinant BONT/A1 di-chain.

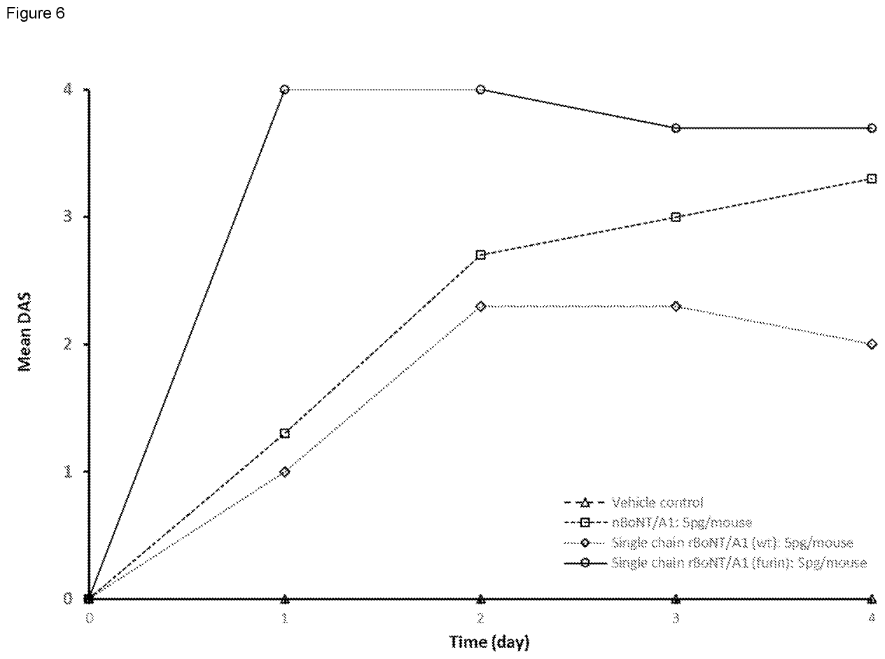

DAS 4 was not reached over a four-day time course t for scSXN104445 or with nBoNT/A1 at 5 pg/mouse (which dose of nBoNT/A1 induced BW loss). The DAS score by order of magnitude were SXN104539>nBoNT/A1>scSXN104445 (FIG. 6).

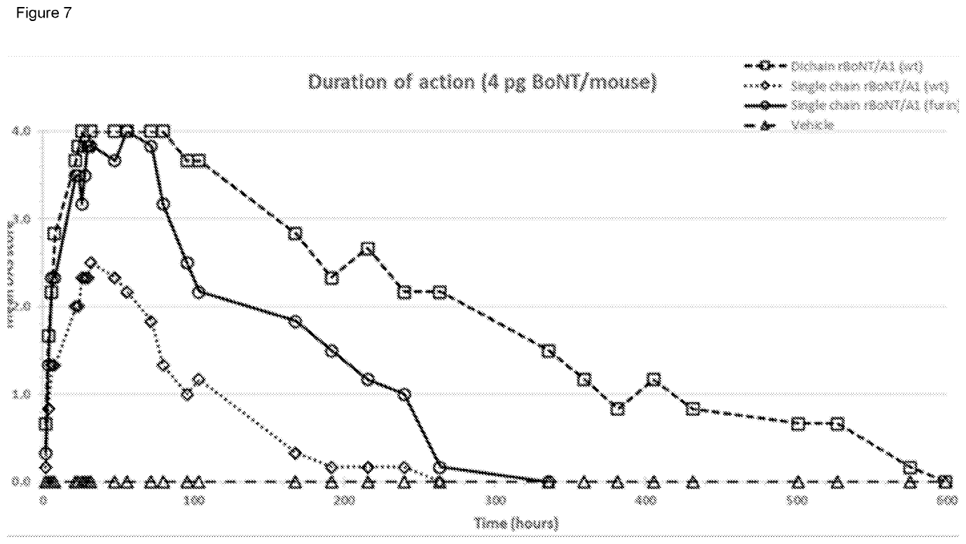

Duration of action was also assessed. A lower dose (4 pg/mouse) was tested and DAS scores determined. DAS 4 was achieved for both dc rBoNT/A1 and SXN104539. FIG. 7 shows that the duration of action for the molecules tests was dc rBoNT/A1>SXN104539>sc rBoNT/A1.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. An engineered clostridial neurotoxin, comprising a furin cleavage site, wherein cleavage at said furin cleavage site results in the production of a di-chain form of the engineered clostridial neurotoxin.

2. The engineered clostridial neurotoxin according to claim 1, wherein the furin cleavage site comprises an amino acid sequence Arg-Xaa-Xaa-Arg (SEQ ID NO: 1), preferably Arg-Xaa-Lys/Arg-Arg (SEQ ID NOs: 2 and 3), even more preferably Arg-Lys-Lys-Arg (SEQ ID No: 4), and even more preferably KQKSSNSRKKR (SEQ ID NO: 5).

3. The engineered clostridial neurotoxin according to claim 1 or 2, wherein the engineered clostridial neurotoxin comprises an exogenous activation loop which comprises or consists of any one of SEQ ID NOs: 14 to 22), preferably SEQ ID NO: 22.

4. The engineered clostridial neurotoxin according to any one of the preceding claims, wherein an endogenous activation loop of a clostridial neurotoxin or part thereof has been replaced by a furin cleavage site.

5. The engineered clostridial neurotoxin according to claim 4, wherein the endogenous neurotoxin activation loop is one or more selected from SEQ ID NO: 34 to 57.

6. The engineered clostridial neurotoxin according to any one of the preceding claims, wherein the clostridial neurotoxin is:
(a) a Botulinum Neurotoxin (BoNT) serotype A, serotype B, serotype C, serotype D, serotype E, serotype F, serotype G or serotype X, or a Tetanus Neurotoxin (TeNT); or
(b) a chimeric BoNT or a hybrid BONT.

7. The engineered clostridial neurotoxin according to claim 6 which is BoNT/A, optionally BONT/A1.

8. The engineered clostridial neurotoxin according to any one of the preceding claims, which is a single-chain clostridial neurotoxin:
(a) encoded by a nucleotide sequence having at least 70% sequence identity to SEQ ID NO: 23; and/or
(b) comprising a polypeptide sequence having at least 70% sequence identity to one or more of SEQ ID NOs: 24 or 70 to 78.

9. The engineered clostridial neurotoxin according to any one of the preceding claims, which is a re-targeted clostridial neurotoxin in which an endogenous H_{C} or H_{CC} of a clostridial neurotoxin is replaced by an exogenous targeting moiety (TM).

10. An engineered BoNT/A comprising a furin cleavage site, which comprises a polypeptide sequence having at least 70% sequence identity, preferably at least 80%, more preferably at least 90%, even more preferably at least 95% sequence identity to SEQ ID NO: 24.

11. A method for proteolytically processing an engineered clostridial neurotoxin according to any one of claims 1 to 9 or an engineered BoNT/A according to claim 10 into a corresponding di-chain clostridial neurotoxin or BoNT/A, the method comprising contacting the engineered clostridial neurotoxin or engineered BoNT/A with furin, thereby producing a di-chain clostridial neurotoxin or BoNT/A.

12. A di-chain clostridial neurotoxin or BoNT/A obtainable by the method of claim 11.

13. A polynucleotide encoding an engineered clostridial neurotoxin as defined in any one of claims 1 to 9 or an engineered BoNT/A according to claim 10.

14. An expression vector comprising a polynucleotide as defined in claim 13, which is operably linked to a promoter.

15. A polynucleotide according to claim 13, or an expression vector according to claim 14, wherein said polynucleotide or expression vector:
(a) comprises a nucleotide sequence having at least 70% sequence identity to SEQ ID NO: 23; and/or
(b) encodes a polypeptide sequence having at least 70% sequence identity to one or more of SEQ ID NOs: 24 or 70 to 78.

16. A method of producing an engineered clostridial neurotoxin as defined in any one of claims 1 to 9, or an engineered BoNT/A according to claim 10 comprising the step of expressing a polynucleotide as defined in claim 13 or 15 or an expression vector as defined in claim 14 or 15 in a cell, and recovering the expressed engineered clostridial neurotoxin or engineered BoNT/A.

17. The method of claim 16, which further comprises a step of introducing a polynucleotide as defined in claim 13 or 15 or an expression vector as defined in claim 14 or 15 into the cell.

18. A cell expressing an engineered clostridial neurotoxin as defined in any one of claims 1 to 9 or an engineered BoNT/A according to claim 10.

19. The cell of claim 18, which comprises a polynucleotide as defined in claim 13 or 15, or an expression vector as defined in claim 14 or 15.

20. A pharmaceutical composition comprising an engineered clostridial neurotoxin as defined in any one of claims 1 to 9, an engineered BoNT/A according to claim 10, or a di-chain clostridial neurotoxin or di-chain BoNT/A as defined in claim 12, and a pharmaceutically acceptable carrier, excipient, diluent, adjuvant, propellant and/or salt.

21. An engineered clostridial neurotoxin as defined in any one of claims 1 to 9, an engineered BoNT/A according to claim 10, a di-chain clostridial neurotoxin or di-chain BONT/A as defined in claim 12, or a pharmaceutical composition as defined in claim 20, for use in a method of preventing or treating a disease or disorder for which a therapy with a botulinum neurotoxin is indicated, wherein optionally said disease or disorder is selected from a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder.

22. Use of an engineered clostridial neurotoxin as defined in any one of claims 1 to 9, an engineered BoNT/A according to claim 10, a di-chain clostridial neurotoxin or di-chain BONT/A as defined in claim 12, or a pharmaceutical composition as defined in claim 20, in the manufacture of a medicament for preventing or treating a disease or disorder for which a therapy with a botulinum neurotoxin is indicated, wherein optionally said disease or disorder is selected from a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder.

23. A cosmetic composition comprising an engineered clostridial neurotoxin as defined in any one of claims 1 to 9, an engineered BoNT/A according to claim 10, or a di-chain clostridial neurotoxin or di-chain BoNT/A as defined in claim 12, and a cosmetically acceptable carrier, excipient, diluent, adjuvant, propellant and/or salt.

24. Use of a cosmetic composition as defined in claim 23, for preventing or alleviating a cosmetic indication for which the application of a botulinum neurotoxin is indicated.

25. A method for proteolytically processing a single-chain clostridial neurotoxin into a corresponding di-chain clostridial neurotoxin, the method comprising:
(a) providing a single-chain clostridial neurotoxin; and
(b) contacting the single-chain clostridial neurotoxin with furin;
wherein the single-chain clostridial neurotoxin has an activation loop comprising or consisting of the polypeptide sequence Arg-Xaa-Xaa-Arg (SEQ ID NO: 1); and
wherein furin hydrolyses a peptide bond of the activation loop thereby producing a di-chain clostridial neurotoxin.

26. The method according to claim 25, wherein the activation loop comprises or consists of:
(a)
(SEQ ID NOs: 2 or 3)
Arg-Xaa-Lys/Arg-Arg;
(b)
(SEQ ID No: 4)
Arg-Lys-Lys-Arg;
and/or
(c)
(SEQ ID NO: 5)
KQKSSNSRKKR

27. The method according to claim 25 or 26, wherein the single-chain clostridial neurotoxin:
(a) is an engineered clostridial neurotoxin as defined in any one of claims 1 to 9;
(b) is encoded by a nucleotide sequence having at least 70% sequence identity to SEQ ID NO: 23;
(c) comprises a polypeptide sequence having at least 70% sequence identity to one or more of SEQ ID NOs: 24 or 70 to 78.

28. A clostridial neurotoxin, or a pharmaceutical composition comprising said clostridial neurotoxin, for use in a method of preventing or treating a disease or disorder for which a therapy with a botulinum neurotoxin is indicated, wherein the clostridial neurotoxin is administered to a subject in single-chain form.

29. The clostridial neurotoxin, or a pharmaceutical composition for use according to claim 28, wherein the clostridial neurotoxin or pharmaceutical composition is substantially free of a di-chain form of the clostridial neurotoxin.

30. The clostridial neurotoxin, or a pharmaceutical composition for use according to claim 29, wherein the clostridial neurotoxin or pharmaceutical composition comprises less than 400 pg di-chain clostridial neurotoxin per 100 ng single-chain clostridial neurotoxin, or less than 300 pg di-chain clostridial neurotoxin per 100 ng single-chain clostridial neurotoxin, or less than 200 pg di-chain clostridial neurotoxin per 100 ng single-chain clostridial neurotoxin, or less than 100 pg di-chain clostridial neurotoxin per 100 ng single-chain clostridial neurotoxin, or less than 50 pg di-chain clostridial neurotoxin per 100 ng single-chain clostridial neurotoxin.

31. The clostridial neurotoxin, or a pharmaceutical composition for use according to any one of claims 28 to 30, wherein said disease or disorder is selected from a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder.

32. Use of a cosmetic composition comprising a single-chain clostridial neurotoxin, and a cosmetically acceptable carrier, excipient, diluent, adjuvant, propellant and/or salt for preventing or alleviating a cosmetic indication for which the application of a botulinum neurotoxin is indicated, wherein the single-chain clostridial neurotoxin is administered to a subject in single-chain form.


SEQUENCE LISTING (78 sequences)

SEQ ID NO: 1 - protein, 4 aa, Artificial Sequence
  furin cleavage consensus sequence
  misc_feature (2)..(3): Xaa can be any naturally occurring amino acid
RXXR

SEQ ID NO: 2 - protein, 4 aa, Artificial Sequence
  furn cleavage site consensus sequence
  misc_feature (2)..(2): Xaa can be any naturally occurring amino acid
RXKR

SEQ ID NO: 3 - protein, 4 aa, Artificial Sequence
  furn cleavage site consensus sequence
  misc_feature (2)..(2): Xaa can be any naturally occurring amino acid
RXRR

SEQ ID NO: 4 - protein, 4 aa, Artificial Sequence
  furin cleavage site
RKKR

SEQ ID NO: 5 - protein, 11 aa, Artificial Sequence
  furin cleavage site
KQKSSNSRKKR

SEQ ID NO: 6 - protein, 6 aa, Artificial Sequence
  furin cleavage site
SRKKRS

SEQ ID NO: 7 - protein, 6 aa, Artificial Sequence
  furin cleavage site
SRKRRS

SEQ ID NO: 8 - protein, 7 aa, Artificial Sequence
  furin cleavage site
SKRKKRS

SEQ ID NO: 9 - protein, 7 aa, Artificial Sequence
  furin cleavage site
SKRKRRS

SEQ ID NO: 10 - protein, 14 aa, Artificial Sequence
  furin cleavage site
TSSKSRRRKKRSTS

SEQ ID NO: 11 - protein, 14 aa, Artificial Sequence
  furin cleavage site
AGLITGGRRTRRSI

SEQ ID NO: 12 - protein, 13 aa, Artificial Sequence
  furin cleavage site
KVADSLSTRKQKR

SEQ ID NO: 13 - protein, 20 aa, Artificial Sequence
  furin cleavage site
LATGLRNTSQRSRRRKKRGL

SEQ ID NO: 14 - protein, 25 aa, Artificial Sequence
  exogenous activation loop based on BoNT/A1 activation loop with furin cleavage site
CVRGIITSKTKSLSRKKRSALNDLC

SEQ ID NO: 15 - protein, 25 aa, Artificial Sequence
  exogenous activation loop based on BoNT/A1 activation loop with furin cleavage site
CVRGIITSKTKSLSRKRRSALNDLC

SEQ ID NO: 16 - protein, 25 aa, Artificial Sequence
  exogenous activation loop based on BoNT/A1 activation loop with furin cleavage site
CVRGIITSKTKSSKRKKRSALNDLC

SEQ ID NO: 17 - protein, 25 aa, Artificial Sequence
  exogenous activation loop based on BoNT/A1 activation loop with furin cleavage site
CVRGIITSKTKSSKRKRRSALNDLC

SEQ ID NO: 18 - protein, 25 aa, Artificial Sequence
  exogenous activation loop based on BoNT/A1 activation loop with furin cleavage site
CVRGITSSKSRRRKKRSTSALNDLC

SEQ ID NO: 19 - protein, 25 aa, Artificial Sequence
  exogenous activation loop based on BoNT/A1 activation loop with furin cleavage site
CVRGIAGLITGGRRTRRSIALNDLC

SEQ ID NO: 20 - protein, 25 aa, Artificial Sequence
  exogenous activation loop based on BoNT/A1 activation loop with furin cleavage site
CVRGIIKVADSLSTRKQKRALNDLC

SEQ ID NO: 21 - protein, 31 aa, Artificial Sequence
  exogenous activation loop based on BoNT/A1 activation loop with furin cleavage site
CVRGILATGLRNTSQRSRRRKKRGLALNDLC

SEQ ID NO: 22 - protein, 25 aa, Artificial Sequence
  exogenous activation loop based on BoNT/A1 activation loop with furin cleavage site
CVRGIKQKSSNSRKKRSTSALNDLC

SEQ ID NO: 23 - DNA, 3888 bp, Artificial Sequence
  nucleic acid sequence of BoNT/A1 with a furin cleavage site
atgccgtttgtgaacaaacagttcaactataaagatccggtgaacggtgttgatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattaaacaaaaatcttcgaactcaagaaaaaagcgaagtacaagtgccctgaatgacctgtgcattaaagtgaataattgggacctgttttttagcccgagcgaagataactttaccaacgatctgaataaaggcgaagaaattaccagcgataccaatattgaagcagccgaagaaaacattagcctggatctgattcagcagtattatctgaccttcaactttgataacgagccggaaaatatcagcattgaaaatctgagcagcgatattattggtcagctggaactgatgccgaatattgaacgttttccgaacggcaaaaaatacgagctggataaatacaccatgttccattatctgcgtgcccaagaatttgaacatggtaaaagccgtattgccctgaccaattcagttaatgaagcactgctgaacccgagccgtgtttataccttttttagcagcgattacgtgaaaaaggtgaacaaagcaaccgaagcagcaatgtttttaggttgggttgaacagctggtgtatgatttcaccgatgaaaccagcgaagttagcaccaccgataaaattgcagatatcaccattatcatcccgtatattggtccggcactgaatattggcaatatgctgtataaagatgatttcgtgggtgccctgatttttagcggtgcagttattctgctggaatttattccggaaattgccattccggttctgggcacctttgcactggttagctatattgcaaataaagttctgaccgtgcagaccattgataatgcactgagcaaacgtaacgagaaatgggatgaagtgtacaaatatatcgtgaccaattggctggccaaagttaatacccagattgatctgatccgcaaaaaaatgaaagaagccctggaaaatcaggcagaagcaaccaaagccattatcaactatcagtataaccagtacaccgaagaagagaaaaacaacatcaacttcaacatcgatgacctgagcagcaaactgaatgaaagcatcaataaggccatgattaacatcaacaaatttctgaatcagtgcagcgtgagctatctgatgaatagcatgattccgtatggtgtgaaacgcctggaagattttgatgcaagcctgaaagatgcgctgctgaaatatatctatgataatcgtggcaccctgattggccaggttgatcgtctgaaagataaagttaacaataccctgagtaccgacattccgtttcagctgagcaaatatgttgataatcagcgtctgctgagcacctttaccgaatatatcaagaacatcatcaacaccagcattctgaatctgcgctatgaaagcaatcatctgatcgatctgagccgttatgcaagcaaaatcaacattggtagcaaagtgaacttcgacccgattgataaaaaccagattcagctgtttaatctggaaagcagcaaaatcgaagtgatcctgaaaaacgccattgtgtataacagcatgtatgagaatttctcgaccagcttttggattcgcattccgaaatactttaatagcatcagcctgaacaacgagtacaccattattaactgcatggaaaacaatagcggttggaaagtgagcctgaattatggtgaaattatctggaccctgcaggatacccaagaaatcaaacagcgtgttgtgttcaaatacagccagatgattaatatcagcgactatatcaaccgctggatctttgttaccattaccaataatcgcctgaataacagcaagatctatattaacggtcgcctgattgatcagaaaccgattagcaatctgggcaatattcatgcgagcaacaacattatgtttaaactggatggttgccgtgatacccatcgttatatttggatcaaatacttcaacctgtttgataaagaactgaacgaaaaagaaattaaagacctgtacgacaaccagagcaattccggtattctgaaagacttttggggagattatctgcagtatgacaaaccgtattatatgctgaacctgtatgacccgaacaaatatgtggatgtgaacaatgttggtatccgtggctatatgtatctgaaaggtccgcgtggtagcgttatgaccaccaacatttatctgaatagcagcctgtatcgcggtacgaaatttatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagcgcactggaaattccggatgttggtaatctgagccaggttgttgttatgaaaagcaaaaatgatcagggcatcaccaacaagtgcaaaatgaatctgcaggacaataacggcaacgatattggttttattggcttccaccagttcaacaatattgcgaaactggttgcaagcaattggtataatcgtcagattgaacgtagcagtcgtaccctgggttgtagctgggaatttatccctgtggatgatggttggggtgaacgtccgctg

SEQ ID NO: 24 - protein, 1296 aa, Artificial Sequence
  amino acid sequence of BoNT/A1 with a furin cleavage site
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIKQKSSNSRKKRSTSALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 25 - protein, 1296 aa, Clostridium botulinum [sequence omitted]

SEQ ID NO: 26 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 27 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 28 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 29 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 30 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 31 - protein, 1297 aa, Clostridium botulinum
  misc_feature (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 32 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 33 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 34 - protein, 14 aa, Clostridium botulinum
CLRLTKNSRDDSTC

SEQ ID NO: 35 - protein, 14 aa, Clostridium botulinum
CLRLTRNSRDDSTC

SEQ ID NO: 36 - protein, 17 aa, Clostridium botulinum
CHKAIDGRSLYNKTLDC

SEQ ID NO: 37 - protein, 25 aa, Clostridium botulinum
CVRGIITSKTKSLDEGYNKALNELC

SEQ ID NO: 38 - protein, 25 aa, Clostridium botulinum
CVRGIITSKTKSLDEGYNKALNDLC

SEQ ID NO: 39 - protein, 25 aa, Clostridium botulinum
CVRGIITSKTKSLDKGYNKALNDLC

SEQ ID NO: 40 - protein, 25 aa, Clostridium botulinum
CVRGIIPFKTKSLDEGYNKALNYLC

SEQ ID NO: 41 - protein, 25 aa, Clostridium botulinum
CVRGIIPFKTKSLDEGYNKALNDLC

SEQ ID NO: 42 - protein, 11 aa, Clostridium botulinum
CSNSNTKNSLC

SEQ ID NO: 43 - protein, 15 aa, Clostridium botulinum
CKNIVSVKGIRKSIC

SEQ ID NO: 44 - protein, 15 aa, Clostridium botulinum
CTNIFSPKGIRKSIC

SEQ ID NO: 45 - protein, 15 aa, Clostridium botulinum
CKNIVFSKGITKSIC

SEQ ID NO: 46 - protein, 15 aa, Clostridium botulinum
CKNIVFSKGIRKSIC

SEQ ID NO: 47 - protein, 15 aa, Clostridium botulinum
CKSIVSKKGTKNSLC

SEQ ID NO: 48 - protein, 15 aa, Clostridium botulinum
CLNSSFKKNTKKPLC

SEQ ID NO: 49 - protein, 17 aa, Clostridium botulinum
CKSVIPRKGTKAPPRLC

SEQ ID NO: 50 - protein, 17 aa, Clostridium botulinum
CKSIIPRKGTKAPPRLC

SEQ ID NO: 51 - protein, 17 aa, Clostridium botulinum
CKSIIPRKGTKQSPSLC

SEQ ID NO: 52 - protein, 29 aa, Clostridium tetani
CKKIIPPTNIRENLYNRTASLTDLGGELC

SEQ ID NO: 53 - protein, 15 aa, Clostridium botulinum
CKPVMYKNTGKSEQC

SEQ ID NO: 54 - protein, 10 aa, Clostridium botulinum
CKSVKVPGIC

SEQ ID NO: 55 - protein, 10 aa, Clostridium botulinum
CKSVRAPGIC

SEQ ID NO: 56 - protein, 10 aa, Clostridium botulinum
CKSVKAPGIC

SEQ ID NO: 57 - protein, 45 aa, Clostridium botulinum
CPRNGLLYNAIYRNSKNYLNNIDLEDKKTTSKTNVSYPCSLLNGC

SEQ ID NO: 58 - protein, 5 aa, Clostridium botulinum
  misc_feature (3)..(4): Xaa can be any naturally occurring amino acid
HEXXH

SEQ ID NO: 59 - protein, 794 aa, Homo sapiens
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 60 - protein, 7 aa, Artificial Sequence
  additional protease cleavage site
  misc_feature (1)..(1): Xaa can be any naturally occurring amino acid
  misc_feature (3)..(5): Xaa can be any naturally occurring amino acid
XDXXXLL

SEQ ID NO: 61 - protein, 7 aa, Artificial Sequence
  additional protease cleavage site
  misc_feature (1)..(1): Xaa can be any naturally occurring amino acid
  misc_feature (3)..(5): Xaa can be any naturally occurring amino acid
XEXXXLL

SEQ ID NO: 62 - protein, 7 aa, Artificial Sequence
  additional protease cleavage site
  misc_feature (1)..(1): Xaa can be any naturally occurring amino acid
  misc_feature (3)..(5): Xaa can be any naturally occurring amino acid
XEXXXIL

SEQ ID NO: 63 - protein, 7 aa, Artificial Sequence
  additional protease cleavage site
  misc_feature (1)..(1): Xaa can be any naturally occurring amino acid
  misc_feature (3)..(5): Xaa can be any naturally occurring amino acid
XEXXXLM

SEQ ID NO: 64 - protein, 4 aa, Artificial Sequence
  additional protease cleavage site
  misc_feature (2)..(3): Xaa can be any naturally occurring amino acid
  SITE (4)..(4): Xaa can be any hydrophobic amino acid
YXXX

SEQ ID NO: 65 - protein, 7 aa, Artificial Sequence
  TEV cleavage site
ENLYFQG

SEQ ID NO: 66 - protein, 6 aa, Artificial Sequence
  Thrombin cleavage site
LVPRGS

SEQ ID NO: 67 - protein, 8 aa, Artificial Sequence
  PreScission cleavage site
LEVLFQGP

SEQ ID NO: 68 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 69 - protein, 1304 aa, Artificial Sequence
  non-engineered BoNT/AB chimera
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 70 - protein, 1304 aa, Artificial Sequence
  engineered BoNT/AB chimera
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIKQKSSNSRKKRSTSALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 71 - protein, 1302 aa, Artificial Sequence
  engineered BoNT/B
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKQKSSNSRKKRSTSGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 72 - protein, 1297 aa, Artificial Sequence
  engineered BoNT/C
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIKQKSSNSRKKRSTSTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 73 - protein, 1284 aa, Artificial Sequence
  engineered BoNT/D
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLKQKSSNSRKKRSTSDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 74 - protein, 1257 aa, Artificial Sequence
  engineered BoNT/E
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIKQKSSNSRKKRSTSSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 75 - protein, 1283 aa, Artificial Sequence
  engineered BoNT/F
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIKQKSSNSRKKRSTSRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 76 - protein, 1304 aa, Artificial Sequence
  engineered BoNT/G
  misc_feature (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVKQKSSNSRKKRSTSSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 77 - protein, 1288 aa, Artificial Sequence
  engineered BoNT/X
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLKQKSSNSRKKRSTSCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 78 - protein, 1303 aa, Artificial Sequence
  engineered BoNT/X
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKQKSSNSRKKRSTSTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED